SEQUENCE LISTING (90 sequences)

SEQ ID NO: 1 - DNA, 35056 bp, Homo sapiens
  source 1..35056: mol_type = genomic DNA
gcgcaagaatgtaatagagcttcgacggccgccattttctttctttcttagctgttagctgagaggaagtctctgaacaggcggcagcggctcttatagtgcaaccatggcagactattcaacagtgcctcccccctcttctggctcagctggtggcggtggtggcggcggtggtggtggaggagttaacgacgctttcaaagatgcactgcagagagcccggcaggtaagtgtggaccgcgcggcggaatcccgaaagctcacggtaattggccgctgactgagtaggccgctacccttaagcgcatgaggaagaggaaagaggtgttcttccgggctgaaatgtgaagagacacgtttccccatgttggtaataacgattagagaccagaacccagttttgtgttcttggtgcctaatccacttagaaccccgacgcgtgctacgcaaagaaggcctgaagtctttctcccgcttctgcggcactcgcgtgtctccagtgagctagtttagataaagatcctcttccaggggataaagcgcagttagtttcacacaatttaatggaaggttctggtaatgagtttgggaaagaactagggtctgtcctggagccatagcaagggaagggatttatcattaaagtagcctttacagctcatttccgttctctctcgcaattaaaaccgctttcagtaccattcaccgtcacacctctacaaggaagggacttgaaagcagcctttttctgggcgggatttacgtgtcagtctgttccaccagtccgccccccttatttctcaaaatggcctcaggcccattataccagaggtttcaatttgaatctgcctctcagttcagagtcgtaaactgaccagacctctttgtattacgtagtgcgtgcatttgccctgaaggcaccactttcccagacgaaagctgttaaaatagtgcgtgtattccaggaaaataaaagataccttaatttgaactttacattcttagatagtcccctaatatatttaacatttcaaaatgtatggtgttggtatagatttgcatgtaagcaaaagaatcctattctctgtgacactatgcatattgtactaggtgctgggcattttttactagttttaagctaatgataattagaaaccagtgttgtgctgtgttttcgttgcattaggagttcacttagttaactttttaccgggacagttgaaggaacattgagtcaaaattagaattcataaaatccgttgtaacacatctaatgtgaacgcattataaacatgtacctgtacttttttataaccagaaatactaggagtagtcaacaaaaggtcatcattaatattagttctgcggttttttccacgtaatttaagaaattctgaacatgtttagcaacaagcatataactatgacaaacactcttagcgtgttttattagattgatttgtaaaacttaagggaactattttatttactggaaccaactattttattttaccagaaccagcacattggattaattggtatcgcacactgtaggtagatactggagttttgttttgttttgtttgagacggagtctccctctgtcgcccagactggagtgcagtggtgcgatctcgtcccactgcaacctctgcctcccggcttcaagcaattctcctgcctcagcctcccgagtagctgggattacaggcgcccgccaccatgcccggctaattttttgtgttttttttgtttgtttgtttgttttcagtagagacagggtttcaccatgctggccaggctggtctcgaactcctgacctcgtgatccgcccgtcttggcctcccaaagctgggattacaggcgtgagccgctgcgcccggccttgttttttttcgtttgtttgttttaatgcatgaattgtttcctactaagaagctatgatatagttccttgaccaaatgcagatgaacaggattatctgattaatactttaacgagagcagacaaaatatggatatttaattcatccacttgctttataagtgtttatagagtttgttaggaggggatgcccaatttcctaagtaaaggtgatatatgagcaagacattataaaatgaaagagagtttggccagtgaagaaaagaagcagcatgcgcagaggtgtggggacttgagggaatggcaaggacccagaagtgaagtcgctagaagatgggggtgggaggacgtgaaacgaggcaaggaaaggtacaagcggatccagatactggaagaccttgtgttgcaatgctttaattaaaaattggatttcaggccgggtacggtggctcacgcctgtagtcccagcactttgggaggctgaggcaggcagatcacttgaagtcggagtttaagaccagcctggccaacatggtgaaaccccatctttacaaaaaatacaaaaagtagccgggcttggtggcctgtgcctgtagtcctagctacttgggaggctgaggcacaagaaccgcttcaacctgggaagtggaggttgcagtgagtggagatcacgctgctgcactccagcctggtcatagagcgagaccctgtctcaaaaaaaaaaaaattggattttattattaggctaagccataatttctttatacttcttaaaatatagaaattgttggcaaattgtgaaatttattgttgtataatgctaatagatcagttgtcctgatgtctttgctgtaatgatttctttataaaaatgatcttaaatctgagctccctaactttagtttttgcctggaattacccattacatttgatgatatctctaaatgtcagttgtagctgttactctgatgatataagtggaatatacagaagcgtacttagacaaaagttaggttaatatctgaactacttcctccttgtgtatttaagagaatattgacttaagtttctagaatcctcaactaatcctaagtttattttctttgtctagaatactatgctgtttttgtttttggaaggaagagatataggcatagtttcctgctctcaaggagcttcaaaggctgtaccagtggggatgccattggtatttttagctggatagttgttattcagaaaagcaggacaagtaattatgattcctggtccgtacctggtaatgccagtaatgttaactctagctggttgttgacatctggtcatttagttgccaatcttctttttttttttctggcttttatgtgaaatttttagatttttataatatcctgagctaaattcaacacagggacaccagattgctgctttagttcagggtttccagcctgtgcacttaagaaatttatttttgtgtatatcaagctgtaactccagagattgggattgtttgattgggtctttagcagtggtactaatagcaacttctgtctctagaacattggaaaattaaaatgtgtttatctaccgtttttttcctcgaggttatatgaaggtagaaatgaatcagactagatgattagctaagcgagactattaaccctcatcccttcccctctagacaactatgaaattagtcattatgtattcgatccttcttgcagtctcttctctgacagttataaaagtgatttaggctgcataatgttgtttgaatgaaatgaaaatatagactagagctgtttttttttttatttccatcagtctcttcagtgaaaactaacatttgagcatgattctttttttaaatcattttgtgacagtttagcaaggcttgtgataagcaagttatggtatggtaatatttctagtgtccacgtttcttcacatgtctggtgtatgggaactactaactccatcaggaccttgcctatagtaggtactcaacatttactgaattaaatcaataaacatttttaatgaattacagtacaagtcagacctctgtatctgtgggctctgcatctgcaaattcagccaaccatggatcagaaatattagaaaaatggaagaacagtccagcaatacaagtaatatgaataaaaacaatacaacaactatgtacattgtatcaggtattataagtaatttagagatgctttaagtatactgaaggatttgcgtaggttatatgcagatactgtaccattttatataaggaacttgagcatctgtggattttggtatttgcatggttcctggaaccaatcccccagggatactgagggactatagttgatcataccacctgattttagagattttctgagtctcagaagttaattaagtaaactacaatagtctgttcttaacctcggaggatacattccaagaacctcagtgaatatctgaaaccacagatagtattgaatccaatatatacacggtaatattttttcctatacatatgtatctataaagtttaaattctaaatcagacacagtattaacgataataataaattagtgcaagactgggcatagtggttcacacctataatcttaacactttaactatgacgttgtctttgaaaagaaatcagctagccaaggtggctcatggctgtaatcctagtgttttgagaagctaagtcaggaagattgcttgagcccaggagtctgagaccaccctaggcaacatggtgaaaccctgtttctataaaaaataccaaaaaataggctgggcgcggtggctcacgcctgtaatctcagcactttgggaggctgaggcgggtggatcacgaggtcaggagattgagaccgtcctggctaacacggtgaaaccccgtctctactaaaaatacaaaaaattagccgggagtagtgggcgcctgtagtcccagctactcaggagactgaggcaggagaatggcgtgaacccgggaggtggagcttgcagtgaaccgagatcgcgccactgcatgccagcctgggcaacagagcgagactccatctcaaaaaaaaaaaaaaaaaaaccaaaaaattagccagacgtggtggtgcttgcctgtagtaccagctatccagaaggctgaggtgggaggattgcttgaacttgggaggtcaagtctagaatgttgacaatgttgggtcctttatgtagttgcataagtgagccatgatcgtgccactgcactacatccttgggcaacagcctgaccctgtctcaaaattttaatttaattaaaaaaataaaatagaacaattacaacaatacactgtattactggacaagaagggcaaatttaaaaaaaattaaaccaatatgctataataagttatatgaatgaggggaccctcccctaccccagaatatctgattgtactatatcataggtaactgaaaccgtgaagagcaaaaactgaagataaagagactactgtgtcttttaagtttcttttcaactcccaaattcttggatttctcacctcttggcttcctcaatagaggtgagaaatgttaaagtagtgaaaacaggaaaaataacttactcattcaagaagtagataatggtccagatggaaagcttgaattatttttgtaaaactaaaattaaataaagtagccaggcatggtggcttacgcctgtaatcccagcactttgggaggctgaggcgggtggatcacttgcggtcaggagttcaagaccagcctggccaacaaggtgaaaccctgtctatactaaaaatacaaaaattagctgagcatggtggcgggcgcctgtaatcccagctactcgagaggctgaggcaggagaatcgcttgaacttggggggcggacattgcagtgagcccagatcacgccactgcactctagtctgggtaacatcttgagactccatctcaaataataataataattaaataaagtaaaaagtttcccacacctcataaatgtctaataaaaattgaatatgttgagttcaagtactctgaaaaaggagttgaatatagttggaggttggtttttaggaattactatttttcttaaattaactatccttgtagtcacctaggaattgtgtattttctatagatcttagaaaattatcaaatctacagttcattttgttttttcagttttttttttttttttaagagatggagtcttgctgtattagcgttgaactcctggcctcagccagttctcccatctcagcttctgaagtagctggggctgcaggtgccactgagcctggcttctttattggtatttttattaaacacttttctctaatgtctttgtaacagttctcagtttttgaaatgctgttactgtttctttagtgtgaactgtcaactttcattttttcttttcttttcttttcttttctttttttcttttttgagacagagtctcgctctgtcacccagactggagtgcagtggtgcgatcttggctcactgcgacctctgcctcccgggttcaagtgattctcctgcctgagcctcccgagtagctggaattacaggtgcgcaccactgtgcctggctaattttttttttttttttttgtatttttagtagaggtggtgtttcaccatgtcagtcaggctggtcttgaactcctgaactcatgatcccccccgccctgcctcggcctcccaaagtgctgggattacaggcatgagccaccacgcctggcctcagctttcattttcatttggttagtttttgaactattcagtgggtaaagttgtataaataagtgtcttttctctgtatagaagtgtcttggagttcaaggagtgctgctttgcaaactcatagagtatttataaaagctaactgcagaaggtattcataggctaaaccgtttcctattcttggtagcaccattttctctggcctgaaatactttccttctactattagtgcctgtcgatacccagcagtgtatttactttcctgaggaacaattcaaatgctaagtgctttaagacctaagggtggaaaagcagtgttttcaggcattattaggaaaataagatttaaattagacacccagaaacaaaaacaggtttgtaattggtaaagtgaaagatggttaaagaaggttagattgaccaaagcgagaatttaccttttttttttttttttttttgagacagagtctcatgccgttgcccaggctggagtgcagtggcgtgatcttggctcaccgtaacctccacttcctgggttcaagcagttctcccacctgagcctcccgagtagctgggtgacatgcgccaccacgctcagctaatttctttgtatttttagtagagactgggtttctccatgttggtcaggctggtctcgaactcctgacctcagtgatctgcccgccttggcctcccaaaatgctgggattacaggcatgagccactgtgtccggctgagagtgtacctttttttttttttatcaagcaatctagtacttgatcctaataatctttgtggtaggtgtttgcatttttagatgaggaaaagggaaatctatgagtcctaggaaatacagttggtatatgggaactgatatgtaattagacttaagtgatccatgttgaatttatgacttaagcacttaactataatcttaacctctccagttgtctgatgaagttagtatatgggaactgatacatagacttaagtgatccatgttgaatttatgacttcagcacttaactataattttaccctctccagttgtctgatgataataaaaacttgaagcagttatccatatggggatctctttggggaatcccagtcaccaaaagttaggttttctttaatattttttcatggaagatttcaaatatactcaaaattgaaagaattatataataaattctcatgagcccatcacacatcaataatgaatgtacagcattgcagtgtggagcttggcctattgctgaccactcagcaatgtggcagaaccactccatgattccccatggaaatgggaactacttcggttgtccttttatagaaaaattcagtaagtatctgctgattgtgccctacttgtgacttgaagccaggtttttttttttgttttttattttttttgttttgttttgtttgtaacagtcttgctctgtcatccaagaggggcatgatattggtgcactgcaacctccacctcctgggttcaagtgattctcgtgcctcagcctcccgagtagctgggactatgggcgtgcaccaccacacctggctaatttttgtatttagtagagatggagtttcatcatgttgcccaggctgctctcgaactcctgagctcaagcaatctacccacctccacctcccaaagtgctaagattacaggcatgagccaccatgacagcaaagctgggtatttcttaaattggttcagtcaggtgcaataaattatttgccctactctaaaatttaaaaatcttctaagaattatggttttgcagctgaggtttttttaagactcgagctccctggactcctacatatatccttagaacaacattgtccaatagaagtacaatgtgagccacatgttttgtttaacccagcatatccaaaatattaccccctttgcatgtgcttaatataaaaatttaagatgatttatattccaggttttcaatattcagtgagtaattttacacttagagcaagtatcatttcagactagtcacattttgagtactcaataaccacatatggctagtggctaccttactagatagcatagcctttgagtcccacaaagtgttcacattctatgtgtttacacacatcttttgaagtgtcatgacagagccagataggatccagattttctttaaatctggtctctcttccggattcctagttgattacttctttgttgcttcttatagggatgtatcagaagtttaataatcttatgattattatgttaactgcctgaagtattaatggtagcaataaattgaactataattttaatttttcaagtaaattttcttatgtgatactaaatatttcacatatacatgtatcaggaagtaaatgggggtaatttagtaatgaatagtatataatagtttatgatggtatttttcttttttttttttagattgcagcaaaaattggaggtgatgcagggacatcactgaattcaaatgactatggttatgggggacaaaaaagacctttagaagatggaggtaagttatactctaagtattttaaattgtttttcagagtgtttagttgaagtgattctcggtatttttctgttattttattgagatattaacttttattataaggttgttaaaattgtaagctgtatattggcctaaaaggggggaaagaaaactagacaaggtaagtaaaatttgaaagaaattttttaaaaaattttttaaaaaaggaagttttgtcttaatagaaaacaatttattttccctcttttaggattgtgccagattgaaagtttgcacagacgtcttgttaataatattaaaaaacatataaattgcttagaagacacttcactggttttactcatacgtgaatggattttaatatgctgtattttcgtcatttttctatttccaattgcaccttaaaggttgaaattcctatagtttgctactctagtgtgttgcaggttataccattttttttttaatgttctttactttcagtacttttgtgtttcacgtttagctttaaacctgtggattaaaacagtggatttacagtgctatgtatttttaaaaatcgtaatcgttgaagcttctgaacttagaagtctgcatgtattttttgttttaggtttgatatatgagttttgatacattttctttttacccttttttttaaagggaggattctcactgaggctatagaatgtatttgtagcttttgaccaggagaacttggtttccttttatttaagtgtctttcatatttatagtgaggtttttaatgtagaaaaaaaatgagctaatgatgcttcagatgttgtatgtaatgtattctttttattttatgtgtagatggctcttggacaagtccgagcagtacaacacactgggagggaatgccctctccttttaaaggcaggaatttttatttattacctgtgttcagtatgtaaacgtgaaataaaccagtggattcttaaatggacacaaatatttcttggattatgtgtctgcgcatattttatttttgctgcacaacattctgatgtttaatcatttaagtttgaaggggggaggagaatgtagtactttgagctataggttgtctgttccaaggtatgcattgtattcatctgtgtaatggatttaggtgaaggtagtcatgtagttgctttgagattttatttttttgctaaagttttatgcagtgaaatgtttgtttataaataatagaacagtttaggttgagattgccttgtaatgttgtggggggtttttttgtttttttttttttgggcatagctttgtggtcactgtcagatacactttaatatgtcagatttttgtagtttgataggcttttctccccccagtcttcagttcctgaggtggaagcatcattagcctttagcatgtgatattttgctagtaatggacctaaagtacgttgtcttgtgtcattctaatgtgcttaacatacattaaggtcagtgatttcttaagaatcagataactattttaatgtctgtgcatcttttgaacgtgaagagaatgaagtatcgtttcttttttagattactgagttggggttgaattttggcagttttggttcaaatgataaaccataccttcagatatttcaataaatgtttatattgttatttattcttgtttgggaggggagaagctttgtacttaattggcaaaaaattaaaagacacttaattttgcagatcaaccagatgctaagaaagttgctcctcaaaatgactgtaagtattccttttaaactgggtcaaaagctaaagtaacaatttcaatgttaagattttgttcatattattgggtatctttgaagttagttggtttatgagtattttggatcagctagcctgaatttctttgtaaatatatacctttttctcctattttacaatctgtcccattaattgtggccgggtatatgtaaagattggttgctgaattattttacatatttaacaactccaattcttgatctatacttgtacaacttgaaaaaggaaattattttgttctgtgccattgctaatataatgtcttccctttcattggctctcttgcccctgtcaatgccaatataaatattgtgtaaaaaatttgactctcttcaaggtgttgtctgtgcattagggagagatttttgaatgtttgatgtgattgtgttgttaaatatataagtaaatttaaattttgaatttttgttttatttttatttaattttttgatagcttttggaacacagttaccaccgatgcatcagcagcaaaggtatagtcacaagattttcaaaaagtactctgcaagttttggttgagctgtatgtaaaaacacaaccacattggtgtatattgaatatgtgtctgtgtattttttggtgtacctagttcatatcactccccttgggaaggtaccataaagtgatgatttttcttttgagtgagaaaaatttgtgatttggagagataggtggaattaaccacattttagaagaacaggggtgaattagagtaactgttaagatgacattctctaaactccacttcaacttctttacagttaatgccttcagactgttccattcatcatcccttcttcacttgatgtgtcatcttaaatttcttaatttaactactcaagtaataagatcatattttttgacatgagtctgagcctagaaccttagtttaagccattgggagacattagacttccatttttattaatagattatcttttatttgtaaacaaagtatctttcattgaaggaaaatggtgctttctgttatttcttagcagatctgtaatgacagaagaatacaaagttccagatggaatggttggattcagtaagtaacttgatttttaaagttttgaaaacatgatcaaaacatactttagaatctttcaaccaaaaaaaaaatttttttttttctaactagtaattggcagaggaggtgaacagatctcacgcatacaacaggaatctggatgcaaaatacagatagctcctggtaatgttacattctcatggtattttcagtgtgactagaaaactagcttttttttttttttaagccttctagtaacaataatgctacttttaatcttttgacctgaagttatctgtttgttttaaattgtagacagtggtggccttccagaaaggtcctgtatgttaactggaacacctgaatctgtccagtaagtttgaaaaatcttaaaaatctacttaagtaacaacagcagaactctttgaattttgtctcttctctttgttactgctttattttacactgtggtttcgctgccaccttccctcaaagtcctccaactcctttgaagtttatgcctcatgcctttctcaggtggggttcatcatctgaatcattaaacacagaaaatggttaaaacaactccatatctactccagtctctacttgtaaagccacgtgtagcctggagaagaatgcacagtcaggtcgactggtgacacttaaaactcagacattaagctcaagtggactgttgtgttgcctgcatttccctagttccattcacttttccactcctctcccaggctctttaatactgtatttccccacctccaaatcttcagcatctaaccccacgctctcccacttaagcttatttactgagaaaatggaagcaaatgataagaagcttttctttttccctaccactaaacctaccagccttcatttttcctctgttcacatagtactcaggtaattgctttccttttgtgttcgagtgcctaaagccagccctttcttcctactgaaggttcagcttgcagttgtactttcttctgcattgttagttctccctcattactaggttttttcttttcttttttttgagacgaagtctcgctctgttgccaggctggagtgcaggggcacgatctcggctcactgcaacctccgcctcccgagtagctgggactaccggtgcatgccaccacacccagctaatttttgtatttttttagtagagacagggtttcaccatgttggccgggatggtctcaatctcttgacctcgtgatccacccacctcggcctcccaaagtcctgggattacaggcgtgagccacttcccccagcctgattttctttatggcactttccaaataatgtgttattcatttgacatgttatttttacttatttaatgaaatgaagccactctagcaggaaccctgtttctttgagtgctattaccccattacctagaatagcacctgcacatagttgatatttaaatatttgttgaatgaataattgtagcatatgagtaagcaaaatggtagtttaaaaatgtaaataaatcatttagttcttggaagaatcagtttaattctgagataactttagcattagagttctttcttggaaattttggactattcttaaaaataaaaattgtatatctagaaaatttttttgcataatctctcaatctttgacccttgatggcattttctttcagttaaaagtaaaagcattgttaaagttagcatcaaggcacctaatcctgaactgggataggaggagtacttggttatattgttttatatttctctatttgaataagcttgggtatgctacagcttactatttaaatattaatttgttaacaggtcagcaaaacggttactggaccagattgttgaaaaaggaagaccagctcctggcttccatcatggcgatggaccgggaaatgcagttcaagaaatcatgattccagctagcaaggcaggattagtcattggaaaagggggagaaactattaaacagcttcaggtattgttatttttgtgaaatggctacttttgatctgttttgatgcccatttttgtccacttccttttgttaatatatattatttctatgattgtaacaggaacgggctggagttaaaatggttatgattcaagacgggccgcagaacactggtgctgacaaacctcttaggattacaggagacccatataaagttcaagtaaacttaactttatactttataaagaaagagtgggttgaatggggttgggcaaaatatgcatgaataattaaaatgttttgagacatgctttctaaattagctaactttttctgctttagcaagccaaggaaatggtgttagagttaattcgtgatcaaggcggtttcagagaagttcggaatgagtatgggtcaagaataggaggaaatgaagggatagatgtaagtaaaaatacccattcagaaatggttgtatgctaattcataaatataatagtgttttctgttttgtgttaagtagctctaacattgttatccttttatttcacctttatactttagaatacagaattctatatatcttgttaccctatttactataaatatagaattatatgtacttttatgatttgaggcagattttcaggaaatggcgcttttttaaaatacttttttttactttaaaccctgagaagctagctttcttaatacttagtcttttttacataaggtccccattccaagatttgctgttggcattgtaataggaagaaatggagagatgatcaaaaaaatacaaaatgatgctggtgttcgcattcagtttaagccaggtgagtacatataataatcttgtaagtgttggcagcagtgagttttgacatacatttattgtttaattaattttgtttctttgttttgaagatgatgggacaacacccgaaaggatagcacaaataacaggacctccagaccgatgtcaacatgctgcagaaattattacagaccttcttcgaagtgttcaggtttgatagaaagttaacattttcattttttgtttttatggaaaagtattttccttcatgaaatctgaagttacctctatatcagagtctgcttgatgatgctttattaatggagaaagtttaaattgctttaaggtaaagatcttggagcaggaagaactactaccttaagtgctaccttatttactcttgtattttaaaaaatgttattacttattagggccagttcatctctacatttctgattcaggtatatgagagctgggaaaaataaacttaataattttatcatgaaacaaaagtatttctgtgctgactcttcgttcttgtctttccctctctataggctggtaatcctggtggacctggacctggtggtcgaggaagaggtagaggtcaaggcaactggaacatgggaccacctggtggactacaggaatttaattttattgtgccaactgggaaaactggattaataataggaaaaggcaatgtattttaaactcttaatgttttaacacattattcatttttctggacactttctgttgctgtcgtaaacaagtggcaatgctttttctctgaccgtattttagtagaaaagaattcttatgttaatatgtaacaagtaaaacataaatgagggatctcatgtatatttagagaaagagcaggattttaatcttactagcttctagagaaagcgaactaagagataattattagcataagaaatgtcttttgacccaaaaagtggtttgagtgtttttgtttgtgcattttggtttttcccgactcatattttaaaaatttgaatgtttataagtgtattagtttatatttacactgcttttaaaagcagttaattcaaatattttattataatcacattaagtttatgtttaaacatactaagtaaatgtaaatgtattttaagagaagcatgaaatgcttcctaaaatttgattttcagtgtagaatattaaatgaaaaatcttaatacaatattgtcaattaggatactgaccaaaccatatttttaatggcccatttaattgtgaccattttcttctaaatagctcctagtacacccttgaaacctttagagaaattactgtcttttgattttaggaggtgaaaccataaaaagcataagccagcagtctggtgcaagaatagaacttcagagaaatcctccaccaaatgcagatcctaatatgaagttatttacaattcgtggcactccacaacagatagactatgctcggcaactcatagaagaaaagattggtgtgagtatactttaaacttttaatttttagtgtagacccttagactgtagttaaattaagacgtttattcaaatacatcaaaggaaaatgtatcattactagtcagcatttatagatttcatgatatgtataatagatacaacgtgaagattttccagcaatgaaaataacctaattaaatgtgtagttacaggttttgagaacaaccttacatttgggtgtggctagataagaggagggtagtgttacctgtaggcatgatattagtgttggtgtaggattgtggaacatacttgaaggacatagttaacgggaattcattatttattaagattttactctactgaaccccagcgaggcaaacaagataaatcagatacatctgccactctacagtagaaattcataaatcctaggttttggactggctcacagatcatctgggggtattaaaatgtagactatttgggtgcttcccactcctacacataatatacagagacagacctgactcaaaatgtctgggatagggcccagcatctaattttacatagatgtttgggtgattctggtgcacagacaaatttggaaatttttcctccaggaaagttttctgttagaacaaaaaagtatgaaacgctttgactgcttttttgtaagtgaggcagacagtgtcttactggagtttttaacacaaagtgtgcagggggcatcttaaattattagaattgcctaggaaaaattatttttggtgtttgccatgctgtgaatggggtgcgtgcaaaacaaggttgacactgtttctgtcaacttgggaagacaaaaatagatgtacaaaagacgcttaaggaacgtcttacaaaatgtacacaaacattgtgagttctgttgtatggatagtttaatggttcaaaaaatgaagaggatgtttgtagagtaatgatagaggttgatgccatggcaaaaaaatgaatagcactcatcttggttttttattttacagtattagcctaacatgcatgcgtcaggatttctgttggattcatggaaaaacaagatagattgttttggagaagcaatttggtgtggtgattaagagcatggactctgtagtcgggttgcctcagttcagaattctgtgaatcatttactggttctgtgaccttggagaagttactcaggcttttctgtgccttggtttcctcctgtaaaatgaagataatggtatctacttcatagagttgtagggattaaatgaatgttcatgtgtgtgtcacttaaaagaatgcctgccacataaccctaaaaaatgttgctactttttcagtattatttttactacttggaaagaataggtcatggatagtaagtgagagatgacagcaattggagatttaaagagacaagttaataaaaagaactttaaaggtcatgaagtttagtttccctattttgcagtgagaaatttacaacaaaatgtagtgttagcattttgtccaacatgtcgcctggttgtgttaactactcagaaggagcattttaggacagttaagtgtaatgtctttgttggcttaaccaaacagaaatcagttaagcgttattaatgatgtggcatgcatgcatgataaggatataaaatatcctgatttattgaaggaattaaaaagggaatttttgtgctattaaacatcatgatacatgaaaggccaaaaaggatataaattattgatctgaatgggattttagtgacagaaataggttgtgaggtggattttagtttcatagtgaaacaactagctattaacgttatcagtgaaatgttcagaagacgatgatacaggacccaagcggtttgggaatatattgtcaagacggttgtctcatgttaggcaagtagatatagagagaagagctgaagataaggacctgatttctgtgagtaagatggaaaaagatagtaaaagtaagaatgataaaaggaaagggaactactgctgtccgcaaacaagctaaaggaaatttaattgctaatttaaatttaatttaatttaaattgcatttaattgctactgctattgattttagtgaattttacatgtctcattattatggcagatgaaatagtttttgcaaaatgaatgaggaaaggaaggaaaacctaaaatttgttatttgtgactataagagggtagaaatggatgattatttggccgtagagttgtttaaccattactgtgtttttctgatttttctatgtcatccttttttttgtagggcccagtaaatcctttagggccacctgtaccccatgggccccatggtgtcccaggcccccatggacctcctgggcctccagggcctggaactccaatgggaccatacaaccctgcaccttataatcctggaccaccaggcccggctcctcagtaagtattgggtttagttctgggctttccccaaagattctagttttgggactgttttttatgctgatttttcttttcagtggtcctccagccccatatgctccccagggatggggaaatgcatatccacactggcagcagcaggctcctcctgatccaggtagaagatgcttattatttgtgtgttatctgtattattttccactcctgttacattattaaattttctagtgttgattctacatttgtatgcatcaccttcactcactttactctttcaacagtgttaggcactgcctctaccccagtgtataggactgacatgaatatgagctctgcttttatggaatttctttctacttgcctttggcttatgagttgatacagtagaatgataaaagctaaaagctgcaggaaagagcacagtgtcataggttttggataccagtgctgtcaaatgtgtagtatgttcattgtgacattatctgtggaaaaatagtttttacttatttagaaaagtatgtgataggggctgggcactgaggctcacgtctgtaatcccacctattcagaagtctgaggctgaggcttgagaccagcctgggcaatatggcaagacccatctctaaaaaaatttgttttaaattagccgggcagggtggtatgtgcctgtacttgaggctgaggtgggaggattgcttgagcctagaagttctagattgtattgagctgtgggcacaccattgcactcccctgggcaacagaatgggatcccatctcttaaaaaattatgtatatatgtaacagtctatataaatatatatatataacagtccaacagagtgttaagtattggggcattaaacttccaaattgtcaaataagatatctgttctagtactcctcatatgacaacttcatgtgagtaaaaatcaggcctgtatttaataactgcatgctaaagcccaaatacgtttaattattttctatattcagatttatttttatcctattatattctggcatctttccataccctgtagtttgctttccatcttggtcaaagagtcattctttgaaaccaattaacatttatttatgatccttttttctcacctgctacctaccttttaggcctctttcactcgtgtagtttcaaggaaaatatactcaattatggaatactttctacacataatacatttatcccaaaaaaacttgaagtaatttatgtaaatgaaattgcttgattaacttcataggaagtgtgctgtttggaatatgatgacacagcatgacagttaccaagcatgacttgagttgtgctaggatataggtgtgcaaagttggggttgtgttctatagcaaaagaatgcactcccagcgtaagcaacactgatggaaggggctcacagggtacaggatataatactcttaacaactaatttttggagaatgaaagggcttttctttccctcttgttggctgattgggatggtataattaatgggattgaagagtttgagtaggttaaaaggcagattcatattgggtaacttggatctgccaggattgtatttttgagcactactgggtggttagcatgattgaggaaaaatgatgggaataagaagtggaagtggtctttgtatcacaagttgaatttctcacttggagtagtagtgaagtcactactgtaagagctggtcagtgaatgtggttgcagcatggcctttgggcaagaagtaacccatttaactaaaaccagctggttggccccactcagatttatcaaagggttactgggtccctgggggtggatattgcttatattagacttagaatagcatactgttttaatattatatgaactaaaatgtttctttaaaaaaagagtggtctgttaatggatttatgtagtggtcaagaatttagacttcagagtcaaataaacctatatcagtcctagtcctacagtttactaattgtgagatgtcaagcaagtttttgaactcctctaagcctctgttttcttatctataaattaataaatgaatgaatcgggttgagtgaatatttagtaaattcttagtacatactagttatttgtaactgtgagactggttttttggtatggttttcacatttgggagtagaaataccacttcctaaagtctgttttatctcaaattctctatccaggcatagtgtaaagtgaaatacctagatttcttgattaatatacagataatggccagacgccatggctaaaacctgtgacgctagcacttcggaaggctgaggcgggcggatcacttgaggtcaggagttggagaccagcctggcaaacatggcgaaaccctgtctctactaaaaatacaaaaattagctggatgtggtggcaggtgtctgtaatcccagctacttaggaggctgagacaggagaactccttgagaattgctccactgccctccagcctgggcaacagagtgagacacttcatctcaaaaaaaaaaaaaaaatacagataatgacactattgagatatgtaaacatccaacataaaaaagcagtacattgggcaattgagaaaagtttggcaggtgtcctaataacaccttacaattatattaccttgcaattttctttttctagaaattcttgctcatttttctcaaacttgactaacctttattaagcagctgagaattgctactgttcagaatgaaggtataatagaaaatttaaaagttttaattgtatggtattcctagtataaaagacaatcaagttttttctggtttctagaagattgaaaggtctcaaactgttcgcacttcagttgatgtgggagatgagtgagggtcagtcaagtgtagaggagacaacatagctaaaagccgagacacgggcatagtgatttctgaaaagtacaagcactgtgttgtggctggagcttgggtgttgaagagatacaaccaaagggagagatgaggctgaaatggaaaggataagccaggggcttcaggctgttaaagatgttgaatgataggctgtacttaagctttgaacatcctgaagattgcagagaatgaagaatttcaagtaggatgacatcaagtttataacattcaatgtgaaataggatgaaatgtggctaatcttttttaagatttttatattttctcttcattgaaaataaggacaaagttcattgttctaaaataattttctttcttatacaggttagtccctagaaatgtttttctttagtcatcttctagatagagctgtttgtgcttgaggcgaaaccaatttagaaaaaaacaagggcacaggatggtttgagacagagcattggatttggagccaaaagacctttattcatatcccagttatgccactcagtagtttttaacctaggaaagtcacttagtctctgagacttggttttctttaataaaagctgatggtgacaacaaaaataatagaaaatttaagttttcatatactttttttaatgttctagttaattttgggggctgtatatttgccagagagctgggcgggggggtggtgtgtgtgtgtgtgagagagaattacagacttcacatgcaaaccttgaactttcatttattttagctaaggcaggaacggatccaaattcagcagcttgggctgcttattacgctcactattatcaacagcaagcacagccaccaccagcagcccctgcaggtgcaccaactacaactcaaactaatggacaaggtaactacagaacttattgtatgtgaaagccaaaagttgtgcttggaattatatatgaagtacatcactgtataatacctaaaatttctgacattatttaattataatttaagcagacttttccttttttaaattgttactttgaaatagtttcaaactttcataaaagttgtaagaagaatacccggagcacccagatagcccagctgttaatgttttattcccttgtttactctgttaccatctatatcatacatgtgtatctgtatttattttaaatatccattataattagttctgaaggaagctaattatatccagcataattagttctgaaagaaatcactgaagactaaactgtagacatgatgtcctgttgggtacttcttcaaaaatacataaccatcatacagccctccaaatcagtaagtcaacattgatatattaagttctttatatcattatggatttctgatttttgcagtaggttatatttttaaagtatttttataattttgataatcagattatcctggatttggccagtcagggagtatattcagggtggtgcctatatccttttgaaatacctgtcattcttttaagcacttccatactgtctggcacagtaagattgttattttgtgctttctgggctccagccctcgagtcagctatttcttcaaggagctcttattccttttagtagagtatggtagttagaaacgagacttgagtatgcttgttgctactgaggtgtaattgcttctagcttctttcaacaggcagaactaggaaatatatttacatacatgcatacctacatacacacaccaaaaaacacataaacatcgatatatgtatatatttttaaaaactatgttcatatcaccacatccgtttcagcattttggggttttcaagccttttccttttttgtacttgtttacaaacgtgagaaacctggtgtcctcagtgtatttccttatttgacatgcatttacttatatgttcaacataaccagtcttcaaacaggttggttttcttttctgtccaccacctctgtacccccagtaccttctatctttggcactgttagggatgccaccaccacatagtacttccctcctaccctcactgtcacattgcaggcccctgccagctcctgcacccaaggaaacggccaaaattgccttttaaaaactttaattctgttttttgttttgttttgcttgaacaaggagatcagcagaatccagccccagctggacaggttgattataccaaggcttgggaagagtactacaagaaaatgggtatgttttatacatttcttgaaaatacatacttaattaaattgaaacaaattattctcttcaggaagagaataattgaataaaatcactggacttgtaaacatatcaagacagttgtaaattatagtttttaaatttgtggttatatggcaaggaaatttttttttctaattgcatttgtcaaccagttattaattgaaactagaaatgtccttactggtacatacaatattaacattactatacttttgacaatgacagttatatatattatcagtctaaatgacataaggttaaattttaatgtgtcaggcgaaaattgtgtgtgataccattatttttgctgcaagataagcaggtaagaagtaatctgcagtgacggaaagtaaccaagtgatggaaccagaatctggcttccaagagggtctgagtcccaagcttgtctcccaaatttgtctctttaggaaccatttggaacctgatactatacttctggacaaatcactatatttcagctgcttttgctcttagtcatttaaaattattacataccacagctagatgtcacaaatgaaagctaaattggtaagcttggttatccttcactagcagaaaaagaacctataggtggtagagttttgtcataagagaacggtctacttgggattttcaatgttttctttgccaaagaatgtttctcattctccacagaaagaaaaatttccagaaaggtgatgattttaatcttctagatgtaaaattacatatacctgatgataaagttgttttgcacaactggtttctttttaaagaaaaattgtttttcctcttagaatggcttcctaggagagtcatgttccgtctctttctgaggctttaacagattatgtttttgtgacctagcttaggcagatctacagtagctacaattcggcaaaaagaaacttttaacttaaaaacagcatactctgattaaggttggttacataatttattttctgaactgggatcctttttagaatgaatagggatgctattaataatcatgccctgacagcattgtggtcaggacctgtaatcaacttaactttaattaaatagcatcaccattttaaaagacttgagcatgagccacgtgcggtggcacacacttgtagtcccagctgcttgggaggctaaggtggaaggattccttgaacacaggagattgaggccagccttggcatcatagtgagacctggtcacgtgtttgggttaaaaatcactaacttcaacttctattttctcaatgggtaatgtcccctagatagggtccctagttattattaagtaggtaaaaataaaggcttgttaatggatttaggtaattatggaggaatgagtttggcttctgtgctttattttaccatattgattatttgtaatatggccattaatacatttactgtttagtcttttttgttttactttttatgttttactcaaaatgagtgggtgggtggaattctaatttttattgttaagggaagacattttaccttgtctttaattttttatttttttttaccatttccctgccagttagagatactatactatactgtcttgaatcctctgtaggaaaacatggcatagaaataattaaataataattagatgttaaataataatgctgtatgactaaagaacctccttaccccaccttttctgttgttgttctgtagtaccacaataatcacttgttaattttatttatttatttatttgagagggagtctcactctcgcccaggctggagtgcggtggcgcaatctcagctcactgcaacctccgcctcccaggttcaagcgattctccggcctcatactcccaagtagctgggattacaggcgcctgctaccacatccggctgattttttgtatatttagtagatacgggctttcatcatgttggccaagctggtctcaaactcctgacctcaggtgatccacctgcctcagccttccaaagtgttgggattatgggtgtgagccactgcacccagcctcacctgttaattttatgagcaaaacagattagttgggcaagtccttcacatgcatatctcgttgttgttgttttttttaagacggagtcttggtcttactccccaagcaatggtgcgatttcgtctcagtgcaacctctgcttcctgggttcaagctattctcctgcctcaacttcccaagtagctgggattacaggcgcccaaccacacccagctaatttttgtatttttagtagagacagagtttcaccatgttggccaggctggtctcgaactcctgacctcaggtgatccacccgcctcagcctcgcaaagtgttgggattacaggtgtgagccactgcgcctggccacatgtgtatatcttaaaggaaataatgctaggtaatttagtcaggtgcttgatgagcatttgtcatcatcatacggagtcaatttgtctttttctataaaagttctttttgtaaatgattagttgcttaactgctttaatttcttctaggtaccttacctgtcatcaggatctttttaccacaaataagaaaccttaaagcataaaacttggttcagtcttcacatttcttaaatggaaggagaaagggaataatagggagatatctttatctcaaagccaactgttgttgacttttccagtggcaggggtatgatgctaggacttcagatttcctgattccccatcctagtgccccttttgccaaactaggcaggctttcacagcttttggagcctaatttaagtttttcttgttaagaacagaaaactccattcatagattttaatttgtcattatttgcttatttactgaaaaaaaaaaaaaactattgaaaacaggtgtgggttaagtttcccaaaattaggtttatatttcaaaacaattctaagttcccaaggataacaccaaactaagaggataaatttttattttatttttttttattttatttttttgagatggagcctttccctgtcgcccaggctgaagtgcagtggcatgatctcggctcacttgcagcatccacctcccaggttgaagcaattcttctgcctcagcctcccaagtagagaaaatcttgataagatttcagaggcttataaggaacagcagtaaagatgggtgtttttaaagctagaatacagggatttttttaagacccttaagagaacaaacagggtttcaaaatagaaaggagtaattcttgtttggagaagtgaagacatttgggaaaagtttgacttgaggaaggccattatgataaggactgatgtggtagactctgaaccctgaagaatctggtaccttaagcctaaaggagggtagagtgaggacttgtgggaaaggtacagttatatgggaagacaactgagtgtgaaaatctattcatgcaaggctggctcttaggctctaatagaatcttgcaagctaactctagagcaagtcttttatctttgggatgaaagaaatttaaggctaaactttatacattataattataaaactataataaatattagataatgttctactgaagtataaactcaactatctggcatcaaacagtaactaagccatgatcacaccactgccactgtgttctggcctaggtgacagagcaagaccctgtctcaaaacaaaatccagtaactaaagagaggaataaggggagcgcaaggtaaggcagtacatgtctgaaggggcagggaaagagttctttctctacttccaactgggcaaaataaatcacatttgccctttagattggaagagtgaaataagcttttcttgaacatcttttaagattggagtgtcaaatattaccaacttatttaccaagaatttggtttttcttaaagtctaaagtgtttactattagctttccacagggatataggagtttgcagaggttgtagttttttaagggaacatcaatgaattttcttgatgcatatgcctgtttctaccattttacattgttaatttgcctctaaaatgagtaactcttacaatggggtttaaaacctgaagactattgattgctaccttgatcaggtttggtttcaagtgtgcaccctgtaagagacaatgtgtggttttattgctattgtcacaccagttttttagatattgaaacctgtttcagatttgcttgaattgtgtcttgtaagaggaaaatgttaaacttacttcctctttgagaacagttatttatagaagacaggaaaatatgagaatttttaatagtatatgagagttctctgttacccaagaaaagagggtttttttcaggcatttttaaagaatcataaatcttaaattctttcactcagttgctttgagtctgtgacctgttttacaatggtgatagactgctttctgaaactatgaaattggtcttgttggcagcatcctacaaacataaaaagagcttcctgtgtgtccatgctccagtatttttgtctatggtagttatttcacaaagccaagcgagttacaaacgaaataaaatagtgcttaagtaaagaaactgaataggagaacatatactctctcttctcaattttttatttagaaaattatataccttcagaaattggggaaataaggcatcagacactcagcttcatcattttttaaaagtttgctgtatgcgctttatatgtatgttttttgtgtatgaaccatttcaaagtaagttgcagacaagacagtttgctctaaataaactcaatgtctgggcacagtggttgacgcctgtaatcccaggactttgggaggccaaggcaggtggatcacttgagtccaggagttcaggaccagcctggacaacatggcaaaaccccatctctacaaaaaaagagaaaaatgtcagctaggcatggtgggcctgtagtcccagctactctagaggctgaggtgggaggatcacctgagccctgggaggttgaggctgcagtgagccatgattgcaccgctgcactccagcctgggtggcagagtgagaccctgtctgaaaataaataccctcaatattatcacaaacatacagactatatttaaatttccatagttgtcaggaatatgtcttttgtcgtctgtgtgttttgtttaattcagagtacattcaagattcatgcgttgcatttcattgctatatttctttaatctcttaattcagcaaagtcaccttatttggaaaaagacctgattgtgaaagctgagttttatgtgttttcattgatcttgttctttattccttgtcaggttacttttaataacattgtgttgtaattggaataaattattaatacttttaacatcttattatgtgtgtgtgtacatacactttataggttactaatcaggaaaaagtcttggctaggtcttaatacagtctttaaatcattgcctttaagtgggcttaaagtttttcaaaaatgttctttttgtaattctggaatcgaattaagattatgcctaaatctttaccttccttagctaaagcagtgtggatttggggttgattctgtttttttactaataatgacgctctagaactaaaagttaacgattaattataaggcaaaaagaaaagagcgttctttttttttttctaattgcagagtagtttcctgacactactaaatgaatattttaaataaaacaggagtaattctgaccctctgtgcttttgtcttataacctgtacttacagtggatgtaattttatattaaagtttaggggtttttttttttggtcaacaagggcaaacacaagattattcaaaggcttgggagaaatattacaagaagcaaggtattgtttttattagaaatgagatgttgggcttataattgtggttacagcaacaaagttcttttttttcaaaggtcaggcagttcctgctccgactggggctcctccaggtggtcagccagattatagtgcagcctgggctgagtattatagacaacaagcagcctattatgcccagacaagtccccagggaatgccacagcatcctccagcacctcaggtataatgtaattgctaatttgttgatttctactccagtctgttttctgcatgtttactgtttgtctgtttgggagtgtttgccttttaaatttttatctggcaaagtataataactatttaaatgaagtactacggtgtattgtttgggtttttttgttttttataatgctttccagcatctgagtggtgaatatttctgcaatgcctttgattttaaaaataaattttcttcccccagggatttgcaaatcatgcaagaagccaccaccatttatattaaccactttttctttcttaaaggattcactcctgaattagctccatttcaaggattttctttaactttttgtgtatttcttatgtatctcttctgcacagggccaataataagaagtggacaatacagtatttgcttcattgtgtgggggaaaaaaacctttgttaaatatatggatgcagacgacttgatgaagatcttaattttgtttttggtttaaaatagtgtttccttttttttttttttttttgaaaatgtacaaaatatctatcactactgataggaggttaatatttctgtgtagaaatgaaaattggtttgtttttagtatttagtgtagatgtacacattccagcaaatgtatttgcaattatgtggttgatgctttgtgatataaatgtactttttcaatgtatactttcacttttaaaatgcctgttttgtgctttacaataaatgatatgaaacctcctgtgtcggtaagttggatatgtgggtatttaaaggattcataatttcttagcaatgataaattaagatacatatacacaaatatataagctttccccatgaaatattgagtttttaaacactggcatgtttttccccccttgcagtatagtggtagattggaggatcttttccatttattgtattggctctttcagcacaagtaatcctgatatcttcattttttttccttctgtttgattaaaaactgcatgtgtgtacaatgatcttttggcatacttccattgcattaacagtgaaatttccttttatacatgaccactgtttcagacctgtactgctgctataacagttaacctttctgttcttaatttgataatacttgatttccaagactgtttcggcataactaattttaaacagttttcagatagtgaatatgagtagtctaataagaacagtttttttccatgtgaagcaactctttcaatgtatataatgttagtgtgtttctttctaaatttaggatagaaaagtgaatagtgtgcaaaaagtatagctacattgcatctgccattgaaacataaatggggtatggaaacgttcaagctttttttttttctttatgcagtatagataagctttgttttgtaaatgcacaagtccaatcattgaatcaacttaatttttttatgtacttgaagtcattttattactctttaacactcatgctgaagttctgatattttgttgaaatccattgttttactctttgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagaatgacaaattgatgaactgcaacagtatctttttgtcaatgttccacataatgcaaatgccatacgttgtgtgaatattatgttggaatacagtgctgatatcttggaaaaccataactgcctcttaatttaacatagaataatacatagttctgtattttttttaaagtgagcttaatgggtaagtattttttatatgctttagctatagctaaagaaaactgatacttaacaaagttgaatagtattattcactggtgctcctaaaatattgtttttcagtgtaaaatatgcatatcttctatatttaatatgaaagtcttgaaatgtatcagacagaaggggatttcagtttgcaaataatgagcaatgtagcaattttaacacatttcataaatatatattttgtcattggtggagagcaccatttgttgttttgaatatactttaaaggaagaggtacaaggacataaatgttgagattacctacaggatggaaatagcagtacagttcattgtagatattttgaaatgtttttgattgttttatataacctagagtgacttcccttacccttatttagatctgcatatatagttctagtatgaagtttaatagttaaggagttagctatttgttatctttaagagtagggtattgacgtgaacaattgcagtattttgcatgatactgttttatagatgaccttttaggaaagtggtgcatttattaattgaactgaagaagtagttcagttgaattcagtatcataattcacaaattggaggctgttgattttgattcatttaaggtttaaaatctttattaattgcaaacagtgcaattatttatacttcacagtgccttcccagaccttccaccttaggttctgctgcaaaaagcaccaggtaagcacaacctaaggacatatataaataaatatttcaatacattaatgttgtccctgtgaggtttttgtggttgtgtattcaaaggcaatctgctactgcttccccaaaatgtattttgttattttatgctaccatcttagtggaaagtctgtaagttgttaaagcaactgtttacatttctgggtaatgttttttattttacttttttttttttattaagacaagaaaatgatgagtagattgctgcagtaattgaactacatccaaatctttttgtattttttccccaaatatagaagtgttaatattaagaaaggacaattacacagttttcaagatttaggaaatcacttgtttagaaacttcaacagccttcacaatctgttttatatgatggacagaaaatttctttgccctccaaaattataatttctttatttttttcttattcttaaactataataattcagtaaggatattatgggttagaattttattatgatttttttcttagacaaaagttatatgctgaagaaggaaaaagttataaggcagtatgttttgataaaaggcatgtgcatcagtgaaatgttaactgtatagcaaataacctttcataatctgtagcaatcagtatttttctgatttaataatattttaataactgacgctgcatttaatttttttgccagtttaaaatgtttgtgtgtttttatagatgattttaactggtacatattttgagttaagttgaatgtatgaaagcagcatcttatcagttttgtttattcgatttctaaaatgtgctgatccttttaaaactcctgcttatctctgcaacaaagaaaaatattcaaaaatactgccttcattttcacacacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgaaatgtattcccatacataatatggtatagggtgtaatgtacctgcttttgatcacttttcattttaaagtgctattcacttgatcttaaatgttccatgaactgttaaatttcttaagttacatagttactacaccacatttatgtgtatgttatgttttaatagtcaatgataggtatgtacaattgataatataaaggggctcattgaaacttgagagcctgttgagttttggttagttgtagattgcatttttataaaaaaaaatacagatagattgatgataatagatattggggcattgtttctgtctcatgagaattcttttattcattaccataagccttcactgatactataagcattattttaaatgacgctgatcttaagtctgaaataaatggaaagcagaaaaggtgagccagttgatttgaatgcattggatattagtgttagaaacaatgtatagtttagattgaaactgaactgacttatttagcacttaaacaaaaatttgacaatgtttttagttttttttaagacagcttagtgtggtgatacttagaattctatggtttgatgtttcttttagaaatgagaagtatagttttattttttaatataaaaaatggttttaatactaaaactagtaatttgatactagttgtttataaacattgtaaaatatatcttttaaacaaattatcttggtagttaattcataagggtgggtttgggtaggaatagcagagtactttcagagggaaaggggagtcattcagaagtgatagcattttatttgtttgaatactctgccagtaaaatcagctgtacttagaaagttatctgttgtgtagaataatgatgtagagtttactaatcagtgaggatgtcttgtttttattttctgcaaactctgcctcactttaaaatgcattataacaatacctaattaaagataattttggctctgaaagttaccttattttttgttgagttagtgacttcatttttcttgccacaatataagcttttgagggatttttttaaattggtgcttttaataagcaaataaatcccagggttttattttcttcagtgatacccctatagaaactcttaaatgtatttgcgcatatatatatatatattttcttatgcatgctcgatgcattttcgtcctgagaaaaatgttctctacagaaactacccgtgtgtaaaaagaagattggcttaaaatggctactgtgatgggaacagtgtcttagggagatgcagcttggacttgaggtaaattgaatactttacaactgtggtttagagtttgctttaatgacattgtatgtaaaaggtcacatgattgctgtaattttgtattcattatggtttcctcaataaatgtacattgatgactatta

SEQ ID NO: 2 - DNA, 696 bp, Homo sapiens
  source 1..696: mol_type = genomic DNA
atggcagactattcaacagtgcctcccccctcttctggctcagctggtggcggtggtggcggcggtggtggtggaggagttaacgacgctttcaaagatgcactgcagagagcccggcagattgcagcaaaaattggaggtgatgcagggacatcactgaattcaaatgactatggttatgggggacaaaaaagacctttagaagatggagatggctcttggacaagtccgagcagtacaacacactgggagggaatgccctctccttttaaagatcaaccagatgctaagaaagttgctcctcaaaatgactcttttggaacacagttaccaccgatgcatcagcagcaaagatctgtaatgacagaagaatacaaagttccagatggaatggttggattcataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattccagctagcaaggcaggattagtcattggaaaagggggagaaactattaaacagcttcag

SEQ ID NO: 3 - DNA, 1968 bp, Homo sapiens
  source 1..1968: mol_type = genomic DNA
atggcagactattcaacagtgcctcccccctcttctggctcagctggtggcggtggtggcggcggtggtggtggaggagttaacgacgctttcaaagatgcactgcagagagcccggcagattgcagcaaaaattggaggtgatgcagggacatcactgaattcaaatgactatggttatgggggacaaaaaagacctttagaagatggagatcaaccagatgctaagaaagttgctcctcaaaatgactcttttggaacacagttaccaccgatgcatcagcagcaaagcagatctgtaatgacagaagaatacaaagttccagatggaatggttggattcataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattccagctagcaaggcaggattagtcattggaaaagggggagaaactattaaacagcttcaggaacgggctggagttaaaatggttatgattcaagacgggccgcagaacactggtgctgacaaacctcttaggattacaggagacccatataaagttcaacaagccaaggaaatggtgttagagttaattcgtgatcaaggcggtttcagagaagttcggaatgagtatgggtcaagaataggaggaaatgaagggatagatgtccccattccaagatttgctgttggcattgtaataggaagaaatggagagatgatcaaaaaaatacaaaatgatgctggtgttcgcattcagtttaagccagatgatgggacaacacccgaaaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccagcagtctggtgcaagaatagaacttcagagaaatcctccaccaaatgcagatcctaatatgaagttatttacaattcgtggcactccacaacagatagactatgctcggcaactcatagaagaaaagattggtggcccagtaaatcctttagggccacctgtaccccatgggccccatggtgtcccaggcccccatggacctcctgggcctccagggcctggaactccaatgggaccatacaaccctgcaccttataatcctggaccaccaggcccggctcctcatggtcctccagccccatatgctccccagggatggggaaatgcatatccacactggcagcagcaggctcctcctgatccagctaaggcaggaacggatccaaattcagcagcttgggctgcttattacgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaggcagttcctgctccgactggggctcctccaggtggtcagccagattatagtgcagcctgggctgagtattatagacaacaagcagcctattatgcccagacaagtccccagggaatgccacagcatcctccagcacctcagggatttgcaaatcatgcaagaagccaccaccatttatattaa

SEQ ID NO: 4 - DNA, 1935 bp, Homo sapiens
  source 1..1935: mol_type = genomic DNA
atggcagactattcaacagtgcctcccccctcttctggctcagctggtggcggtggtggcggcggtggtggtggaggagttaacgacgctttcaaagatgcactgcagagagcccggcagattgcagcaaaaattggaggtgatgcagggacatcactgaattcaaatgactatggttatgggggacaaaaaagacctttagaagatggagatcaaccagatgctaagaaagttgctcctcaaaatgactcttttggaacacagttaccaccgatgcatcagcagcaaagcagatctgtaatgacagaagaatacaaagttccagatggaatggttggattcataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattccagctagcaaggcaggattagtcattggaaaagggggagaaactattaaacagcttcaggaacgggctggagttaaaatggttatgattcaagacgggccgcagaacactggtgctgacaaacctcttaggattacaggagacccatataaagttcaacaagccaaggaaatggtgttagagttaattcgtgatcaaggcggtttcagagaagttcggaatgagtatgggtcaagaataggaggaaatgaagggatagatgtccccattccaagatttgctgttggcattgtaataggaagaaatggagagatgatcaaaaaaatacaaaatgatgctggtgttcgcattcagtttaagccagatgatgggacaacacccgaaaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccagcagtctggtgcaagaatagaacttcagagaaatcctccaccaaatgcagatcctaatatgaagttatttacaattcgtggcactccacaacagatagactatgctcggcaactcatagaagaaaagattggtggcccagtaaatcctttagggccacctgtaccccatgggccccatggtgtcccaggcccccatggacctcctgggcctccagggcctggaactccaatgggaccatacaaccctgcaccttataatcctggaccaccaggcccggctcctcatggtcctccagccccatatgctccccagggatggggaaatgcatatccacactggcagcagcaggctcctcctgatccagctaaggcaggaacggatccaaattcagcagcttgggctgcttattacgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaggcagttcctgctccgactggggctcctccaggtggtcagccagattatagtgcagcctgggctgagtattatagacaacaagcagcctattatgcccagacaagtccccagggaatgccacagcatcctccagcacctcagggccaataa

SEQ ID NO: 5 - DNA, 39750 bp, Macaca fascicularis
  misc_feature 1246..1266: note = n is a, c, g, or t
  misc_feature 2077..2131: note = n is a, c, g, or t
  misc_feature 2666..2690: note = n is a, c, g, or t
  misc_feature 3304..3345: note = n is a, c, g, or t
  misc_feature 3869..3870: note = n is a, c, g, or t
  misc_feature 6076: note = n is a, c, g, or t
  misc_feature 6091..6093: note = n is a, c, g, or t
  misc_feature 6159..6183: note = n is a, c, g, or t
  misc_feature 6259..6277: note = n is a, c, g, or t
  misc_feature 6869..6906: note = n is a, c, g, or t
  misc_feature 7072..7139: note = n is a, c, g, or t
  misc_feature 7714: note = n is a, c, g, or t
  misc_feature 11374..11394: note = n is a, c, g, or t
  misc_feature 11454..11491: note = n is a, c, g, or t
  misc_feature 12562: note = n is a, c, g, or t
  misc_feature 12699..12742: note = n is a, c, g, or t
  misc_feature 12793..12794: note = n is a, c, g, or t
  misc_feature 13648..13656: note = n is a, c, g, or t
  misc_feature 14247..14261: note = n is a, c, g, or t
  misc_feature 14474..20234: note = n is a, c, g, or t
  misc_feature 21758..21789: note = n is a, c, g, or t
  misc_feature 23223..23225: note = n is a, c, g, or t
  misc_feature 24454..24512: note = n is a, c, g, or t
  misc_feature 24563..24589: note = n is a, c, g, or t
  misc_feature 25270..25338: note = n is a, c, g, or t
  misc_feature 25723..25734: note = n is a, c, g, or t
  misc_feature 25857: note = n is a, c, g, or t
  misc_feature 26517..26523: note = n is a, c, g, or t
  misc_feature 27589..27590: note = n is a, c, g, or t
  misc_feature 27660..27688: note = n is a, c, g, or t
  misc_feature 28733: note = n is a, c, g, or t
  misc_feature 30266: note = n is a, c, g, or t
  misc_feature 30317..30371: note = n is a, c, g, or t
  misc_feature 30453..30768: note = n is a, c, g, or t
  misc_feature 30833..30837: note = n is a, c, g, or t
  misc_feature 30906..30912: note = n is a, c, g, or t
  misc_feature 30997..30998: note = n is a, c, g, or t
  misc_feature 31271..31562: note = n is a, c, g, or t
  misc_feature 31697: note = n is a, c, g, or t
  misc_feature 31707..31782: note = n is a, c, g, or t
  misc_feature 32257: note = n is a, c, g, or t
  misc_feature 33477..33478: note = n is a, c, g, or t
  misc_feature 37353..37427: note = n is a, c, g, or t
  source 1..39750: mol_type = genomic DNA
gagatttcgttccttcagtctccacccctttacggcacgatggtcgcgcaagaatgtgatacagcttcgacggccgccattttctttctttcttagctgttagctgagaggaagtctctgaacgggcggcagcggctagcgtagtgtaaccatggcagactattcaacagtgcctccaccgtcttctggctcagctggtggcggcggtggcggcggtggtggtggaggagttaacgacgctttcaaagatgcactacagagagcccggcaggtaagtgtggaccgcgcggcggaatcccgaaagctcacggtaattggccgctgactgagtaggccgctaccctttagcgcatgaggaagaggaaagaggtgtccttccgggctgaaatgtgaggagacacgtttccccttgttggtaataaagattagagaccagaactcagttttgtgttcttggtgtgtaatccacttagaaccccgacgcgtgctacgcaaaggcctgaagtctttctcccgcttctgcggcactcgtgtgtcgccagcgagctagcttagcctccccttttcctcgagatgaagatcctcttccaggggataaagcgcaggtagtttcacacaatttaatggaaggttctggtaatcagtttgggaaagaactagggtcggtctcctggagccatagcaagggaagggatttgtcgttaaagtagcctttacagctcatttccgttccctctcgcaattaaaaccgctttccgtacctttcaccttctcacctctacaaggaagggacttgaaagccgtctttttctgggcgggatttacgcgtcagtctgttctaacagtcagtcccccttatttctcaaaatggcctcaggcccattataccagaggtttcaatttgaatctgcctctctgttaagagtcgtaaactgaccagacctctttgtattacgtagtgcgtacatttgccctgaagacaccactttcccagacgaaagctgttaaaatagtgcgagtattccaggaaaatagaagatttcttaatttgaactttacatttttagatagtcccctaatatatttaaaatttcaaaatgtatggtgttggtatggatttgcatgtaagcaaaagaattctattctctatgacactatgcatgttgtactaggtgctggacatttttactagtgttaagctaatggtaggtagaaaccagtgttgtgctgtgttttcattgcattnnnnnnnnnnnnnnnnnnnnncggtacagttaaaggaacattgagtcaaaatcagaattcataaaatccgttgtaacatacctaatgtgaacacattatcaacatgtacctgtacttgtttataaccagagatattagaggtagtcaacaaaaggttatcattaatattagttctgcggttttttcacataatttaagaaattctgaacatgtttagcagcaagcatttgtataactatgacaaacactgttagcgcgttttattagattgatttgtaaaacttaactattgtatttattggaaccaactattttattttactagaaccagcacattggattacttagtatcaaacactgtaggtagatactggagttttgttttgttttgtttgagacggagtctccctctgtcgcccaggctggattggagtagcgtggtgcgatctcgtcccactgcaacctccgcctccggcttcaagcaattctcctgcctcagcctcccgagtggctggaattacaggcgcccgccaccgtgcccggctaattttttgttttgtttttgttttttaagtagagacagggtttcaccatgctggccaggctggtctcgaactcctgacctcgtgatccgcccgccttgacctcccaaagtgctgcgattacagcagtgagcccggccttttttgttgttgttgtttaaatgcatgaattgtttcctactaagaagctatgatatagttccttgaccaaatgcagatgaacaggattatctgataaattgattaacgagagcagacaaaatacgggtatttaattcatctgctcattnnnnnnnnnnnnnnnnnnnnnnnnnnnnnnnnnnnnnnnnnnnnnnnnnnnnnnngttgggaggggatacccagcttcctaagtaaaggagtgatatttgagcaaggcattatagaataaatgagagtttggccagtagggaaaagaagcagcatgcggagaggtatgggacgtgggggaatgacaagaacccagaattgaagtcactagaagatggtggtgggaggacgtgaaacaaggcaaggaaaggtacaagcggatccagatactggaagactttgtgttgcaatgctttaatgaaaaattggatttcgggccgggttcggtggctcatgcctgtaatcccagcactttgggaggctgaggcagacagatcacttgaagtcggggtttaagaccagcctggccaacatggtgaaaccccatctctacaaaaaatacaaaaggtagccgggcttggtggcctgtgcctgtagttccagctacttgggaggctgaggcacaagaattgcctgaacctggaaagtggaggtagcagtgagtggagatcatgctgctgcactccagcctgggcatagagcgagaccctnnnnnnnnnnnnnnnnnnnnnnnnnggttttattattaggctaagccataatttctttctactttttaaaatacagaaattgccatcaaactgaaatttattgtcttatatgctaataggtcagttgttctgtctttactgtaatgatttctttatgaaaatgatcttaaatctgagattcctaactttggcttttaactagaattacccattacatttgatgatgtctttaaatgtcagttgtagctattagtctgataatatgtgtggaatatacagaagggtatttagacagaagttaggttaacatctgaactacttcctccttgcgtatttaagagaatattgagttaggtttctagaatcctcaactaactctaagtttattttctttgtctagaatactatgctgtttttgtttttggaaggaagagatataagaacagtttgctgctctcaaggagcttcaaaggctgtaccagtggggatgccattggtatatttagctggatagttgttattcagaaaagcaggacaagtaattttgattcctggtccgtacttggtaatgtcagtaatgttaactctagctggttgttgacatctggtcatttagttgccaattcctttttttttttnnnnnnnnnnnnnnnnnnnnnnnnnnnnnnnnnnnnnnnnnntattctgagctcaattcaacacagggacaccaggttgctgcttttgttcatggtttccagcctgtgcacttaagaaatttatttttatgtatttcaagctgtaactccagagattgggattgtttgattgcgtccttagcagtgatactaatagcaacttctgtctctagaacattggaaaattaaaatgtatttatctaccgttttttcccttgaggttatatgaaggtagaaatgaatcagactagatgattagctaagcaaaactgttaaccctcatcccttcccctctagacaactatgaaattagtcagtatgtattcgatccttcttgcaatctcttctctgacaattataaaagtgattcaggctgcataatgttgtttgaatgaaatgaaaatacagactagagctgtttttggttttgtttttttttttatttaccatcagtctgttcagtgaaaactaacatttaagcatgattctttttaaaaatcattttgtgacagtttagcagggcttgnntgataagcaaactatggtatggtaatatttctagtgtgcacgtttcttcacatgtctggtatatgggaactctaactccatcaggactttgcctatagtaggtactcagcatttactgaattaaatcaataaacatttttgatgaattaaagtacaagtcagacctctgtgtctgtgggctctgcatctgcaaattcagccaactgtggatcaaaaatattagaaaaatggaatgacggtccaacaatacaagtaatacgaatgaaaacaatacaactatgtacattgtatcaggtattataagtaatttagagatgctttaagtataccaaaggatttgcataggttgtatgcagatactgtaccattttgtgtaaggaacttgagcatctgtggattttggtatttgcatggttcctggaaccaatccctcagggacactgagggactatagttggtcataccacctgattttagagattttctgatcctcagaagttaattaagtaaactacagtagtctgttcttaacctcggaggatacattccaagaacctcagtgaatatctgaaaccacatatagtattgaatccgatatatacacggtaatattttttcctatacatgtgtatctataaagtttaaattctaaattagacatagtattaacaataataataaattagaaaaagactgggcatagtggttcacgcctataatcttaacactttaacttttaactatgacgttgtcttgaaaaagaaatcagccagccaaggtggctcatgtctgtaatcctagtgttttgagaagctcagtcaggaagtttgtttgaacccaggaatccgagaccaccctaggcaacatggtgaaaccttgtttctataaaaaaaaaccaaaaaattagccagacctggtggtgcatgcctgtagtaccagctacccaggaggctgaggtgggaggagtgcttgaacttgggaggtcaagtttacaatgttgacaatgttgggtcctttacgtagttgtgtaagtgagccatgatcatgccactgcactacagccttgggcaacagcctgaccctgtctcaaaattttaatttaattttaaaaatgaaatagaacaattacaacaatacgctgtattactgacaagaagggcaaatttttaaaaaaccaatatgctgtaataagttatatgaataaggggatcctcccctaccccagaatatctgattgtactataccgtaggtaactgcaaccgtggagagcaaaaactgaagatactgtgtcttaagtttctttttcaactcccaaattcttggatttctcacgtcttggcttcctcagtagaggtgagaaatgctaaaacagtgaaaacaggaaaaataacttactcattcaagaagtcgattatggtccagatggaaaatttgaattatttttgtaaaactaaactaaagtagccaggcaccgtggctcacgcctgtaatctcagcactttgggaggctgaggcgggtggatcacttgaggtcaggaattcaagaccagcctggccaacaaggtgaaaccctgtctctactaaaaatacaaaaattagtcaggcatggtggcgggcacctgtagtcccggctacttgggaggctgaggtgggagaatcgcttgaacctggggtgtggacattgcagtgagcccagatcacgccactgcactccagcctgggcaacattgtgagactacaaaaaatataatagtaagtaaagtaaaaagtttcccatacttgataaatgtctaataaaaattgaatatgttctaggactctgaaaaaggagttgaatatagttggaggttggtttttaggaattatttttcttaaattaattatccttgtagtcacctaggaattgtatattttctgttgatcttagaaaattgatcaaatctatagttcattttgttttttcaatttttttttaaagagatggggtcttactgtattaatgttgaactcctggcctcaaccaggtctcccacctcagcttctgaagtaactgggattgcaggtgccactgagccaggctcctttattggtatttttattaaaagcttttctctaatgtctttgtaacagttctcaatttttgaaatggtgttactcattctttagagtaaactgtcaactttcattttcttttttttttttttnttttttctttctttnnnttgagacggaatctcgctctgtcacccaggctggagtgtagtggtgggatctcggctcactgtgannnnnnnnnnnnnnnnnnnnnnnnntctcctgcctcagcctccagagtagctggaattaacgggcacgtgacaccgtgcccagctactttttttttttttnnnnnnnnnnnnnnnnnnngatgggggtttcaccatgttggtcaggctggtctcgaactccagacgtcaggtgatccacccacctcagcctcccaaagtgctggtattacaggcatgagcaacagcgcccagcctcaactttcattttcatttggttagtttttgaactattcagtgggtaaggttgtataaatgtcttttctctgtatagaagtttcttggagttcaaggagtgctactttgcaaactcatagagtatttataaaagctaactgcagaaggtgttcataggctaaaccgtttcctattcttggtagcaccattttctctggcctgaaatactttccctctactattagtgtctgtcagtgcccagcagtgtatttactttcctgaggaacaattcaaatgctaagtgctttaagacctaagggtggaaaagcagtgttttcaggcattattaggaaaataagattttaattagacacccagaaacaaaaacaggtttgtaattggtaaagtgaaagatggttaaagaaggttagattgaccaaagctagagtttttctttttttctttttttttttttttttttttgagacagtgtctcnnnnnnnnnnnnnnnnnnnnnnnnnnnnnnnnnnnnnnggctagctgcaacctccacttcctgggttcaaacagttctcccacctgagcctcctgagtagctgcgaggcatgtgtcaccatgctcagccattttttgtgtgtttttagtagagactgagtttctccatgttggtcaggctggtctcgaactcttgagctcaggnnnnnnnnnnnnnnnnnnnnnnnnnnnnnnnnnnnnnnnnnnnnnnnnnnnnnnnnnnnnnnnnnnnntgtacctttttttaatcgagcaatctagttcttgatcctaatagtctttgtggtgggtgtttgcatttttagatgaggaaaagggaaatctaggagtcctaggaaatacagttggtatatgggaactgatacgtaattagacttaagcaatccatgttgaatttgtgacttaagcacttaactataaatttaccctctccagttgtctgatgataatcaaaacttgaagcagttatccatattgggatctctttggggaatcccagtcaccaaaagttaggttttctttaatattttttcatggaagttttcaaatatactcaaaattgaaagaattatgtaataaattctcatgagcccatcacacatcttcaataatgaatgtacagcattgcagtgtggagcttggcctattgctgaccactcatcaatgtggcagaaccactccatggttccccatggaaatgggagctacttcagttctctttttacagaaaaattcaataaatatctactgattgtgccctacttgtgacttgaagccaggtttgtttgttgttgtttttctttttttnttttttttttttttttactagtcttgctctgtcacccgagctacagtgcaatggcatgatatcagtgcactgcaacctccagctcctgggttcaagtgattctcttgcctcagcctcccgagtagctgggactatgggcgtgcaccacaatacctagctaatttttgtatatagtagagatagagcttcatcatgttgcccaggctgctctcgaactcctgagctcaagcagtctacccacctccacctcccaaagtactaagattacaggcatgagccaccatgccagcaaagctgggtatttcttaaatttgttcagtcaggtgcaagaaattatttgccctactctgaaagttaaaaatattctaagagaattatggtttcgcagctggggttttttaagacttgagctcctggggctcctgcatatatccttagaacaacattgtccagtagaagtacaatgtaagccatgttttgtttaacccagcatatccaaaatattaccccttttgcatgtgcttaatataaaaaattaagatgatttctattccaggttttcaatattcagtgagtaattttacacttagagcaagtgtcatttcagactagtcacattttgagtactcagtaaccacatatgggctagtgctaccttgctagatagcatagcctttgggtcccacaaagtgttcacattctctgtttacacacatcttttgaagtgtcatgagagagagccagataggatccagattttctttatatctgtttggtctgtcttacggattcctagttgattatttctttgttgcttcttatagggatgtatcagaagcttaataaactcatgattattatgttaactgcctgaagtattaattttagcaacaaattgaactataattttaatttttcaagtaaattttcttatgtgatactaagtatttcatatatacatgtattagaagtaaatgggagtttagtaatgaatagtatatagtagtttgtgattatatttttctgttttttttttttagattgcagcaaaaattggaggtgatgctgggacatcactgaattcaaatgactatggttacgggggacaaaaaagacctttagaagatggaggtaagttatactcatacgtattttaaattgtttttcagagtgtttagttgaagtggttctcagtattttttgttattctattgagacattacttttattataagattgttaaaactgtaaggtgtatattggcctaaagggggggaagagacctagagaaggtaagtaaaatttcagtcaaccagaaagttttgtcttactagaaaacagtttattttccctcttttaggattgtgccagattgaaagtttgcacagacgtcttgttaataatattaaaaaaacaaaaatataaattgcttagaagacacttcactggttttactcatacatgaatggattttaatatgctgtgttttcgtcatttttctatttgcaattacactttaaaggtcgaaattcgtattgtttgctactctagtgtgttgcaggttataccgttttttttaatgttctttgcttttagtacttttgtgtttcacgtttagctgtaaacctatagattaaaacagtgggttcacagtgctttgtagttttaaaaatcataatcattgaagcttctgaacttagaagtctgcatgtattttttgttttaggtttgatgtatgagttttgatacattttctttttacccttttttttaaaggaaggattctcactgaggctatagaatgtatttgtagcttttgaccaggagaactttgtttcctttatttaagtgtctttcatatttatagtgaggtttttaatataaaaaaaaaaaatgagctaatgatgcttcagatgttgtatgtaatgtattctttttattttatgtgtagatggctcttggacaagtccgagcagtacaacacactgggagggaatgccctctccttttaaaggcaggaatttttatttattacctgtgttcagtatgtaaacgtgaaataaaccagtggattcttaaatggacacaaatatttcttggattatgtgtctgaatttttgctgcacaacattctgatggttaatcatttaagtttgaaggggggaggagaatgtagtactttgagctataggttgtctgttccaaggtatgcattgtattcatctgtgtaatggatttagatgaaggtagtcatgtagttgccttgagtttttttgttttttgtgttttttttgctaaagttttatacagtgaaatgtttgtttataaataatagaacagtttaagttgagattgccttgtaatgttgtggggtttgtttttttttttaattttttggggcatagctttgtggtcactgtccgatatactcaacttttaatatgtcagatttttgtagtttgatacgcttttttccccccagtcttcagttcctgaggtggaagcatcattagcctttagcatgtgatattttgctagtaatggacctaaagtactttgtcttgtatcattctaatgtgcataacatacattaaggtcagtgatttgttaagaatcagataactattttaatgtctgtgcatcttttgaatgtgaagagaatgaagtatagtttcttttttagattactgagtttgtgttgaattttggcagttttggttcaaatgataaaccgtaccttcagatatttcaataaatgtttatatttattcttgtttgggaggggagaagctttgtacttaattggtaaaaaattaaaagacacttaattttgcagatcaaccagatgctaagaaagttgctcctcaaaatgactgtaagtattccttttaaactgggtcaaaagctatattaagattttgttcatattattgggtatctttgaagttagttggtttatgagtattttggatcagctagcctgaacttctttgtaaatatgtacctttttctcctattttacaatctgtcccattaattgtggccaggtatatgtaaggattggttgccgaattattttacatatttaacaactccaattcttgatctacgcttgtacaacttgaaaaaggaaattatattgttctgtgccattgctaatataatgtcttccctttcattggctctcttacccctgtcagtgccaatataaatattctgtataaaatttgactctcttcaaggtgttgtctgtgcattagggaaaggtttttgaatgtttgatgtgattttgttgttaaatacataagtaactttaaattttgaattttcgttttatttttatttaattttttgatagcttttggaacacagttaccaccgatgcatcagcagcaaaggtatagtcacaaacttttcaaaaagtactctgcaagttttggttgagctgcatgtaaaaacacaaccacattggtatatattgaatatgtgtctgtattttttggtgtacctagttcatatcactccccttgggaaggtaccataaagtgatgatttttcttttgagtgagaaaaatttgtgatttggagagagagagggaattagccacagtttagaagaacaggggtgaattagagtaactgttaagatgacattctctnnnnnnnnnnnnnnnnnnnnnacagttcatgccttcagactgttccattgatcatcccttcttcacttgatgtatcatctnnnnnnnnnnnnnnnnnnnnnnnnnnnnnnnnnnnnnnattttttgacatgagtctgagcatagaaccttagtttaagccattgggagacattagacttccatttttattaatagattatcttttatttgtaaacaaagtatctttcactgaaggaaaaatggtactttctgttactttttagcagatctgtaatgacagaagaatacaaagttccagatggaatggttggatttagtaagtaacttgatttttaaagttttgaaaacatgatcaaaacatactttagaatctttcaaccaaaaaaactttttttttctaactagtaattggcagaggaggtgaacagatctcacgcatacaacaggaatctggatgcaaaatacagatagctcctggtaatgttacattctcttggtattttcagagtgactagaaaagtagctttttttttttcttttaggtttctagtactaataatgctgcctttaatcttttgacctgaagttctatttttgttttaaattgtagacagtggtggccttccagaaaggtcctgtatgttaactggaacacctgaatctgtccagtaagtttgaaaatctttaaaatggacttaaagtaacaacgggagaactctttgaattttctctctgctctttgttactgctttattttacactactctttcgttgcctccttccctcccaagtcctctgactcctttgaagtttatgcctcatgcctttctcaattagggtttatcattaaacacagaaaatggttaaaacaacttcatatctactccagtctctacttacaaagcgaagtgtagcctggaggagaatgcgcagtaatgttgactggtgacacctaaaactcagacattaagctcaagtggactgttgtgttgtctgcatttccctagttccattcacttttccattcctctccaaggctctttaatactatatttccccatctccaaatcttcagcatctaaccccacctctctccttcttaagcttatttacggagaaaatggaaacgaatgataagaagcttttcttttcccctaccgctaaacctaccagccttcattttttttcnctgttcacatagtactcaggtaattgctttcctttgtgttcatgtgcctaaagccagccctttcttcctattgaaggttcagcctgcagttgtactttcttctgcgttattagttccccccctttactagatttttnnnnnnnnnnnnnnnnnnnnnnnnnnnnnnnnnnnnnnnnnnnntgcaggtgcatgccaccacacccagctaatttttgtatttttttagtaganncagggtttcaccatgttggccaggatggtgtcaatgtcttgacctcgttatccacccgcctcggcctccccaagtcctgggattataggcgtgagccactgcccccagcctgattttctttgtagcactttccaaataatacgttattcatttgacatgttatttttacttattttaatgaaacgaagccactccagcaggaaccctgtctctttgagtgttaccaccccattacctaaaatggcacttgcacatggttgatatttaagtatttgttgaatgaataattgtagcatatgagtaagtaaaatggtagtttaaaaatgtaaataaataaatcttttagttcttggaagaatcagtttaattctgagataactttagcattagagttcttggaaattgtggactattcttaaaaataaaaattgtatatctagaaaatttattgcctaatctctcaatctttgacccttgatggcattttctttcagttaaaagtaaaaacattgttaaagttagcattaaggcacctaatcctgaattggggtaggaggagtacttggttacattgttttgtatttctctatttgaataaacttgggtatgctgcaacttactatttaaatattaatttgttaacaggtcagcaaaacggttactggaccagattgttgaaaaaggaagaccagctcctggcttccatcatggcgatggaccgggaaatgcagttcaagaaatcatgattcctgctagcaaggcaggattagtcattggaaaggggggagaaactattaaacagcttcaggtattgttatgtttgtgaaatggctacttttggtctgttttgatgnnnnnnnnngtctgctcccttttgttaatatgtattattttctatgattataacaggaacgggcgggagttaaaatggttatgattcaagacgggccgcaaaacactggtgctgacaaacctcttaggattacaggagacccatataaagttcaagtaaacttaactttatactttataaagaaagagtttgggttgaatggggttgggcaaaatatgcatgaataattaaaatgttttgagacgtgctttctaaattagctaactttttctactttagcaagccaaggaaatggtgttagagttaattcgtgatcaaggtggtttcagagaagttcggaatgagtatgggtcaagaataggaggaaatgaagggatagatgtaagtaaaaatacccattcagaaatggttgtatgctaattcgtaaatatagtagtgttttctgttttgtgttaaatagctctaacattgttatccttttatttcacctttatactttagaatacagaattaagtattttatatcttgtcaccctatttgctataaatataaaattatatgtactattatgatttgaggcagattttcaggaaatggtnnnnnnnnnnnnnnncttttttttactttaaaccctgagaagctagttttcttaatactcagtcttttttacataaggtccccattccaagatttgctgttggcattgtaataggaagaaacggagagatgatcaaaaaaatacaaaatgatgctggtgttcgaattcagtttaagccaggtgagtgcatataataatcttgtaagtgttggcagcaatgagttttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacacccatttactgtttaattaattttgtttctttgttttgaagatgatgggacaacacctgagaggatagcacaaataacaggacctccagaccgatgtcaacatgctgcagaaattattacagaccttcttcgaagtgttcaggtttgatagaaagttaacattttcattttttgtttttatggaaaaatactttccttcatgaaatctgaagtttcctctatatcagagtctgcttgatgatgctttattaatggagaaagtttaaattgttttaaggtaaagatcttggagcaggaagaactactaccttaagtgctaccttatttactctttaatttaaaaaaatgttattacttatgattatttgggccggttcatctctacatttatggttcaggtatttgagagctgggaaaaatagacataattaattttatcatgaaaaaaagtatttctgtgctgattttttccttcttgtctattcctctctataggctggtaatcctggtggacctggacctggtggtcgaggaagaggtagaggtcaaggcaactggaacatgggaccacctggtggactacaggaatttaattttattgtgccaactgggaaaactggattaataataggaaaaggcaatgtattttaaactcttaatgtttaacacattattcatttttctggacactatttctgttgctgttgtaaacaagtggcaatgctttttctctggctgtgttttagtagaaaagcattcttatgttaatacgtaacaagtaaaacataaatgaaggatctaatgtatatttataaaaagagcaggattttaatcttactagcttctagagaaagtgaactaagataattattagcataagaaaggtcttttgacccaaaaagttgtttgagtgtttttgtttgtgcattttggtttttcccgactcatattttaaaaatttgaatgtttataagtgtattagtttatatttatctgcttttaaaagcagtttattcaaatattttattataatcacattaaggttaagtttaaacataccaagtaaatgtaaatgtattttaagagaagcatgaaatgcttcctaaaatttagatttaaagtgtagaatattaaatgaaaaatcttaatacaatactgtcaagtagaatactgactgaccaaaccatgtttttaatggcctatttaattgtgaccattttcttctaaatagcttctagtatacccttgaaacctttagagaaattactgtcttttattttaggaggtgaaaccataaaaagcataagccaacagtctggtgcaagaatagagcttcagagaaatcctccaccaaatgcagatcctaatatgaagttatttacaattcgtggcactccacaacagatagattatgctcggcaactcatagaagaaaagattggtgtgagtatactttaaacttttaatttatagtgtagacccttagattgtagttaaattaagacgtttattcgaatacannnnnnnnnnnnnnnnnnnnnnnnnnnnnnnnttatagatttcatgatacctataatagatacaatgtgaagattttccagcaatgaaaataacctaattaaatgtgcagttacaggttttgagaacaaccttacgtttgagtgtggatagataggagggtgcaggcatcatattagtgttattgtaggattgtggaacatacttgaaggacacagttaatgggaattcattatttattaagattttactatactgaaccccagcaaggcaaacaagataaatcagatgcatcttccgctctgcagtagaaattcgtaaatcctagcttttggactggctcacaaatcatctgggttttaaaatgtagactatttgggtgcttccaactcctacacataatatacagagacagacctgactcaaaatgtctgggatagggcccagcatctgattttacacagatgtttgggtttttttcttccagaaaagttttctgttagaacaaaaagtataaaaagctttgactgcttttttgtaaatgaggcagatggtgtcttactggagtttttaacacaaagtgtgcagggagcatcttaaattattaatcagaatttcctaggaaaaattagttttggtgtttgccatgctgtgaatggagtgcatgcaaaacaaggttgacgctgttttctgtgtacttgggaagacaaaaatagatgtactaaagatgcttaaggaacatcttataaaatgtacacaaacattgtaagttctgttgtatggatagtttaattgttcaaaaaatgaagatgtttgtggagtaatgataggaggttgatgccatagcaaaaaaaaatgaatagcacatcttggtttttgattttacagtattagccttacatgcatggatcaggatttctgttggattcatggtaaaacaagatagattgttttagagaagcaatttggtgtggtgattaagagcacggactcttttctctgaatcatttactgggtctgtgaccttggagaagttgctcaggcttttctgtgccttggtttcctcctataaaatgaggataattgtatctatttcatagagttgtagggattaaatgaacgttcacctgtgtgtcacttaaaagaatgcctggcacatagccctaaaaaatgttgctactttttcagtattatttttactatttggaaagaatagataatggataataaagtgagagatgacagcaattggagacttaaagagacaagttaataaaaataactttaaaggtcatgaagtttagtttccctattttgcagtgagaaatttacaacaaaatatagtgttaccattttgtccagcatgtcgcctggttgtgttaactactcagaaggagcattttaggacagttaagtgtaatgtctttgttggcttaacaaaacagaaatcagttaagcattattaatnnngatgtggcatgcatgcatgataaggatataaaatatcctgatttattgaaggacttaaaaagggaatgtttgtgctgtttagaattatgatacatgaaaggccaaaaaggatataaattattgatctgaatggggtttcagtgacagaaataggttgtgaggtggagtttggtttaatagtgaaccaactagctgttaacattatcaatgaagtgttcagaagacaattgatgatataggacccaagcagtttgggaatatattgtcaagatggttgtcttatgttaggcaagtagatgtagagagaagagctgaagataaagacctgatttctgtgattaagatgaaaaaagatagtaaaagaaagaattataaaaggaaagggatctactgctgtcgcagacaagctaaagaaaatttaattgctaattttaatttaatttaaattgcatttaattgctaccgctattgattttagtaaatttcacatgtctccttattatggcagataaaatagtttttgcaaaatgaatgaggaaaggaaggaaaacctaaaatttgttgtttgtgactataagagggtagaaatggaagattatttgtccatagagttggtttgctgatttttctatgtcttcctttttttttttgtagggcccagtaaatcctttagggccacctgtaccccatgggccccatggcgtcccaggcccccatggacctcctgggcctccagggcctggaactccaatgggaccatataaccctgcaccttataatcctggaccaccaggcccagctcctcagtaagtattgggtttagttctgggctttcccccaaagattctagtttttggactacatttttatactgaattttcttctcagtggtcctccagccccatacgctccccagggatggggaaatgcatatccacactggcagcagcaggctcctcctgatccaggtaaaaagatgcttatcatttgtgtgttagctgtattgtttttcactcgtgttacattattaaattttctagtgttgattctacatttgtatgcctcaccttcactcactctactctttcaacagcgttaggcactgcctctaccccagtgtatatagaactgacatgaatatgatctctgcttttatgaaattcctttcagcttgcatttggctttcttatgagtcgatatagcagaatgataaaaactaaaagctgcannnnnnnnnnnnnnnnnnnnnnnnnnnnnnnnnnnnnnnnnnnnnnnnnnnnnnnnnnnaatatgtagtgtgttaattgtgacattatctatgaaaaaatagtttttacnnnnnnnnnnnnnnnnnnnnnnnnnnngggcactgaggctcacgtctgtaatcccacctgttcagaagtcgaggctgaggcttgagaccagcctaggcaacatggcaagacccatctctaaaaaaatttgttttaaattagccagccagggtggtatgtgtctgtatttctagctgctcagaaggctggagggggggcggggattgcttgaggctagaagttctagattgtattgagctgtgggcgcaccactgcactcccctgggcaacagaatgggaccccatctcttaaaaaatatatatatatgtatatataaaacagtcttacagagtgttaagtatttgggcattaaactcccaaattgtcaaataagataccggttctagtactcctcataatgacaacttcatgtgagtaaaaatcaggcctgtatttaataactgcatgctaaaacccaaatacatttaattattttctatattcagatctgtattttcgatatgtatctattatatcctgtactattctggcgtctttccataccctgtagtttgctttccatcttggtcaaagagtcattctttgaaaccagttaacatttatttatgatctttttttttcacctgctacctaccttttaggcctctttcaccagagtagtttcaaggaaaatatactcaattatggattactttctacacataatnnnnnnnnnnnnnnnnnnnnnnnnnnnnnnnnnnnnnnnnnnnnnnnnnnnnnnnnnnnnnnnnnnnnntgctgtttggaatatgatgacacagcatgatagttaccaaataatcacaacttccattaattcaaggaatgacttgagttgtgctaggatataggtatgcaaagttggggttgtattctatagcaaaagaatgcactcccagcataccaacactgatagaagggctcacaaggtacagaatataatactgtaaacacgaatttttggagaatgaaagggctttgcttttcctcttgttggctaattgggatggtataattaagtaatgagattgaagagtttgagtaggttaagagacagattcatactgggtaacttggatctgccaggattgtatttttgagcactactgggtggttagcatgattgagaaaaaatgatgggnnnnnnnnnnnnaagtggtctttgtatcacatgttgagtttcccacttggagtagtagtgaagtcactactataaaagctggtcagtgaatgtggttgcagcatggcctttgggcaagaagtaacccatttaaanccagctggttggccccactcagatttatcaaagggttactgggtctctggggtggatattgcttatattagacttagaatgacgtaacgttttaatgtatgaactaaaatatttctttaaaaaaagagtggtctgttacggatttatgtagtggtcaagaatttagacttcagagtcaaatagacctagatcagtcctagtcctacagtttactaatggtgagatgtcaggcaagtttttgaactcctctaagcctgttttcttatctataaattgatcaatgaatgaatcttgggttgagtgaatatttagtaaattcttagtacatactagttatttgtgagactggtacttcagtatggtttacacgtttgggtgtagaaataacacttcctaaagtctgttttatctcaaattctctgtccaggcatagtgtaaagtgaaatacctagatttcttgattaatatacagataatagccgggcgccatggctaacacctgtaatcctagtactttgggagactgaggcgggcggttcacttgaggtcaggagttggagaccagcctagccaacatggcgaaaacctgtctctactaaaaatacaaaaattagctgggtgtggtggcgggtgcctgtaatgccagctacttgggaggcnnnnnnnggagaattgcttgagaatcgctccactgcattccagcctgggcaacagagtgagacacttcatctcaaaaaataataataaataccgataatgacactattgagatatgtaaacatccaggatacaaaagcagtacattgggcaattgagaaaagcttggagggtgtcctaaaaacaccttacaattatattaccttgtagttttctttttctacaaattctcactcctttttctcaaacttgactaacctttgttaagcagctgagaattgctactgttcagaatgaaagcataatagaaaatttaaaagttttaattgtatgatattcctagtataaaggacagaatcaagtttttttgtggtttctagaagattgagaggtctcaaactgtttgcacttcagttgatgtgggagatgagtgagggtcagtcaagtgtagaggaaacatagctaaaagctgagacatgggcatagtgatttctgaaaagtacaagcactgtgttgtggctggagcttgggtgttaaagagatacaaatgaagggagaggtgaggctgaaatggaaaggataagccaggggattcagactgttaaagatgttgaatgataggctgtacttaagctttgaccatcctgaagattctagagaatgaagaatttcaagtaggatgacatcaagtttataacattcaatgtgaaataggatgaaatgtggctaatcttttttttaagatttttatattttctcttcattgaaaataaggacaaagttcattgttctaaaataattgtttctttcttatgcaggttagcccctagaaatgtttttcttaagtcatcttccagatagagctgtttgtgcttgaggcgaaaccaatttagaaaaaaacaagggcacaggatggtttgagacagagcattggatttggagccaaaagacctttattcatatcccagttatgcaactcagtagtttttaacctgggaaagtcacttggtctctgagccttgttttctttaataaaagctgatggtaacaacaaaaataatagaaaatataaaanntttcatgtacttttttttaatgttctagttaattttgggagctgtatatttgccagagagctgggggggnnnnnnnnnnnnnnnnnnnnnnnnnnnnngtgagaattacagacttcaacatgcaaaccttgaactttcatttattctagctaaggcaggaacggatccaaattcagcagcttgggctgcttattatgctcactattatcaacagcaagcacagccaccaccagcagcccctgcaggtgcaccaactacaactcaaactaatggacaaggtaactaaagaacttatatgtgaaagtcaaaacttgtgcttggaattatatatgaagtacatcactgtataatacctaaaatttctaacattatttaattataatttaagcagacttttccttttttaaattgttacctggaaatagtttcaaactttcataaaagttataagaataccaggagccacccatataaccagctgttaatgttttatcccttgtttactctaataccgtctatatcatacaagtgtatgggtgtgtatttattttaaatatccattataattagttctgaaggaaactaattatatccagtataattagttctgaaagaaatcatttaagactaaactatagacacaatgtcctgttgggtacttcttcaaaaatatataaccatcatacagccctccaaatcagtaagtcaacattaatatattaagctctttatatcattatggatttctgatttttgcagtgggttatattttttgaatatttttataattttgataatcagactatcctagatttggccagtcagggagtatattcagggtggtgcctatatccttttgaaatatctgtcgttattttaagcacttccatactgtctggcacagtgagattgttattttgtgttttatgggctccaacccttaagtcagctatttcttcaaggagctctgattccttttagtagagtatggtagttagaaacgaggcttgactatgcttgttgctactgaggtgtaattgcttctaacttctttcagcagacagaactaggaaatatatttacatacatgcatacctacatacacatgccaaaaaacacataaacattgatatgtgtatatantttttaaaactatgttcatattacgacattcatttcagcattttggggttttcaagccttttccttttctgtacttgtttacaaatggtgagaaacctggtatcctcagtgtatctccttattttacatacatttacttatatgttcgacataaccagtcttccaaacaggttggttttcttttctgtccaccacctctgtacccccagtaccttctatctttggcaccagtagggatgccaccaccacatagtaccttcctcctacccctcactgtcacattgcaggcccctgccagctcctgcacccaaggaaacagctaaaattgccttttaaaaactttaattctgtttttctgttttgttttgttttgttttgcttgatcaaggagatcagcagaatccagccccagctggacaggttgattataccaaggcttgggaagagtactacaagaaaatgggtatgttttatacatttcttgaaaatacatacttaattaaattgaaacacaaggtattctcttcagaaagagaataattgaataaaatcactggactcgtaaacataccaagacagttgcaaattatagtttttaaatttgtggttatatagcaaggaaatatttttctttctaattgcatttgtcaaccagttattaattgaaactagaaatttcctcactggcacatacagtattaacattactatacttttgacaatgacagttatatatattagtctgagtgacataaggttaaattttaatgtgtcaggtgaaaatggattgtgtgtgataccattatttttgctgcaagataagcaggtaagaagtaatctgtagtgagggaaagtaactaagtgatggaaccagaatctggcttccaagagggtctgagtcccaagcttgtctcccaaatttgtctctttagggaccatttggaacctggtactatacttctggacaaatcactatgtttcaactgcttttgctctttaaaaattattacataccacagctagatgtcacaaacgaaaactaaattggtaagcttggttatccttcactagcagaaaaagaatctataggtggtagttttgtcataagagatcggtctacttgggattctcagtgtaagtttcaatgttttctttgccaaagaatgtttctcattctccgcagaaagaaaaatttccagaagggtgatgattttaatcttctaggtgtaaaattatatatacttgatgataaagatgttctgcacaactggtttctttttaaagaaagaaaaaattgtttttcctcatagagtggcttcctaggagagtcatattccatctatttctgagactataacagattaatatatgtttttgtgacgtagcttaggcagatctacagtagctgaaattccgcaaaaagaaacttttaacttaaaaacagcatactctgattaaggttggttacataatatattttctgaacagggatcttttttagaatgaatagggatgctattaatcatgtcctgncgggcgcggtggctcacgcctgtaatcccagcactttgggaggccgaggcnnnnnnnnnnnnnnnnnnnnnnnnnnnnnnnnnnnnnnnnnnnnnnnnnnnnnnncgtctctactaaaaatacaaaaaattaaccgggcgcgatggcgggcgcctgtagtcccagctactcaggagcctgaggcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaaaaatcttcaacttctattttctcaatgggtaatgttccctagatatggtccctagttattannnnnttaagtagataaaaataaaggcttgttaatggatttagctaattactgaggaatgagtttgacttctgnnnnnnnttttaccttattgattatttgtaatatggccgttaatacatttactgtttagtcttttatgttttacttttttatgttttactcnntgagtgggtgggttgaattctgatttttattgttaagggaagaaattctaattttcattgttaagggaagaaattgtctttattgtctttaattttttttttcaccatttccctgccagttagagatactgtgctatactgtcttaaatcctctgtaggaaaacatggcatagaaataattaaataataatactgcatgactaaagaacctccttaccccaccttttctatcatttttctgtagtaccacaataatcacctgttaattttatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcacctgttaattttatgagcaaaatagattagttgaaaacagatgtaggttaagttttccaaaattggctttatattttaaagacaattctaaattccccaggataacaccaaactaagttatttttttttttntttttttttnnnnnnnnnnnnnnnnnnnnnnnnnnnnnnnnnnnnnnnnnnnnnnnnnnnnnnnnnnnnnnnnnnnnnnnnnnnnggttcaagcaattcttctgcctcagcctcccaagtagagaaaatcttgatgagatttcagagagttataaggaatggcagcaatctaaagatgggtatttttaaagctagaatagaaggattttttataagagactcttaagagaacaagcagggtttcaaagtagaaaggagtaattcttttttggagaagtgaagacatttgagaaaagtttgacttaaggaaggcccttacaataaggactgatgtagtagactctgaacccttaagaatctggtaccttaagcctaaaggagggtagagtgagacttgtaggaaaggtacaagtatataggaagacaactgagtgtgaaaatctattcaagcaaggctgactcttaggctataataaaatcttacaggctaactgtagagcaagtcttttatcttttggatgaaagaaatgtaaggctaaactttatagattataattatnaaactataataaatattagataatgttctactgaagtgtaaactcaactgtcttgaatcaaacagtaactaagccatgatcacaccactaccactgtgttccagcctaggtgacagagcaagaccctgtctcaaaacaaaatccaataactaaagagaataataaggggagcaaggtaaggcagtacatgtctgaaagggcaaggaaagagttctttaaagtctaaagtgtttactattagctttccacagggatataggagtttgcagacgttgtagttttttaagggaacattgatggaattttcttgatgaatatgcctgtttctaccatcttacatctaaaatgagtaactctcacagtgggtttaaaacctgaagaatattgattgctaccttgatcaggtttggtttcaagtgtgcagcttgtaagagaaaacgtgtggttttattgctgttgttacacgagagtatttggcagttttttaaatattgaaacctgtttcagatttgcttgaattgtgtcttgtaagaggcaaatgttcctgtttgattaacagttctttatagaagacaggaaaatatgagaatttttaatagtctatgagagttctctgttacccaagaaaagagggtttgtttcatgcatttttaaagaatcataaatcttaaattctttcactcagttgctttgattctgtcacctattttacaatggtgatggacaggtttctgaaactaggaaattggccttgttggcaacatcatacaaacataaaaacagcttcctatacgtccatgctccagtacttttgtctgtggtaattatttcacaaagccaagtgagttacaaacgaaataaaatagtgcttaagtaaagcaactgaataggaaaacacatctctcttctcaattttttatttagaaaattatataccttcagaaattggggaaattaggtaccactcagcttcattcatcatttttttaaagtttgctgtatgtgctttatatgtacgtttttctgtatgaaccatttcaatgtaagttgcagacaagacagttggctctaaagaaactcaatgtctggacacagtgattcacgcctgtaatcccaggacttttgggagaccaaggcaggtggatcactttagcccaggagttaaagaccagcctggacaacgctgcaaaaccccatctctgcaaaaaaagagaaaaatnncagctaggcatggtgggcttgtagtcccagctactctagaggcagaagtgggaggatcatctgagccgtgggaggtcgaggctgcagcgagccatgaatgcaccgctgcactccagcctgggtggcatagtgagaccctgtctgaaaataaataccttcaatagtatcacaaaaatacagactatatttaaatttccatagttgtcaggaatatgtctttcgtcgtctgtgtgtttgttaaattcagagtgcattcaaagattcgtgcattgcatttcattgttatatttctttagtctcttaattcagcaaagtcatctcatttggagaaagacctgatcgtgaaagctgagttatgtgtttcattgatcttgttctttattccttgtcacgttacttttaataccattgtgttgtaattggaataaattattgatacttttaacatcttactactaatctctgtgtgtgtgtacatgtacatgtacatacactttataggttactaatcaggaaaaagtcttggctaggtcttaaaataataaaatctttaaatcattgcaattaagtgggttttttgtttttaattctggaatccaatcaagattatgcctaaatctttaccttccttagctaaagcagtgtggatttggggttgattttgtttttttactaataatgacgctctagaactaaacattatgattaattataaggcaaaaagaaaataaagagcgttttttttttattttagagtagtttcctgacactactaatgaatattttaaataaaacaggagtaattctgaccctctgtgcttttgtcttatgattgtaatttaatattaaatttagggggtttttttaggtcaacaagggcagacacaagattattcaaaggcttgggaggaatattacaagaagcaaggtattgtttttactggaaatgaggtgttggacttttaattgtggttacagcaacaaaattcttttttttcaaaggtcaagcagttcctgctccgactggggctcctccaggtggtcagccagattatagtgcagcctgggctgagtattatagacaacaagcagcctattatgcccagacaagtccccagggaatgccacagcatcctccagcacctcaggtataatgtaattgctaatttgttgatttctactccagtctgttttctgcatgtttactgtttgtctgtttgggagtgtttgccttttaaatttttatctggcaaagtataacttatttaaatgaagtactacggtgtattgtttgggtttttttgtttgttttttataatgttttccggcatctgagtgctgaatatttctgcaatgcctttgattttaaaaataaattttcttcccccagggatttgcaaatcatgcaagaagctaccaccatttatattaaccagtttttctttcttaaaggattcactcctgaattagctccatttcaaggattttctttaactttttgtgtatttcttatgtatctcttctgcacagggccaataataagaagtggacaatacagtatttgcttcattgtgtgggggaaaaaaacctttgttaaatatatggatgcagacgacttgatgaagatcttaattttgtttttggtttaaaatagtgtttttttttgtttttttttgttttttttttttgcaaatgtacaaaatatctatcactactgataggaggttaatatttctgtgtagaaatgaaaattggtttgtttttagtatttagtgtagatgtacacattccagcaaatgtatttgcaattatgtggttgatgctttgtgatataaatgtactttttcaatgtatactttcacttttaaaatgcctgttttgtgctttacaataaatgatatgaaacctcctgtgtcggtaagttggatatgtgggtatttaaaggattcataatttcttagcaatgataaattaagatacatatacacaaatatataagctttccccatgaaatattgagtttttaaacactggcatgtttttccccccttgcagtatagtggtagattggaggatcttttccatttattgtatttggctctttcagcacaagtaatcctggtatcttcatttttttccttctgtttgattaaaaactgcatgtgtgtacaatgatcttctggcatacttccattgcattaacagtgaaatttcctttttatacatgaccactgtttcagacctgtactgctgctataacagttaacctttctgttcttaatttgataatacttgatttccaagactgtttcggcataactaattttaaacagttttcagatagtgaatatgagtagtctaataagaacagtttttttccatgtaaagcaactctttcaatgtatataatagtgtgtttctttctaaatttaggatagaaaagtgaatagtgtgcaaaaagtatagctacattgcatctgccattgaaacataaatggggtatggaaacgttcaagctttttttttttctttaagcagtatagataagctttgttttgtaaatgcacaagtccaatcattgaatcaacttaatttttttatgtacttgaagtcattttattactctttaacactcatgctgaagttctgatattttgttgaaatccattgttttactctttgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagaatgacaaattgatgaactgcaacagtatctttttgtcaatgttccacataatgcaaatgccatactttgtgtgaatattatgttggaatacagtgctgatatcttggaaaaccataactgcttcttaatttaacatagaataatacatagttctgtattttttttaaagtgagcttaatgggtaagtattttttatatgctttagctatagctaaagaaaactgatacttaacaaagttgaatagtattattcactggtgctcctgaaatattgtttttcagtgtaaaatatgcatatattctatatttaatatgaaagtcttgaaatgtatcagaagggatttcagtttgcaaataatgagcaatgtagcaattttaacacatttcataaatatatattttgtcatttgtggggagcaccatttgttgttttgaatatactttaaaggaagaggtacaaggacataaatgttgagattacctacaggatggaaatagcagtacagttcattatagatattttgaaatgtttttgattgttttatataacctagagtgacttcccttacccttatttagatctggatatatagttctagtttgaagtttaatagttaaggagttagctatttgttatctttaagagtagggtattgacgtgagcaattgcagtattttgcatgatactgttttatagatgaccttttaggaaagtggtgcatttattaattgaactgaagaagtagttcagttgaattcagtatcataactcacaaattggaggctgttgattttgattcatttaaggtttaaaatctttattaattgcaaacagtgcaattatttatacttcacagtgccttcccagaccttccaccttaggttctgctgcaaaaagcaccaggtaagcacaacctaaggacatatataaataaatatttcagtacattaatgttgtccctgtgaggtttttgtggttgtgtattcaaaggcaatctgctnnnnnnnnnnnnnnnnnnnnnnnnnnnnnnnnnnnnnnnnnnnnnnnnnnnnnnnnnnnnnnnnnnnnnnnnnnnccatcttagtggaaagtctgtaagttgttaaagcaactgtttacatttctgggtaatgtttttatttacgttttttttttttatttaagacaagaaaatgatgagtagattgctgcagtaattgatctacatccaaatctttttgtattttttccccaaatatagaagtgttaagattaagaaaggacaattacacagttttcaagatttaggaaatcacttgttcagaaacttcaacagccttcacaatctgttttatatgatggacagaaaatttctttgccctccaaaactataatttctttatttttttcttaaactataataattcagtaaggatattatgggttacaattttattatgttttttctgagacaaaagttatatgctagaaaaggaaaaagttaataaggcagtatgttttgataaaaggcatgtgcatcagtgaaatgttaactgtacagcaaataacctttcataatctgtagcaatcagtatttttctgatttaatattttaataactgacgctgcatttatataatttttttgccagtttaaaatgtttgtgtgtttttatagatgattttaactggtacatattttgagttaagatgaatgtatgaaagcagcatcttaccagttttgtttattcaatttctaaaatgtgctgatccttttaaaactcctgcttatctctgcaacaaagaaaaatattcaaaaatactgccttcattttcacacacagtgctgaagatgctgcaagcaccaaatcatagctcaataaaatcaggtcctgagatagttacccataaagaggaatcctttgagtgtatgccattggtgagccgatgagcatggaccatagaagggctcaatgtagaaggtaaaattggcaaatcataattgagaaatatgaaatgtattcccatacataatatggtatagggtgtaatgtacctgcttttgatcacttttcattttaaagtgctattcacttgatcttaaatgttccatgaactgttaaatttcttaagttacatagttattacaccacatttatgtgtatgttatgttttaatagtcaatgataggtatgtaaatataaagggactcattgaaacttgagagcctgtcgagttttggttagttgtagattgcatttttattaaaaaaatatagatagatgaatgataatagatattggggcactgtttctgtctcatgagaattcttttattcattaccataagccttcactgataatataagcattattttaaatgacgctggtcttaaatctgaaataaatggaaagcagaaaaggtgagccagttgatttgaatgcattggatattagtgttagaaacaatgtatagtttagattgaaattgaactgactttatttagcacttaaacaaaaatttgacaatgtttttggtttttttttaagacagcttagtgtggtgatacttagaattctatggtttgatgtttcttttagaaatgagaagtatagttttattttttaatatcaaaaatggttttaatactaaaactagtaatttaatactagttgtttataaacattgtaaaatatatcttttaaacaaattatcttagtagttaattcataagggtggttttgggtaggaatagcagagtaccttcagagggaaaggggagtaattcagaagtgatagcattttatttgtttgaatactctgccagtaaaatcagctctacttagaaagttatctgttgtgtagaataatgatgtagagtttactaatcagtgaggatgtcttgtttttattttctgcaaactctgcctcactttaaaatgcattttaacaatacctaattaaaaataattttggttctgaaaataaccttattttttgttgagttagtgacttcatttttcttgtcacaatataagcttttgagggatttttttaaattggtgcttttaataagcaaatcccagggttttattttcttcagtgatacccctcttaaatgtatttgcacatatatatatattttttcttatgcatgctcgatgcattttcgtcctgagaaaaatgttctctacagaaactacccgtgtgtaaaaagaagattggcttaaaatggctactgtgatgggaacagtgtcttagggagatgcagcttggacttgaggtaaattgaatactttacaaactgtggtttagagtttgctttaatgacattgtatgtaaaaggtcacatgatagctgtaattttgtattcattatggtttcctcaataaataaatgtacattgatgaatattataag

SEQ ID NO: 6 - DNA, 1968 bp, Macaca fascicularis
  source 1..1968: mol_type = genomic DNA
atggcagactattcaacagtgcctccaccgtcttctggctcagctggtggcggcggtggcggcggtggtggtggaggagttaacgacgctttcaaagatgcactacagagagcccggcagattgcagcaaaaattggaggtgatgctgggacatcactgaattcaaatgactatggttacgggggacaaaaaagacctttagaagatggagatcaaccagatgctaagaaagttgctcctcaaaatgactcttttggaacacagttaccaccgatgcatcagcagcaaagcagatctgtaatgacagaagaatacaaagttccagatggaatggttggatttataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattcctgctagcaaggcaggattagtcattggaaaggggggagaaactattaaacagcttcaggaacgggcgggagttaaaatggttatgattcaagacgggccgcaaaacactggtgctgacaaacctcttaggattacaggagacccatataaagttcaacaagccaaggaaatggtgttagagttaattcgtgatcaaggtggtttcagagaagttcggaatgagtatgggtcaagaataggaggaaatgaagggatagatgtccccattccaagatttgctgttggcattgtaataggaagaaacggagagatgatcaaaaaaatacaaaatgatgctggtgttcgaattcagtttaagccagatgatgggacaacacctgagaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccaacagtctggtgcaagaatagagcttcagagaaatcctccaccaaatgcagatcctaatatgaagttatttacaattcgtggcactccacaacagatagattatgctcggcaactcatagaagaaaagattggtggcccagtaaatcctttagggccacctgtaccccatgggccccatggcgtcccaggcccccatggacctcctgggcctccagggcctggaactccaatgggaccatataaccctgcaccttataatcctggaccaccaggcccagctcctcatggtcctccagccccatacgctccccagggatggggaaatgcatatccacactggcagcagcaggctcctcctgatccagctaaggcaggaacggatccaaattcagcagcttgggctgcttattatgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaagcagttcctgctccgactggggctcctccaggtggtcagccagattatagtgcagcctgggctgagtattatagacaacaagcagcctattatgcccagacaagtccccagggaatgccacagcatcctccagcacctcagggatttgcaaatcatgcaagaagctaccaccatttatattaa

SEQ ID NO: 7 - DNA, 6825 bp, Macaca fascicularis
  source 1..6825: mol_type = genomic DNA
ccttagccccacactagcgcgttgcccggaaacatcgcgagatttcgttccttcagtctccacccctttacggcacgatggtcgcgcaagaatgtgatacagcttcgacggccgccattttctttctttcttagctgttagctgagaggaagtctctgaacgggcggcagcggctagcgtagtgtaaccatggcagactattcaacagtgcctccaccgtcttctggctcagctggtggcggcggtggcggcggtggtggtggaggagttaacgacgctttcaaagatgcactacagagagcccggcagattgcagcaaaaattggaggtgatgctgggacatcactgaattcaaatgactatggttacgggggacaaaaaagacctttagaagatggagatcaaccagatgctaagaaagttgctcctcaaaatgactcttttggaacacagttaccaccgatgcatcagcagcaaagatctgtaatgacagaagaatacaaagttccagatggaatggttggatttataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattcctgctagcaaggcaggattagtcattggaaaggggggagaaactattaaacagcttcaggaacgggcgggagttaaaatggttatgattcaagacgggccgcaaaacactggtgctgacaaacctcttaggattacaggagacccatataaagttcaacaagccaaggaaatggtgttagagttaattcgtgatcaaggtggtttcagagaagttcggaatgagtatgggtcaagaataggaggaaatgaagggatagatgtccccattccaagatttgctgttggcattgtaataggaagaaacggagagatgatcaaaaaaatacaaaatgatgctggtgttcgaattcagtttaagccagatgatgggacaacacctgagaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccaacagtctggtgcaagaatagagcttcagagaaatcctccaccaaatgcagatcctaatatgaagttatttacaattcgtggcactccacaacagatagattatgctcggcaactcatagaagaaaagattggtggcccagtaaatcctttagggccacctgtaccccatgggccccatggcgtcccaggcccccatggacctcctgggcctccagggcctggaactccaatgggaccatataaccctgcaccttataatcctggaccaccaggcccagctcctcatggtcctccagccccatacgctccccagggatggggaaatgcatatccacactggcagcagcaggctcctcctgatccagctaaggcaggaacggatccaaattcagcagcttgggctgcttattatgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaagcagttcctgctccgactggggctcctccaggtggtcagccagattatagtgcagcctgggctgagtattatagacaacaagcagcctattatgcccagacaagtccccagggaatgccacagcatcctccagcacctcagggccaataataagaagtggacaatacagtatttgcttcattgtgtgggggaaaaaaacctttgttaaatatatggatgcagacgacttgatgaagatcttaattttgtttttggtttaaaatagtgttttttttttgttttttttttttgtttttttttttttgcaaatgtacaaaatatctatcactactgataggaggttaatatttctgtgtagaaatgaaaattggtttgtttttagtatttagtgtagatgtacacattccagcaaatgtatttgcaattatgtggttgatgctttgtgatataaatgtactttttcaatgtatactttcacttttaaaatgcctgttttgtgctttacaataaatgatatgaaacctcctgtgtcggtaagttggatatgtgggtatttaaaggattcataatttcttagcaatgataaattaagatacatatacacaaatatataagctttccccatgaaatattgagtttttaaacactggcatgtttttccccccttgcagtatagtggtagattggaggatcttttccatttattgtatttggctctttcagcacaagtaatcctggtatcttcattttttttccttctgtttgattaaaaactgcatgtgtgtacaatgatcttctggcatacttccattgcattaacagtgaaatttcctttttatacatgaccactgtttcagacctgtactgctgctataacagttaacctttctgttcttaatttgataatacttgatttccaagactgtttcggcataactaattttaaacagttttcagatagtgaatatgagtagtctaataagaacagttttttttccatgtaaagcaactctttcaatgtatataatagtgtgtttctttctaaatttaggatagaaaagtgaatagtgtgcaaaaagtatagctacattgcatctgccattgaaacataaatggggtatggaaacgttcaagctttttttttttctttaagcagtatagataagctttgttttgtaaatgcacaagtccaatcattgaatcaacttaatttttttatgtacttgaagtcattttattactctttaacactcatgctgaagttctgatattttgttgaaatccattgttttactctttgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagaatgacaaattgatgaactgcaacagtatctttttgtcaatgttccacataatgcaaatgccatactttgtgtgaatattatgttggaatacagtgctgatatcttggaaaaccataactgcttcttaatttaacatagaataatacatagttctgtattttttttaaagtgagcttaatgggtaagtattttttatatgctttagctatagctaaagaaaactgatacttaacaaagttgaatagtattattcactggtgctcctgaaatattgtttttcagtgtaaaatatgcatatattctatatttaatatgaaagtcttgaaatgtatcagaaggggatttcagtttgcaaataatgagcaatgtagcaattttaacacatttcataaatatatattttgtcatttgtggggagcaccatttgttgttttgaatatactttaaaggaagaggtacaaggacataaatgttgagattacctacaggatggaaatagcagtacagttcattatagatattttgaaatgtttttgattgttttatataacctagagtgacttcccttacccttatttagatctggatatatagttctagtttgaagtttaatagttaaggagttagctatttgttatctttaagagtagggtattgacgtgagcaattgcagtattttgcatgatactgttttatagatgaccttttaggaaagtggtgcatttattaattgaactgaagaagtagttcagttgaattcagtatcataactcacaaattggaggctgttgattttgattcatttaaggtttaaaatctttattaattgcaaacagtgcaattatttatacttcacagtgccttcccagaccttccaccttaggttctgctgcaaaaagcaccaggtaagcacaacctaaggacatatataaataaatatttcagtacattaatgttgtccctgtgaggtttttgtggttgtgtattcaaaggcaatctgctactgcttccccaaaatgtattttgttattttatgctaccatcttagtggaaagtctgtaagttgttaaagcaactgtttacatttctgggtaatgttttttattttacttttttttttttttattaagacaagaaaatgatgagtagattgctgcagtaattgatctacatccaaatctttttgtattttttccccaaatatagaagtgttaagattaagaaaggacaattacacagttttcaagatttaggaaatcacttgttcagaaacttcaacagccttcacaatctgttttatatgatggacagaaaatttctttgccctccaaaactataatttctttatttttttcttaaactataataattcagtaaggatattatgggttacaattttattatgtttttttctgagacaaaagttatatgctgaaaaaggaaaaagttataaggcagtatgttttgataaaaggcatgtgcatcagtgaaatgttaactgtacagcaaataacctttcataatctgtagcaatcagtatttttctgatttaataatattttaataactgacgctgcatttatataatttttttgccagtttaaaatgtttgtgtgtttttatagatgattttaactggtacatattttgagttaagatgaatgtatgaaagcagcatcttaccagttttgtttattcaatttctaaaatgtgctgatccttttaaaactcctgcttatctctgcaacaaagaaaaatattcaaaaatactgccttcattttcacacacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgaaatgtattcccatacataatatggtatagggtgtaatgtacctgcttttgatcacttttcattttaaagtgctattcacttgatcttaaatgttccatgaactgttaaatttcttaagttacatagttattacaccacatttatgtgtatgttatgttttaatagtcaatgataggtatgtaaatataaagggactcattgaaacttgagagcctgtcgagttttggttagttgtagattgcatttttattaaaaaaatatagatagatgaatgataatagatattggggcactgtttctgtctcatgagaattcttttattcattaccataagccttcactgataatataagcattattttaaatgacgctggtcttaaatctgaaataaatggaaagcagaaaaggtgagccagttgatttgaatgcattggatattagtgttagaaacaatgtatagtttagattgaaattgaactgactttatttagcacttaaacaaaaatttgacaatgtttttggttttttttttaagacagcttagtgtggtgatacttagaattctatggtttgatgtttcttttagaaatgagaagtatagttttattttttaatatcaaaaatggttttaatactaaaactagtaatttaatactagttgtttataaacattgtaaaatatatcttttaaacaaattatcttagtagttaattcataagggtggttttgggtaggaatagcagagtaccttcagagggaaaggggagtaattcagaagtgatagcattttatttgtttgaatactctgccagtaaaatcagctctacttagaaagttatctgttgtgtagaataatgatgtagagtttactaatcagtgaggatgtcttgtttttattttctgcaaactctgcctcactttaaaatgcattttaacaatacctaattaaaaataattttggttctgaaaataaccttattttttgttgagttagtgacttcatttttcttgtcacaatataagcttttgagggattttttaaaattggtgcttttaataagcaaatcccagggttttattttcttcagtgatacccctcttaaatgtatttgcacatatatatatattttttcttatgcatgctcgatgcattttcgtcctgagaaaaatgttctctacagaaactacccgtgtgtaaaaagaagattggcttaaaatggctactgtgatgggaacagtgtcttagggagatgcagcttggacttgaggtaaattgaatactttacaaactgtggtttagagtttgctttaatgacattgtatgtaaaaggtcacatgatagctgtaattttgtattcattatggtttcctcaataaataaatgtacattgatgaatatta

SEQ ID NO: 8 - DNA, 1959 bp, Macaca fascicularis
  source 1..1959: mol_type = genomic DNA
atggcagactattcaacagtgcctccaccgtcttctggctcagctggtggcggcggtggcggcggtggtggtggaggagttaacgacgctttcaaagatgcactacagagagcccggcagattgcagcaaaaattggaggtgatgctgggacatcactgaattcaaatgactatggttacgggggacaaaaaagacctttagaagatggagatggctcttggacaagtccgagcagtacaacacactgggagggaatgccctctccttttaaagatcaaccagatgctaagaaagttgctcctcaaaatgactgtaacagatctgtaatgacagaagaatacaaagttccagatggaatggttggatttataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattcctgctagcaaggcaggattagtcattggaaaggggggagaaactattaaacagcttcaggaacgggcgggagttaaaatggttatgattcaagacgggccgcaaaacactggtgctgacaaacctcttaggattacaggagacccatataaagttcaacaagccaaggaaatggtgttagagttaattcgtgatcaaggtggtttcagagaagttcggaatgagtatgggtcaagaataggaggaaatgaagggatagatgtccccattccaagatttgctgttggcattgtaataggaagaaacggagagatgatcaaaaaaatacaaaatgatgctggtgttcgaattcagtttaagccagatgatgggacaacacctgagaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccaacagtctggtgcaagaatagagcttcagagaaatcctccaccaaatgcagatcctaatatgaagttatttacaattcgtggcactccacaacagatagattatgctcggcaactcatagaagaaaagattggtggcccagtaaatcctttagggccacctgtaccccatgggccccatggcgtcccaggcccccatggacctcctgggcctccagggcctggaactccaatgggaccatataaccctgcaccttataatcctggaccaccaggcccagctcctcatggtcctccagccccatacgctccccagggatggggaaatgcatatccacactggcagcagcaggctcctcctgatccagctaaggcaggaacggatccaaattcagcagcttgggctgcttattatgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaagcagttcctgctccgactggggctcctccaggtggtcagccagattatagtgcagcctgggctgagtattatagacaacaagcagcctattatgcccagacaagtccccagggaatgccacagcatcctccagcacctcaggtataa

SEQ ID NO: 9 - DNA, 26405 bp, Mus musculus
  source 1..26405: mol_type = genomic DNA
gctgaaacatcgcgggatttccgtctgccagtctccgcccctttacggcacgatggtcgtgcaagaatgtgatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcaggtaagcgtggaccgaccgcgcggcggcttcccctccccgacccccgagagccgccggcggggcaggtccggccctgctcgcactcgcggctgccgcccgaggagatggcgtcttctcacgaggggtcccagcggctcgaggccgagcccgttgtcgtggtctgaagcgcccccccctcccacgcgctctccccgagcgtggcctccacttccgccccgcgcgcgcggcctcgccgccccgtgcgccctgcctgccccggctccctcggctccggttccgcccgacccaggcgcgtggaaggttctgggcgctcaggtcacggcgagggcaagccagggagggccggctgcctggctagcggtccccgccgcttcccgcgccgccgagctctccaccctcgccgtaattagcctaacgttccccgcggcgttcaccagcgaggctctccccggggcgggatctgcgtgcgatccaccaatcctttccccagctccaaatggccccgggccctttcttccccagccttgagtttgaatctgcctctctcttcccaagtcgaagcctccaacctctttgtgctacgtagggacgtactgttgcctcgaatggcaaccattttcccagatgaaaagggttggatagataagagtccttgcatttgacaccccccccccaataacctgaacgtcaatgtcttcaagtagcccccgcccccgacttctttgtagcttaaaaatgtataatgttgccttgccgggtggggtggtgctcgactttagtcccagcactcgagagacagagcctggcaggtctcttgagtttgaggccagtcaaaacggccaagagggacctgtctcaaaaaaaaaagtatgttatggaatttgcctgtaaggtaaaataaacccttatggctctgttgtagttgctagacatgagcaggtttttaaagccagctttgactatactggaaacttccttcaggaccgaaagattaccgactcaatcaaggtttatcaagtctaccataatacagcaagcacacttatctagtgcatagatactgaaaaaatatatttattcatacagagggattccatgagcaatgtggttggtgtacatagaagtcagaggacaacttgtgggagttgattctccttccatgtaggaccctttggtcaggtgtaagttgttaggctcagggacaagtgtgtctgcctgggcatctgaggaaactttttgatgcagggcctctaccgaaagcctaggcaggcctgaggtttaaggcttcctgctgaattgtctctagtgtaggtgtactccacctctcggctttggaagctttttaagtgtgtgatttctagtcttgtgagataaaaatcgaacacgagtgagcagacagctttgatgataaagtaaaccacagcggttacataaatagtcacactgtgaggcaggcgctgtacaggacgctgctggaaagggatgcctcactgaaagatgggtgacattcatatagtgcagtgaggagtgggagtctggccagcatagacaagaagctgctgcttgctctacaaagatcgagggtttctggaaggacagctctaagaggtagtcagctaatataggataacataggaggaggaaaaaggtgggaatgtgagaaaaactatggatagattccattgttagcaagcattggctgtgataatacaagtgtttattatagatgatagtatctgttacacctttggacattctgggtatgtagaaattccatcaggaccttgcctgtgtaagtaagggactaaggtctcactatgtagttctggctgtcctggaactcactgcctacctctgcctttaggactaaaggtgtgtgccacccatgcccagatgagattttgttttcaaaatgagagtctttctgcctcaccctcctgaatgctggagtgacaggtctgcctacactgatcctggcacacttactttttctaaaaattattttctttatttacattcctgcccactcacccccagttcctcacaccattccttcttcatctccccacctcccttccctggggcctcaagtctcaagggttaggtggatcttctcccactgagaccagaccaggcagtcctctgctgttacacacacacacacacacacacacacacacacacatatacacacataagccagggaacttggaccagcccgagtgtgctgctaggcacactttttataagtttggtgcaactgtgtctttgaggatgaatgtgcgcattgttaaattttccaaaaggaaaatttgaaagttgctgtggaccctgcctaggaagcactagaagtagatttgcacttccacctttaaaactactaaagaaagtgtgggctaaagtgtaggagatagatgttttcacgtcattaccttcttagaattatctttaactccaaaattggtgagggtgggtcagtgaagaaagtggtaaatgctggtttggtttgatttttgtctgaagtaaaaaagcacagaaatacagtagagcaaaaatcaagtggaaagcttgaattatttctgtgggttaaaataaaaacattacttacacttaatgtgtgtttaatagaaacattgggttcataattgtagcatgtgagaagttaggattagactagctggggctataatcagaccatgttcccatccacccaccacccacccccaatgaggggagagttgaaagttttgcttggtcttttgaatgaactgtgtaccaatttttcttttggatgcttgtagacctattacatgaatggagtgtgaataagcatgctttcttatggatattccttggtcttcaagtaaactcagaagtgttcataaaagctaattgtagaacatagccatagtctaagccgttttcctctcgttcaattgtacgtaacacttcccttttaacatcagttcctgtcagtcccatacagtgcacttaaatacctaagatatagtttacaaatgctatgatttgaaatcaaagggtggaaaagattttgtttccaaatgtgtgggttgttttaaatggggatataacataagtttaatagatcattaactatttgttagctccctgaagtattagtggtagttttaattattcttaataagtgaatctttctagtgtttttaaaagtatatcttttattgtaggaaatttagtgctgagtagtacataaagtattaataccctgattagtattccgatttttcttttaagattgcagcaaaaattgggggtgatgctggtacatcattgaattcaaatgactatggttatgggggacaaaaaagacccttagaagatggaggtaagttatagccaacaagaataattatcttccatggtattcatttgataagattttcagtattgtttatcaagatgatttatcaaaatactactatctattggtctcagaagaataggattttgaaaaagacagacctagaaaaggtaggtataatttgaatctattgggaagtattattgtaatagaaaacaggttttgttatttttttttttttttgtcctgttataggattatgcttgctttgagttgcacagactgttaatgatataaataaagttatgttatttgggaaacactttactgaattttctcatttattgaatgagtctttgatactccctttcattcagtcagaatccacgtctaggttaaggttggttggttgcaggcatcccatttttatattgttttagtgttttgtgtgtttcatatttatctttaaacattgattaaaactgtggattcatagtgttttctctttttaaaaatcatcacttctgcacttagaagtgtgtacttttttattttagttttgatgtatgtataaattttgatattgatatatttgcccttttgtaaaagattctgatagcatataggactgactgtatttgtggccctttctggtaatacttggtttccttttgtttaagtatgtttatatttgcaagtgaggtttttaatatgcaagaaaaaatgagctaatgatgcttcagatgttgtgtgtaatgtactctttttattttatgtgtagatggctcttggacaaatccgagcagtaccacacactgggagggaatgccctctccttttaaaggcaggaatttttatttattacctgtgtttagtatgtaaatgtgaaataagccagtggattcttaaatggacacaatggtctcttggtttatgtgtctgaatttttgctatacaaaagccttgaagttgggagaataataatagtgttttgagctataggctgtctattcatatgttgtatgcattttgtttatttgagaaaggcaatcgtgtggttgctttgaggttttggtgttctgtagtgaaatgcttcatttgagaataatagaacagtttagattgaaattgtcttgtaacgtgagctttcttttttggtatagttttatgatcactgtcagattcactcatcttttgatatgtctggtttttatgtagtgctctctaattgtcagttctagagtggatgaagcatcattaaccattagcataaggtattcaccagcattgaagctaaacagtttgtgctgtattattccaaactaaagtgtctgtgcattgtttaaacacaagagaatgaaagaatttaggttttgtgggttttttttttttttttagatttttgagtctaggttgaattgtgtcagttttgattcaattggtaaaccaatatatcatgtattttaataaatatcattattcttgcttggtgggggtggggagctttgtttttaattgacaaaaagtaaaagacacttaattttgcagatcagccagatgctaagaaagtacctccccaaaatgactgtaagtactttaagctgaggttttgttgcttctttaaaaattacgtgtgtgtgtgtgtgtgtgtgtatgtgtgtgtgtatgtatgtatgtatgtatgtatgtcaagatggcataggaattccctgaaagtggactacatgtgggtgctgagaacttggttctctacaagagcatgtgtcctctttatcatctaagtcttctctccagactgttgtttctactttgggtgggttgtgagtttggggtttggggaatttgcttttgagccaaagtctcaaatatgtaattctgactgcccttgacttcacagagatctacctgtgtaaaagtattttttattttgcctgatgtaattgtcatctctgagccttactgcctcaaaccttctagttctttctgaactctggtgcaaactcctctccaagctgattcaatatagcttctctcttgacctctgccggaattgctctgcttggcctcaaactatccttctcattttctggcttattctgtttttacatgtgtctagcttgttccctctctgcaacctatatatctctacaactgttccaataagactgtcctcccaccccctgggctatttatttatctcttaagtcctcctctctattctcctgagagctagacataattaatcccattctttttggggtttttttttggggggggggggttagacagggtttctctgtgtagccctggctgtcctggaactcactctgtagaccaggctggcctcgaactcagaaatccacctgcctttgcctcccaagtgctgggattaaaggtgtgcgccaccaacgcccggctgtttgtttttcaaaacagggtttctctgtagctctggctgtctagaactcaccctggaccaggctgacctctgcctcccaagtgctgggattaaaaggcgtgacccaccactgcccggctttttttccttttcttttcctcaaagatttgttactttatatatgagtgcactgtagctgccttcaggcacactagaatcggatcctatcccattacagatggttgtgagccaccctgtggttgctgggaattgatctcaggacctctggaagaacagtcagtgctcttaaccactgagccattttcccagccctaattttattcttttgactccttctctgattgatcactttgtctgctactctaaattaaacatcactttcaaacatgagtgcttccttctacaaactaacttcatctttactgtttgggattaaaggtcattaaaggattaaagagcaaagccacaccacaactataagcacatacacccccacccccgtaaataacacaatcttggggctcaccctataatctaagtgatcaaatagcctacaacagacttgcttttgttttctcctgtgcttggactaaggatgtgtgtgccttgccatactcaacagggttggtattacaaatgtctagctggaatttccttacaaatgtatataggtttttactatatttttaaaaaagaatgattctcttttacttgggagccacgtatatacaaacactgatggctgagctgtgactataagttaactactctagttgttaatctgtgtgtccctttcctgactttcttgctactctcagtgatgattcaaatatgaatggtccgaatttttttcctatgtatacatcagggaaagagtttggagtacgtggtatgtttgtctcaaataaactcgaacttaagcttgtttttgttcattttttcgtagcttttggagcacagttacctccaatgcatcagcagcaaaggtatagtcaaaacttttaaagctactttgcaagtcttagttggattgtatgtacagccaagttagtctataatgcaaatgtctgcataccattttgtatgtgtggtttacttctgtaaaccacattgggaaaatggtatagaaacattgttaatttgggaaaacaggttgaaattgacgcatttcagaagaggtaaaataaatgttaaagtggcattcgctttgacaggtgttcagtttcctgccctgctcacacatgctgcctcatttaagaacctggctgggctactgggagactttaagattgggtttattagactgcttttatgtgtatatgtacattatttttctgtgaggaaaatgttagtttctgttctttcttagcagatctgtaatgacagaagaatacaaagtcccagatgggatggtcggatttagtaagtaccattaatactaaggttttaagacaaagcatactttcaaaaatagtatgaatgataacttctcccctttctctaccttaactagtaattggcagaggaggtgaacagatctcacgaatacagcaggaatctggatgcaagatacagatagcacctggtaacattccgtgtgtgtgtgtgtgtgtgtacgaattacttgttgttttgtccagttcttacagttttttgagagtggctaggaaactcaagtattttaagtttctaaatatctgatatttttattgtattttttacttaaacttttctttttatttaattgtagatagtggtggcctaccagaaaggtcttgtatgctaactggaacacctgaatctgtccagtaagtttgaaaatgttaagtctacttacagttaagactgaagactataagcttcgttttcataccatttcgctttaaaagtttacatattatgcctttcttaaaatagggttcatcatctgaacctaagacagagaaattgttaaagccagagactaagccgtaactcaggcatttaggtgcttgccttccatgtaggaagaactgagtttgatcctaaaactaaaaaaaaaaaaactggcgtggtaatatcatggctcaggagttggacacaggcagattttggggacttggtaaccaaccagctaacactatttgatgggttcaaagtcattgagagaccttgcctcaaggtacttatcacaaaaaaggtactgatggttgtaatttgacctatacaccttccatacgtgtctatacagactcagaaattattaaaacctgccttgcacaaatatgttaataacgtcacctaaaactcatgaccaatctagtcatgaaaattttatattcctgtttttctcttccactcaattctaaggctctgataaaactacccaatctttacctctaattgtcttctccccactgccatctcccctccagacagggtttcattgtatatccatagctgactgtctctgaactcactcgctctgtagtccaggctggcctaaaacttaaaagatctgcctgccgaaggcatgtaccaccattgcccaccacactcactgtcttgactgacttgtgttgctgtgaccaagaaagtgttatcatcctacactgaaatcttaacaccgttccttgcatacttttttttttttgtctagtattgagatactgttttacgttcatttgcctgactttctacttcaagaaattcctgactaaactgattttttacttcattcctaaaattttaggagtaatttctaagagtttatagacagatttggaaattacatatcataaaaatatctttgaacctcatgttgtattttgttaacacagtgctttaaaaggtttgtaccattgaagtttcccctttaaaagtttatctagtgtgtgtgcctctcagtttccttcaagctctgcttctaacagctgcatcgacagttctctttccaaggcagaggctgagtcgtgcactagttaaccccgttgctgtgtgacaggatggatggaagatgtttaatgactgtgttcacttggagcatacagacttcagtacttactgcccatttgacttgacactcactcctttggtttaggttcttgcctccgaccatgaaatgccatatgaccaatttagagtatgtttgaaacctgactaagataaaactgctgacttttattattttgggataagttgggttatgctacgtatttttatcaagattaatttgttaacagatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggtattattatattgtaaagaatattattaagtcttttgaggccactttttattgtttttactaacatattttcatgatattaataggaacgggctggtgttaaaatggtaatgattcaagatgggcctcaaaacactggtgctgataaacctcttaggattacgggtgacccatacaaagttcaggtaagcttcactttgtctcttatatataaaggctgagggggtggacaaaatacgtattaaatttttttgacaaatattctgaattggataactttctattatagcaagccaaggaaatggtattagagttaattcgtgatcaaggtggtttcagagaagtgcggaatgagtatggctcaagaataggaggcaatgaagggatagatgtaagtaagaattctgagtcagcagtggttgtatcctaatccatgggcactagtgttttcctgctttgaattaaatggacttggcttaacatcatgtcccttttcttgttttcttccttggggtttttttatcttgttatttttatgatcatttagtatatttacttttatgattaagtggggatgagtttttgagaaatgtcttttaaaatatacttttacttaaggtcccaattccaagatttgctgttggcattgtaataggaagaaatggagaaatgattaaaaaaatacaaaatgatgctggtgttcgaattcagtttaagccaggtaagtttaaggtatatttaatgttctaagaattgatagcttttaatcttgacatttataattggtttgtcacaacttactctgtttttttttgttgttgttgttgttattgttttagatgatggaacaacacctgataggatagcacagataacaggacctccagacaggtgtcagcacgctgcagaaataatcacagaccttctacgaagtgttcaggtgtgataggaaattaacattttcagtttgctttctttggaaaagtttttatcatacctgaatgaaaaaaatgcccctccaggttatattagaatctgttggcagtgttaaaatgactaacgttaaattgctttaaggtaaacattttagggtagatttgcataaatgttctgccttatttcccccagtgcatgtgaacttttatttctatgggtatatgtgccttggcaattgtgcctatggaagccaaaggaaatttttctgaagttggttctctattatgtgggtcctagaaattaaacccaggtcattttgcatgtatgggtagtctcccaatatattttaaaagtggaagttattacttagttgactatttttgtttgacgtcatctcttaatttcattgtttagaaatacgagagttggggataataagctaatagttttattacagagtttattttgttctctcctttcttaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggcaatgtattttaaactctttgttttgatatactggatatttgtaaacaaaagatgctatttatatttttttaggaaaggggtcttaacatggcagctaaaataagaatactctttaatgtatgctaaaaatgggattgtgtctcaactgcttctagggaaaggggaccaaaagtctgtcagttacaaaacttgatcctaattcaaatgggttttgttggtgttttttgttttgttttgcatttaacttctttgttacttacaaaacctgttgattgaaatatttttctgttctgacccatatcagggtgtatgtttagtaaggtgtgtataaaatagactttagtgtgttttgcattagttctagatggaagaacaggctaaatagaaaatcctaacattattgtccagtggaatgctacaaaactgtttaatgggcaaagtaataggtgtttggggttttttttggttttttttaaatagtatttctgccagacatggtgacagacacctttaatcccagcacttagtaggcagatgcaggtgtttctcttgtgagtttgaagccagcatggtctacagggtgagttataggacaaccagggccacacagagacccttgtcttaggaaagagtattgcttgcattctgtgaacttagtgtcttgcggttttaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattggggtaagtgtactctcatttatagcatagttcctggtaactttaaaatgtactgtgggaaaatacatcgtttctaatatgaaattgcaggtattgtgatatagtgtatatacatatattgacataggaaagatatccaaaatacaagaaccatgaactcagaaatccgcctgcctctgcctgtcaagtgctgggattaaaggcgtgcgccaccacacccggctgagaataccatgtgttaagttttcccttgatgtgcaatttgcagaatatactctcagcagttgtcacattagggaagtaaagggaactaggcagtcttgtttttaaaagaaaatcataaaaagagtaagaaacacagaagactaagctgaaagaactctgctggagctgaggatttcagtgaattttacatggcagatgatgacattttattttttgcaccaaaattcaaagagggtaaatttaaatttgttatttaagcgtagaaatggaagagtagttggactttgaattactcaaccattacattgtttgagtgtctagttattgtttatgttttcttttcctttttttttttataatagggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcagtaagtaccacatttgcttggttctgggctttccctaaagacctaggaattgggggctggagagatgacgatggctcagcagtgcagagcacttgttcctgttgcagagggacctgagctcacttcccagcaccacacgggggttcgcaaccattcgtgacaggttccagggtatctttacagcgacactaggcatacatagctgcatatacatacatgcaatacatgcagaggcaaaacctacacataaaataaataaagctggcttttattaatggtatttgtgttttgttccccacccctagcggtcctccagccccatatgctccccagggatggggaaatgcgtatccacattggcagcaacaggctcctcctgacccaggtaaagggtaatctactattaatatgttagcttcattgtatactgctggtcacagtcctaaactacctactgcatgtgcgatcttttaaggcacttcccctgcagtataggatgacactgctgtctagtatgttgtgactttattcatagtagttcatatttataaaataatgtgtttaacactactatgttctcaaataataagtataaattattttgttgctgataaaattaaggtcccttaattaccaaaacaggagagtacatttatttttctctcttataatctggattttctatagcatggtgttctcattatatcttcccatactggttttgtttctactccatcatgatcaggaatcaacttggaaataggacttttttctttgtttttcttttgttttttcactttgcagcccctgtagtttgattcatttgaaaagtttgagagaacgtgttctgagttatagatttctacatagtgtattttttacaaatcaaagttgatttagatgtaattgctttgttggatttgttaaatgttttaatattttaatgtatgttaaattcataggaacttttctcagtcaccaaataaccacaacttctattcatttaagaaacaacttgagccgggcggcagtggtgaacgccattagtcccagcacacaggagacagaagcaggcagatctctgagttcgaggacagcctgatctacagagcatgttcaaggatagccagggacacacaggaaaaatcctatctcaaaaaagagagagagagaaaaaaaaagaaaaagaaataacttgaattgtgatggcatctgaatatgaaagatgagtgtcgattagcaaaagagtgcattcccagggcagacaagaagcatcagttatgacttcctttgtaagggttttaaaagtgaatcttctccttctaacaagcatttagacgtggcatgatcaaatagagttggaacaggttatattgaaggttttatatggctaattcaatttcataggattgctcaaatgagtgatgtttgatattagcactgtaaggtctggtcagtgaatatgacttccagcattacctggaagactgactcggcaatatgttgcttaaatttgacttggaagtatacagtattttattgtctgcattttcaagaaactgctgctgtgaggttgagatttatatagatgtcaagaatttaatcttcaatcttaaatggataatatgcgaattcatcctatgtaaaagaatagtgagctcttagtacctgctagttgcaagtcacaaaaaaaaagtttgggttttggtttggtttttgagttacaaaggtgagcttagtgcagaggatgctggttgtggtgacttgggaatagggcagcagctaaaacttgagtattgggggatgctaaaagaattaatagaaaggataagccacagtgctggggataagcattagaacctgagttctatccctcccacctgttgtaaagaaaaataaataaagcgctttaataaagttcgtaaaaaggatccgtaaagtttcctggcctgctcttgtaaccaaatctaggagctctgggttcagagagaaatcctatctcaaaaaaaattaggaaggaaggctggagagagggctcagtggataagaaaagcaataactgctcttccagaggtctgagttcaattcccagcaaccacatggtagttcacaaccatctgcaatgggatctgatgccctctgctggtgtttctgaagagagcttcaagtgtactcatatacaaaataataataattcttttaaaaaatgaggaagggtgtcagagaggtagcttagtgattattataaaggtcttcctgctctttgagaggatgcaagttgagatccagttactctcacaccttctggttctctttagtaacccacatttgtgtagatacacacaggttcacagatacacataactagaagtgggactttaaagtctagataaaacaaggtgagcatgtctagggagatcacccagaatcagtacacacaaacagacacgccacgacacgatggctaagctggggagggcggccagaccacagcatgtagcccggaaacaggaaaagtggaggaagaaaaggtaacggcttggaaatccaacggcctgtgttaaatggggcatggtcttaaaactgtacaggagctttcaaatgataagctatgcttgaagtatcctaaaggtgttagaaaaggatagagcaggactttcaggattgtgactgaaataaatataaactgcatgtatctaacctttgtttctttcttgtacaaattagtccataactgcctgctatacagaactgttgcacttggcagcttaacttgttgggggaaaaaaagcaaagacacgggattgagctgagacaaaccattggattggaagccaaaagatcatccatttatataccagttccaccgatcactagtaaattacttaatacctgtaacctaggaaattacttaattcccgaggctgaagtttctgaactaaaagccaagtgatgatgataatactagaacattttgaaagtttccattcgctattggtaacttttgagcctatttgtcaggaatcaccaaaaaggtaagggttggtttctagagtttacatattaaccttgaaaccgctctttgtttttacattgtagccaaggcaggagcagatccaaactcggcagcttgggctgcttattatgctcactattaccagcagcaggcacaacccccacctgcagctcctgccggtgcaccagctacaacccaaacgaacggacaaggtaactacggtaatgaaagattttatttcctattttaaagctatagtttgtgtttggattcatgtatgaagtcataacaaaactctctttcagtcctgagaattgttgggccttgtgcatgatggtatcattaggttagatcctcgggcctaaggttttgaagactcagttgtgactttgtgtaagcaaactaagtcaaataatagttggaagaataatgccttaagtagtgagactgttaactgtcctccccccacccccatttacttcactaccctgatttatgtctgttataatagttctgaagagagctactgaaaactgtacttagagacataaaatcatatcacctttctacagacccttattcattccaaaataaatgaccaccatggactttaaagtcagttactcatcattgatataccactgttatacacagcccttgcttagatcaccaactgaccccgtgttttcattaggttcattaagtctcatcaagctagagcactttcttggtcttcttttatgcctctaataagaggataactcaacagttagatagctgccaagctatagagttactaaccatggttgtttaaatggtcttgctactaccagcctggtttttgcactatactacaactatgtactgtaaagaattacctaggggctggtgagatggctcagtggataagagcacccgactgctcttccaaaggtccggagttcaaatcccagcaaccacatggtggctcacaaccatctgtaatgggatacgatccgattctagtgtgcctgaaggcagctacagtgtacttacatataataaataaataaatcttttaaaaaaaaaaagaattacctagtattttctgctactttttactggagtaatgtcactatcctatacattgaacttctgcctcagccttagtctgtattattaataaaccttgccttggtaccctactattacagcagctgttaactgttagtttctgtcattgtatattagttgctattttactacaaggaatagattgcccttttttaactactttcattatattgtcctagatttggggtggttggggttatcttcaaggtggctctggtgcctctgacgttcgttcttcaggtgcttccttagtacctgacacagtaagataactttgtcttctgagctccaatcggtgattcatttccttttctctctctctttctctctctctctctctctctctctctccctctctctctctctctctctctctctctctctcaggatagaatttagagatcagtctaagtatactaattgctatctaaatgtcactaattataatcctgtccaaattagaactaaaaagtatgtttggggctggcattcagtcaatagtacttgttcctgcagaaaacccaggtttgattgctagtacccacatggtaactcacactgaaactgattccaggggacccagtgccctcttctgatctgtggagcaccagatacccagggtgcacagattatacatgtggacaaaatgctcacacataaaattaaacagtatctttaaaaacatgcaggacaacacgtgtctactaaaaatcacatgattatatactactttttcatttaatctttaaaattagtgcttcaaagctatttgtggcttttctaaaaatgtgtgcaggagatatcaagggatgaaccatgcttgaactgtccttaaaaatggatagaaaccatgttctatatttcttttgtgtgcatttacttactattcccagtttctgtgtccaccccttctatacccacttgtcatgtattcccaacaaacacttgcacaactccgtggtgtagacagtgctaattacagtactgatggtgtagaagcaaaagtgcataacaaccttcagaaacttaaatcttgttttgtttgtttgttttttaaaaaggagatcagcaggctccagctccagctggacaggttgattatacaaaggcttgggaagaatactacaagaaaatgggtatgctctacagtttacgtcttatcctttatcttagtaactaaacggaagtaaatacacattacaaagctagagatgagtaacaaaacatttgcttgtttttaaaaaagaatacttgaggtaactcatccgtatgtttttataatgttaaagtttatattaaacaagctaggcagtggtgatacctgcctttaatcccagtacttggaaggtagaggaaggtagatctccgagtttgaggccagccttgtctacaaagcaagttccaggacacttgggactcttagacagagaaaccccatcttgaaaaacaaaaataataataaacaggaaaggtgacatcctgaaattgactggaagttaagacatattaagagcaattcaagaaattcttataattgtgcttatggcagagaaatacaattgtcagcaagttaataattaaaacctggcatttccttactaggtaataatatatggctttattactgtacttttgtgtagtggtctaaatggaatgagattaaattttaggccagcaaagtagttgtatcactggctttactgcaaaataagcataaaattagcccacagtgacattgctttacatttctaaacatttgcatgcattgaaaaggaaaaaagggtgaccttttaaaaagattttgacaactattgaacgtaattcttaacataaccatagtgattgtgttaatgtatttcttgtcggtgatacagctttcgacaaagttcctttcagaacaactgtttggggaaatgtgataacaaacttctgcagtgaacagatgggctgataacttttcttaacctcactgatcgcaggcaggttctaacagatggaccttagcactagaaacttgaaacctgccgggcgtggtggcacacacctttaatcccagcacttggtgggcagaggcaggcagatttctgagttcgaggacagcctggtctacagagtgagttccatccaggacagccagggctacacagagaaaccctgtttggggcctggggggaagaaacttgaaaccttaaaacaatattgtgtgtaattagaatgcttacataatttcttctctgaactgtgatatttttgagagtagatggaaatgtattaatcattccgtggtagccgtgctgaaatagtgctacttgtttataagaagctgagcatgccaaggtgaaagtgcgctaactccattgtttgtttttactgtgtgagtgttgattgggtagaaacctggtaactggactacgtaattaagacagagtaaggtatttgtctgtgctcgattatgtcatgttatttgtttacatgacttcaaataagttttatagatattttgcattctgtattcttaaaaagtatattgactaggacttttgagagtaggtattttaactcaattaggttttagtttttaatattctagcactttccagcacacagttctgtgaattcctcttcttctaattattattattattattagagatttatttattttatgtatatgaatgcactgtagctgtacagatggttgtgagccttcatatggttgttggaaattgaattttaagaactctgctcaatccgacccaaagatttatttactgtcataaataagtacaccgttgctgacttcagacataccagaagagggcatcatatctcactatgggtggttgtgagctaccatgtggttgctaggatttgaactcaggaccttgagaagagtagtactcttactcgctgagccgtctcccagcccgaattcctgttcttggttgttatgtctaccgatcaatcagttggcatacatagagacagacacacacagacagcagcatcgtgcaggataactatttggagactcattttcttcatttgtgcttttttgtaattatgttgaagatcttaatcctgtgagcaaaaaaagactaactgaacaattccctgtcatatagatcttgaaagatactgttttaggtaatttaattaatcattgaattagtatttttaattatatgatagttataaaaatcatttttccctgtaaaacatttaactacaatttttttctgaaagtatggtccttgatatattttttagtataatcatgaattaagagaccttaaagcaaaaggttcagaatagaagctcaggagatagatgggtaagagtgcttaatgacctgggttcaaatcccactacctatcttaaaagccaatcatgactgttgcatgttgggaagcagttgcaggagcttgctagccaactgtggattcagtaaaaggagcaggggaataaggtacagagggatagaagaaggcaaaggcaggcagatttctgagttcgaggccagcctggtctgcaaagtgagttccaggacagccagggctatacagagaaaccctgtctcgaaaaaaaagaaaaagaaactcatttgcagttaacttgcttgtttacaaaattcattaagtatttctagaattcgatttttatcatttgataactcagaatggtcacttaaaacaaaattactaataaacgctgagttttgggctggtgagatcgctcagtgggtaagagcaccctactgctcgtccgaaggtccggagttcaaatcccagcaaccacatggtggctcacaacaaccatctgtaacaagatctgacaccctcttctggagtgtctgaagacagctacagtgtacttacatataataaataaataaatctttaaaaaaaaaaagctgagttttacatctttcattgaactttatttcagctgccaggttatttttagtaaagctgtgatataaactcagtatgtcttttgcttcattgtgtgtgtcataaatatctttcagtacttctctgtagtttaagactagtaatatagtgacccaaatggatagaagagtggtgagctaggtatggtggcacatatacgcatttagtcctagtgctcaggaggcagaagcaggtagatctccaaggatgaggtcagccctggatagtgagaccctttctcagttagcaaaaaaaaaaaacaaagcgtttcttctttccttgtcaaagattatatctgaattttcccctttgtttaaaggtatgtgagtttgggtcattttgtcttttactaatcaagctgaactaagagactgaaacttgtgattgaagtgaaaagagtatctttgccgtttcttgtgtgctgcactttcccccctcatcctccctcccttgtctaaggcatatttaagataaacagaaactgaaggggcaatgaggcacagataatcctagcacgtgggaagttgccaagaggataacaagttttgagccacctgtgctacataagtgaaaccctgtgttgagataaaaacaagagctattgcccctctgtgccttgcattataagctgtacttaatggtacatacatttttgtattaaagttgagggggtgttttaggtcacaagggcagacacgaggttattaaaaggcttggaagaagtattaaaaaggcaagatgctgtgtttactagagaagagttactgtgagatttgtaattatagcagcaataaagttctctgttttcctgaaggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcaggtagaaaacatttgctattttttttgttttgtttttgtttttgttttccactccaaccatgttttctgcatgtttgtctgtttgggaatgaatatgttttacttggtaaagtataactacatgaaatatcgtgttgtgtttggggactcagtttttctaataatgttttctggcatctgagtgctatttttgtaatgcctttgattttaaaaataaattttcttccccccagggatttgcaaatcatgcaagaagccaccatcatttatattaaccagtttttctttcttaaaggattcactcctgaattagctccatttaaaggattttctttaactttttgtgtatctcttatgtatctcttctgcacagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcctgtgtcggtaagttggatatgtgggtaaaggattcatagtttcttagcagtgataaattaagatacatgtacacgaatatataagctttccccatgaattactgagtttttaaacactggcatgttttttcccctgttggagtatagtggtagattggaggttcttttctgttgtatttggctatttcagcacaagtaatcctgatattttcatgtttttccttctatttgattaaaaactgcatgtgtatacaatgatctttagtatacttccattgcattaacagtgacatttccttttatacatgagcactatttcagacctgtaccgctgctacaacagttaaccttcctgttcttcacttatttccgagactgtttcagcataactaattttgaacagtttgcagacagtgatttgaggagtttataagaaacattgtttttttcatgtaaagcaactctttccatgtatatatatatattataatagtgtgtttctctctaaattcaggatagaaaagtaatagaatgtgaaagtatagctacattgcatctgccattgaaacatttggggtatgaaaatgttcaagctttttttttctttttgcagtatagataagctttgtcttgtaattgcacaagtccagtcattgaatcaaattaatttttttatgtactgaatcattttattaatctttaacattcatgctgaagttctgatattttgttgaaaaccattgttttactctgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagagtgacaaattgatgaactgcaacagtgtctttttgtcgatgttccacataatgcaattgctatattttgtgtgactattatgttggaatacagtgctgtcatgggaaaccataactgcttcttaacatagaataatacatagttctgtattttttttaagtgagcttaatgggtaagtatttttgatatgctttagctaatagctaaagaaaattgatcagtaacaaagttgaatagtattatcagtgctcctaaaatgctgtttttcagtgtaaaatatgcctatcttttatagtgatatgaaaaacttgaaatgtttaagacagaagtgcttttcagtttgcaaagtttaaggacttaacgccttttcattaaatgttagttctatcatctgtggggagcaccatttgtatgaggacaaaaatgttgaggttatagggcagaaaatagtacagctcattgtggatgttttgaaatgttttttgattgttttatgtaacttagtgacttcccttccccttatttagatctggatgcatagctctagtatgaagtttaatagtaatagttaaggccttacctgttaagatcttaagtgtagggtattgacatgaacagtttcagtattttgcatgatattgttgcatagatgacctaggaaagtgttgtggtgcatttagtaattaaactgaaggaaatagttggattcagtatcataattcacaaattggaggctgttgattttgattcgtttaaaatttaaaatctttattaattgcaaacagtgcaattatttatacttcacagtgccttcccagaccttccaccttaggttctgctgcaaaaagcaacaggtaagcacaacctaaggccatctctatataaatatatcagtacatacatgttgtccctgtgaggtttgtggttgtgtactcactcaagcaatctgctgctgccgctgccccaaatgtactttgttatttatggtaccattctagtggaaagtctgttaagttgttcaagcaactgtttacaattttgggtgatgttttgtttttggtttttttgggttttttttttccccattaaaatgagtagattgctgcaataactgaaaaacatccaaatctttttgtgcacccctcctctcccagagttatagaaatgtttaagaacacttcagtagttgagacataggaaatcatttggtcagaaatttcaacagccttcacagcccgtttgtatgagtaacaggaaattctctttgccctccaaatctgatctttttatagttttatttttattatttttcccctgtattagtactgctttaaagtacagtaattcagtgagatcgttttggttcagtttcatagacagttctattttcatgacttgtatttgctgacaaggaagaataaagaatactggcagtaagttttgggaaaggcatgtgcatcagtgaaatgttacttgtataacaaataacctttcataatctgcataaccagtagtttctgatttataatattttaataactgactgcatttatttttgccagtttaaaatgttttgtgttcttaggattgatgtttagtgcatattttgagttaaatgactatcttaaagcagcaccatatcagttgtttttattcattttctaaaatgtgctgatcctattaaaaactcctgcttatcttttacaacaaagaaaaatattcaaaaatactgccttcattttcacacacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgactgtgttcccatacataatatggtatagggtgtaatgtacctgctcttgatcactttcattttaaagtgctattcacttaaatgttccatgaattgtttattgcaccacagtttgtgtgtgtgtgtgtgtgtgagagagagagagagagagagagaaataggtcagatcagtatgttgcaagcagatctttagagcctgtcaagttttggttagttgtagtttccatttggaaatgtagatgaatgcttgtagatattggagattgtttctatcttgtaagaacttttcactggtgctgtaagcatttcaaatagcaccagtcttaacctttaaatgggaagtagaaaaggtgagcccaaagtttacagatgattttaatgctatacatgttagtgtagtgatacttagaatgctttgtttgatgtttatttcagaaatgcgtatactagaaaatcattttaatattaaaactggtgacttaatactagttgtaaagtgttttcttaaagaaggatcttggtacttaattgataaagtgggtttagataggagtagcaagtgctctcgatagagaaagtttttgttcacttcaatactttgtcattacaaccagttcttcctgaaaatagttacatgtctagtaaattgatgtagaattaactcgctggagataatctcattttagttctgcaaattctgcctggctttaaaatgcattttcataatacttagaaataatttgactgaaaataactgcttttttatttaatcagtcaatcaacttttactacaaatttaattgagggattttttaatttaattggtgctttaaagaagcaaataaatccctgggttttgttttcttcagtaaatatcctaaagaaactctttaatgtatttgcgagtatatatatattttcttatgcatgctcgatgcattttcgtcctgagaaaagtgttctctacagaaactacccgtgtgttaaaagaagattggcttaaaatggctactgtgatgggaacagtgtcttagggagatgcagcttggacttgaggtaaattgaatactttacaaactggcttagagttttgctttaatgtcattatatgtaaaagggcacatgattattgtaattttgtattctttatggtttccttaattaaaataataaatgtacagtgattact

SEQ ID NO: 10 - DNA, 4525 bp, Mus musculus
  source 1..4525: mol_type = genomic DNA
cctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggtaactacggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcctgtgtcggtaagttggatatgtgggtaaaggattcatagtttcttagcagtgataaattaagatacatgtacacgaatatataagctttccccatgaattactgagtttttaaacactggcatgttttttcccctgttggagtatagtggtagattggaggttcttttctgttgtatttggctatttcagcacaagtaatcctgatattttcatgtttttccttctatttgattaaaaactgcatgtgtatacaatgatctttagtatacttccattgcattaacagtgacatttccttttatacatgagcactatttcagacctgtaccgctgctacaacagttaaccttcctgttcttcacttatttccgagactgtttcagcataactaattttgaacagtttgcagacagtgatttgaggagtttataagaaacattgtttttttcatgtaaagcaactctttccatgtatatatatatattataatagtgtgtttctctctaaattcaggatagaaaagtaatagaatgtgaaagtatagctacattgcatctgccattgaaacatttggggtatgaaaatgttcaagctttttttttctttttgcagtatagataagctttgtcttgtaattgcacaagtccagtcattgaatcaaattaatttttttatgtactgaatcattttattaatctttaacattcatgctgaagttctgatattttgttgaaaaccattgttttactctgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagagtgacaaattgatgaactgcaacagtgtctttttgtcgatgttccacataatgcaattgctatattttgtgtgactattatgttggaatacagtgctgtcatgggaaaccataactgcttcttaacatagaataatacatagttctgtattttttttaagtgagcttaatgggtaagtatttttgatatgctttagctaatagctaaagaaaattgatcagtaacaaagttgaatagtattatcagtgctcctaaaatgctgtttttcagtgtaaaatatgcctatcttttatagtgatatgaaaaacttgaaatgtttaagacagaagtgcttttcagtttgcaaagtttaaggacttaacgccttttcattaaatgttagttctatcatctgtggggagcaccatttgtatgaggacaaaaatgttgaggttatagggcagaaaatagtacagctcattgtggatgttttgaaatgttttttgattgttttatgtaacttagtgacttcccttccccttatttagatctggatgcatagctctagtatgaagtttaatagtaatagttaaggccttacctgttaagatcttaagtgtagggtattgacatgaacagtttcagtattttgcatgatattgttgcatagatgacctaggaaagtgttgtggtgcatttagtaattaaactgaaggaaatagttggattcagtatcataattcacaaattggaggctgttgattttgattcgtttaaaatttaaaatctttattaattgcaaacagtgcaattatttatacttcacagtgccttcccagaccttccaccttaggttctgctgcaaaaagcaacagtgctgaagatgctgcaagcaccaaatcatagctcataaaatcaggtcctgagatagttacccataaagaggaatcctttgagtgtatgccattggtgagccgatgagcatggaccatagaagggctcaatgtagaagaaactacccgtgtgttaaaagaagattggcttaaaatggctactgtgatgggaacagtgtcttagggagatgcagcttggacttgaggtaaattgaatactttacaaactggcttagagttttgctttaatgtcattatatgtaaaagggcacatgattattgtaattttgtattctttatggtttccttaattaaaataataaatgtacagtgattact

SEQ ID NO: 11 - DNA, 800 bp, Mus musculus
  source 1..800: mol_type = genomic DNA
cagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcctgtgtcggtaagttgga

SEQ ID NO: 12 - DNA, 2526 bp, Mus musculus
  source 1..2526: mol_type = genomic DNA
gcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagcagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggatttgcaaatcatgcaagaagccaccatcatttatattaaccagtttttctttcttaaaggattcactcctgaattagctccatttaaaggattttctttaactttttgtgtatctcttatgtatctcttctgcacagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctc

SEQ ID NO: 13 - DNA, 809 bp, Mus musculus
  source 1..809: mol_type = genomic DNA
gaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagtgccttcccagaccttccaccttaggttctgctgcaaaaagcaacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgactgtgttcccatacataatatggtatagggtgtaatgtacctgctcttgatcactttcattttaaagtgctattcacttaaatgttccatgaattgtttattgcaccacagtttgtgtgtgtgtgtgtgtgtgagagagagagagagagagagagaaataggtcagatcagtatgttgcaagcagatctttagagcc

SEQ ID NO: 14 - DNA, 1040 bp, Mus musculus
  source 1..1040: mol_type = genomic DNA
gatggtcgtgcaagaatgtgatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatggctcttggacaaatccgagcagtaccacacactgggagggaatgccctctccttttaaagatcagccagatgctaagaaagtacctccccaaaatgactcttttggagcacagttacctccaatgcatcagcagcaaagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaat

SEQ ID NO: 15 - DNA, 796 bp, Mus musculus
  source 1..796: mol_type = genomic DNA
cccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcc

SEQ ID NO: 16 - DNA, 585 bp, Mus musculus
  source 1..585: mol_type = genomic DNA
aatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagaaacaacttgagccgggcggcagtggtgaacgccattagtcccagcacacaggagacagaagcaggcagatctctgagttcgaggacagcctgatctacagagcatgttcaaggatagccagggacacacaggaaaaatcctatct

SEQ ID NO: 17 - DNA, 2374 bp, Mus musculus
  source 1..2374: mol_type = genomic DNA
ttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagcagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggtaactacggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctc

SEQ ID NO: 18 - DNA, 3163 bp, Mus musculus
  source 1..3163: mol_type = genomic DNA
gatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccaggtaaagggtaatctactattaatatgttagcttcattgtatactgctggtcacagtcctaaactacctactgcatgtgcgatcttttaaggcacttcccctgcagtataggatgacactgctgtctagtatgttgtgactttattcatagtagttcatatttataaaataatgtgtttaacactactatgttctcaaataataagtataaattattttgttgctgataaaattaaggtcccttaattaccaaaacaggagagtacatttatttttctctcttataatctggattttctatagcatggtgttctcattatatcttcccatactggttttgtttctactccatcatgatcaggaatcaacttggaaataggacttttttctttgtttttcttttgttttttcactttgcagcccctgtagtttgattcatttgaaaagtttgagagaacgtgttctgagttatagatttctacatagtgtattttttacaaatcaaagttgatttagatgtaattgctttgttggatttgttaaatgttttaatattttaatgtatgttaaattcataggaacttttctcagtcaccaaataaccacaacttctattcatttaagaaacaacttgagccgggcggcagtggtgaacgccattagtcccagcacacaggagacagaagcaggcagatctctgagttcgaggacagcctgatctacagagcatgttcaaggatagccagggacacacaggaaaaatcctatctcaaaaaagagagagagagaaaaaaaaagaaaaagaaataacttgaattgtgatggcatctgaatatgaaagatgagtgtcgattagcaaaagagtgcattcccagggcagacaagaagcatcagttatgacttcctttgtaagggttttaaaagtgaatcttctccttctaacaagcatttagacgtggcatgatcaaatagagttggaacaggttatattgaaggttttatatggctaattcaatttcataggattgctcaaatgagtgatgtttgatattagcactgtaaggtctggtcagtgaatatgacttccagcattacctggaagactgactcggcaatatgttgcttaaatttgacttggaagtatacagtattttattgtctgcattttcaagaaactgctgctgtgaggttgagatttatatagatgtcaagaatttaatcttcaatcttaaatggataatatgcgaattcatcctatgtaaaagaatagtgagctcttagtacctgctagttgcaagtcacaaaaaaaaagtttgggttttggtttggtttttgagttacaaaggtgagcttagtgcagaggatgctggttgtggtgacttgggaatagggcagcagctaaaacttgagtattgggggatgctaaaagaattaatagaaaggataagccacagtgctggggataagcattagaacctgagttctatccctcccacctgttg

SEQ ID NO: 19 - DNA, 6523 bp, Mus musculus
  source 1..6523: mol_type = genomic DNA
gctgaaacatcgcgggatttccgtctgccagtctccgcccctttacggcacgatggtcgtgcaagaatgtgatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggtaactacggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcctgtgtcggtaagttggatatgtgggtaaaggattcatagtttcttagcagtgataaattaagatacatgtacacgaatatataagctttccccatgaattactgagtttttaaacactggcatgttttttcccctgttggagtatagtggtagattggaggttcttttctgttgtatttggctatttcagcacaagtaatcctgatattttcatgtttttccttctatttgattaaaaactgcatgtgtatacaatgatctttagtatacttccattgcattaacagtgacatttccttttatacatgagcactatttcagacctgtaccgctgctacaacagttaaccttcctgttcttcacttatttccgagactgtttcagcataactaattttgaacagtttgcagacagtgatttgaggagtttataagaaacattgtttttttcatgtaaagcaactctttccatgtatatatatatattataatagtgtgtttctctctaaattcaggatagaaaagtaatagaatgtgaaagtatagctacattgcatctgccattgaaacatttggggtatgaaaatgttcaagctttttttttctttttgcagtatagataagctttgtcttgtaattgcacaagtccagtcattgaatcaaattaatttttttatgtactgaatcattttattaatctttaacattcatgctgaagttctgatattttgttgaaaaccattgttttactctgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagagtgacaaattgatgaactgcaacagtgtctttttgtcgatgttccacataatgcaattgctatattttgtgtgactattatgttggaatacagtgctgtcatgggaaaccataactgcttcttaacatagaataatacatagttctgtattttttttaagtgagcttaatgggtaagtatttttgatatgctttagctaatagctaaagaaaattgatcagtaacaaagttgaatagtattatcagtgctcctaaaatgctgtttttcagtgtaaaatatgcctatcttttatagtgatatgaaaaacttgaaatgtttaagacagaagtgcttttcagtttgcaaagtttaaggacttaacgccttttcattaaatgttagttctatcatctgtggggagcaccatttgtatgaggacaaaaatgttgaggttatagggcagaaaatagtacagctcattgtggatgttttgaaatgttttttgattgttttatgtaacttagtgacttcccttccccttatttagatctggatgcatagctctagtatgaagtttaatagtaatagttaaggccttacctgttaagatcttaagtgtagggtattgacatgaacagtttcagtattttgcatgatattgttgcatagatgacctaggaaagtgttgtggtgcatttagtaattaaactgaaggaaatagttggattcagtatcataattcacaaattggaggctgttgattttgattcgtttaaaatttaaaatctttattaattgcaaacagtgcaattatttatacttcacagtgccttcccagaccttccaccttaggttctgctgcaaaaagcaacaggtaagcacaacctaaggccatctctatataaatatatcagtacatacatgttgtccctgtgaggtttgtggttgtgtactcactcaagcaatctgctgctgccgctgccccaaatgtactttgttatttatggtaccattctagtggaaagtctgttaagttgttcaagcaactgtttacaattttgggtgatgttttgtttttggtttttttgggttttttttttccccattaaaatgagtagattgctgcaataactgaaaaacatccaaatctttttgtgcacccctcctctcccagagttatagaaatgtttaagaacacttcagtagttgagacataggaaatcatttggtcagaaatttcaacagccttcacagcccgtttgtatgagtaacaggaaattctctttgccctccaaatctgatctttttatagttttatttttattatttttcccctgtattagtactgctttaaagtacagtaattcagtgagatcgttttggttcagtttcatagacagttctattttcatgacttgtatttgctgacaaggaagaataaagaatactggcagtaagttttgggaaaggcatgtgcatcagtgaaatgttacttgtataacaaataacctttcataatctgcataaccagtagtttctgatttataatattttaataactgactgcatttatttttgccagtttaaaatgttttgtgttcttaggattgatgtttagtgcatattttgagttaaatgactatcttaaagcagcaccatatcagttgtttttattcattttctaaaatgtgctgatcctattaaaaactcctgcttatcttttacaacaaagaaaaatattcaaaaatactgccttcattttcacacacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgactgtgttcccatacataatatggtatagggtgtaatgtacctgctcttgatcactttcattttaaagtgctattcacttaaatgttccatgaattgtttattgcaccacagtttgtgtgtgtgtgtgtgtgtgagagagagagagagagagagagaaataggtcagatcagtatgttgcaagcagatctttagagcctgtcaagttttggttagttgtagtttccatttggaaatgtagatgaatgcttgtagatattggagattgtttctatcttgtaagaacttttcactggtgctgtaagcatttcaaatagcaccagtcttaacctttaaatgggaagtagaaaaggtgagcccaaagtttacagatgattttaatgctatacatgttagtgtagtgatacttagaatgctttgtttgatgtttatttcagaaatgcgtatactagaaaatcattttaatattaaaactggtgacttaatactagttgtaaagtgttttcttaaagaaggatcttggtacttaattgataaagtgggtttagataggagtagcaagtgctctcgatagagaaagtttttgttcacttcaatactttgtcattacaaccagttcttcctgaaaatagttacatgtctagtaaattgatgtagaattaactcgctggagataatctcattttagttctgcaaattctgcctggctttaaaatgcattttcataatacttagaaataatttgactgaaaataactgcttttttatttaatcagtcaatcaacttttactacaaatttaattgagggattttttaatttaattggtgctttaaagaagcaaataaatccctgggttttgttttcttcagtaaatatcctaaagaaactctttaatgtatttgcgagtatatatatattttcttatgcatgctcgatgcattttcgtcctgagaaaagtgttctctacagaaactacccgtgtgttaaaagaagattggcttaaaatggctactgtgatgggaacagtgtcttagggagatgcagcttggacttgaggtaaattgaatactttacaaactggcttagagttttgctttaatgtcattatatgtaaaagggcacatgattattgtaattttgtattctttatggtttccttaattaaaataataaatgtacagtgattact

SEQ ID NO: 20 - DNA, 2552 bp, Mus musculus
  source 1..2552: mol_type = genomic DNA
gcaagaatgtgatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagcagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggtaactacggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggatttgcaaatcatgcaagaagccaccatcatttatattaaccagtttttctttcttaaaggattcactcctgaattagctccatttaaaggattttctttaactttttgtgtatctcttatgtatctcttctgcacagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcc

SEQ ID NO: 21 - RNA, 19 nt, Homo sapiens
  source 1..19: mol_type = genomic RNA
gacaaacctcttaggatta

SEQ ID NO: 22 - RNA, 19 nt, Homo sapiens
  source 1..19: mol_type = genomic RNA
gagaagttcggaatgagta

SEQ ID NO: 23 - RNA, 19 nt, Homo sapiens
  source 1..19: mol_type = genomic RNA
gaaaggatagcacaaataa

SEQ ID NO: 24 - RNA, 19 nt, Homo sapiens
  source 1..19: mol_type = genomic RNA
aataagaagtggacaatac

SEQ ID NO: 25 - DNA, 21 bp, synthetic construct
  misc_feature 1..21: note = primer
  source 1..21: mol_type = other DNA
tgtccagtcagcaaaacggtt

SEQ ID NO: 26 - DNA, 21 bp, synthetic construct
  misc_feature 1..21: note = primer
  source 1..21: mol_type = other DNA
gaactgcatttcccggtccat

SEQ ID NO: 27 - DNA, 21 bp, synthetic construct
  misc_feature 1..21: note = primer
  source 1..21: mol_type = other DNA
ggagtgtggattcgcactcct

SEQ ID NO: 28 - DNA, 20 bp, synthetic construct
  misc_feature 1..20: note = primer
  source 1..20: mol_type = other DNA
agattgagatcttctgcgac

SEQ ID NO: 29 - DNA, 27 bp, synthetic construct
  misc_feature 1..27: note = Probe
  source 1..27: mol_type = other DNA
aggcaggtcccctagaagaagaactcc

SEQ ID NO: 30 - DNA, 19 bp, synthetic construct
  misc_feature 1..19: note = primer
  source 1..19: mol_type = other DNA
ccgtgtgcacttcgcttca

SEQ ID NO: 31 - DNA, 19 bp, synthetic construct
  misc_feature 1..19: note = primer
  source 1..19: mol_type = other DNA
gcacagcttggaggcttga

SEQ ID NO: 32 - DNA, 23 bp, synthetic construct
  misc_feature 1..23: note = Probe
  source 1..23: mol_type = other DNA
catggagaccaccgtgaacgccc

SEQ ID NO: 33 - DNA, 15 bp, synthetic construct
  misc_feature 1..15: note = oligonucleotide motif
  source 1..15: mol_type = other DNA
ctcattccgaacttc

SEQ ID NO: 34 - DNA, 18 bp, synthetic construct
  misc_feature 1..18: note = Synthetic oligonucleotide
  source 1..18: mol_type = other DNA
ataaccatagtcatttga

SEQ ID NO: 35 - DNA, 17 bp, synthetic construct
  misc_feature 1..17: note = Synthetic oligonucleotide
  source 1..17: mol_type = other DNA
cccataaccatagtcat

SEQ ID NO: 36 - DNA, 18 bp, synthetic construct
  misc_feature 1..18: note = Synthetic oligonucleotide
  source 1..18: mol_type = other DNA
gagccatctacacataaa

SEQ ID NO: 37 - DNA, 18 bp, synthetic construct
  misc_feature 1..18: note = Synthetic oligonucleotide
  source 1..18: mol_type = other DNA
caagagccatctacacat

SEQ ID NO: 38 - DNA, 18 bp, synthetic construct
  misc_feature 1..18: note = Synthetic oligonucleotide
  source 1..18: mol_type = other DNA
tgtccatttaagaatcca

SEQ ID NO: 39 - DNA, 18 bp, synthetic construct
  misc_feature 1..18: note = Synthetic oligonucleotide
  source 1..18: mol_type = other DNA
ttgtgtccatttaagaat

SEQ ID NO: 40 - DNA, 16 bp, synthetic construct
  misc_feature 1..16: note = Synthetic oligonucleotide
  source 1..16: mol_type = other DNA
tacctttgctgctgat

SEQ ID NO: 41 - DNA, 17 bp, synthetic construct
  misc_feature 1..17: note = Synthetic oligonucleotide
  source 1..17: mol_type = other DNA
gactatacctttgctgc

SEQ ID NO: 42 - DNA, 20 bp, synthetic construct
  misc_feature 1..20: note = Synthetic oligonucleotide
  source 1..20: mol_type = other DNA
actttgtattcttctgtcat

SEQ ID NO: 43 - DNA, 18 bp, synthetic construct
  misc_feature 1..18: note = Synthetic oligonucleotide
  source 1..18: mol_type = other DNA
actttgtattcttctgtc

SEQ ID NO: 44 - DNA, 19 bp, synthetic construct
  misc_feature 1..19: note = Synthetic oligonucleotide
  source 1..19: mol_type = other DNA
gattcaggtgttccagtta

SEQ ID NO: 45 - DNA, 17 bp, synthetic construct
  misc_feature 1..17: note = Synthetic oligonucleotide
  source 1..17: mol_type = other DNA
ttcaaacttactggaca

SEQ ID NO: 46 - DNA, 17 bp, synthetic construct
  misc_feature 1..17: note = Synthetic oligonucleotide
  source 1..17: mol_type = other DNA
tacatctatcccttcat

SEQ ID NO: 47 - DNA, 16 bp, synthetic construct
  misc_feature 1..16: note = Synthetic oligonucleotide
  source 1..16: mol_type = other DNA
ttacatctatcccttc

SEQ ID NO: 48 - DNA, 20 bp, synthetic construct
  misc_feature 1..20: note = Synthetic oligonucleotide
  source 1..20: mol_type = other DNA
cttcctattacaatgccaac

SEQ ID NO: 49 - DNA, 19 bp, synthetic construct
  misc_feature 1..19: note = Synthetic oligonucleotide
  source 1..19: mol_type = other DNA
atttcttcctattacaatg

SEQ ID NO: 50 - DNA, 19 bp, synthetic construct
  misc_feature 1..19: note = Synthetic oligonucleotide
  source 1..19: mol_type = other DNA
ccatttcttcctattacaa

SEQ ID NO: 51 - DNA, 17 bp, synthetic construct
  misc_feature 1..17: note = Synthetic oligonucleotide
  source 1..17: mol_type = other DNA
gtttaaaatacattgcc

SEQ ID NO: 52 - DNA, 19 bp, synthetic construct
  misc_feature 1..19: note = Synthetic oligonucleotide
  source 1..19: mol_type = other DNA
gagtttaaaatacattgcc

SEQ ID NO: 53 - DNA, 18 bp, synthetic construct
  misc_feature 1..18: note = Synthetic oligonucleotide
  source 1..18: mol_type = other DNA
gctttttatggtttcacc

SEQ ID NO: 54 - DNA, 20 bp, synthetic construct
  misc_feature 1..20: note = Synthetic oligonucleotide
  source 1..20: mol_type = other DNA
atgctttttatggtttcacc

SEQ ID NO: 55 - DNA, 19 bp, synthetic construct
  misc_feature 1..19: note = Synthetic oligonucleotide
  source 1..19: mol_type = other DNA
ataatcaacctgtccagct

SEQ ID NO: 56 - DNA, 18 bp, synthetic construct
  misc_feature 1..18: note = Synthetic oligonucleotide
  source 1..18: mol_type = other DNA
ataatcaacctgtccagc

SEQ ID NO: 57 - DNA, 17 bp, synthetic construct
  misc_feature 1..17: note = Synthetic oligonucleotide
  source 1..17: mol_type = other DNA
ataatcaacctgtccag

SEQ ID NO: 58 - DNA, 18 bp, synthetic construct
  misc_feature 1..18: note = Synthetic oligonucleotide
  source 1..18: mol_type = other DNA
tataatcaacctgtccag

SEQ ID NO: 59 - DNA, 19 bp, synthetic construct
  misc_feature 1..19: note = Synthetic oligonucleotide
  source 1..19: mol_type = other DNA
gtataatcaacctgtccag

SEQ ID NO: 60 - DNA, 16 bp, synthetic construct
  misc_feature 1..16: note = Synthetic oligonucleotide
  source 1..16: mol_type = other DNA
ataatcaacctgtcca

SEQ ID NO: 61 - DNA, 17 bp, synthetic construct
  misc_feature 1..17: note = Synthetic oligonucleotide
  source 1..17: mol_type = other DNA
tataatcaacctgtcca

SEQ ID NO: 62 - DNA, 18 bp, synthetic construct
  misc_feature 1..18: note = Synthetic oligonucleotide
  source 1..18: mol_type = other DNA
gtataatcaacctgtcca

SEQ ID NO: 63 - DNA, 17 bp, synthetic construct
  misc_feature 1..17: note = Synthetic oligonucleotide
  source 1..17: mol_type = other DNA
gtataatcaacctgtcc

SEQ ID NO: 64 - DNA, 16 bp, synthetic construct
  misc_feature 1..16: note = Synthetic oligonucleotide
  source 1..16: mol_type = other DNA
tataatcaacctgtcc

SEQ ID NO: 65 - DNA, 17 bp, synthetic construct
  misc_feature 1..17: note = Synthetic oligonucleotide
  source 1..17: mol_type = other DNA
cccattttcttgtagta

SEQ ID NO: 66 - DNA, 18 bp, synthetic construct
  misc_feature 1..18: note = Synthetic oligonucleotide
  source 1..18: mol_type = other DNA
acccattttcttgtagta

SEQ ID NO: 67 - DNA, 19 bp, synthetic construct
  misc_feature 1..19: note = Synthetic oligonucleotide
  source 1..19: mol_type = other DNA
tacccattttcttgtagta

SEQ ID NO: 68 - DNA, 20 bp, synthetic construct
  misc_feature 1..20: note = Synthetic oligonucleotide
  source 1..20: mol_type = other DNA
atacccattttcttgtagta

SEQ ID NO: 69 - DNA, 18 bp, synthetic construct
  misc_feature 1..18: note = Synthetic oligonucleotide
  source 1..18: mol_type = other DNA
atacccattttcttgtag

SEQ ID NO: 70 - DNA, 19 bp, synthetic construct
  misc_feature 1..19: note = Synthetic oligonucleotide
  source 1..19: mol_type = other DNA
catacccattttcttgtag

SEQ ID NO: 71 - DNA, 18 bp, synthetic construct
  misc_feature 1..18: note = Synthetic oligonucleotide
  source 1..18: mol_type = other DNA
catacccattttcttgta

SEQ ID NO: 72 - DNA, 18 bp, synthetic construct
  misc_feature 1..18: note = Synthetic oligonucleotide
  source 1..18: mol_type = other DNA
tggagctaattcaggagt

SEQ ID NO: 73 - DNA, 20 bp, synthetic construct
  misc_feature 1..20: note = Synthetic oligonucleotide
  source 1..20: mol_type = other DNA
aaatggagctaattcaggag

SEQ ID NO: 74 - DNA, 19 bp, synthetic construct
  misc_feature 1..19: note = Synthetic oligonucleotide
  source 1..19: mol_type = other DNA
ttgtccacttcttattatt

SEQ ID NO: 75 - DNA, 18 bp, synthetic construct
  misc_feature 1..18: note = Synthetic oligonucleotide
  source 1..18: mol_type = other DNA
ccccacacaatgaagcaa

SEQ ID NO: 76 - DNA, 19 bp, synthetic construct
  misc_feature 1..19: note = Synthetic oligonucleotide
  source 1..19: mol_type = other DNA
ttcatcaagtcgtctgcat

SEQ ID NO: 77 - DNA, 18 bp, synthetic construct
  misc_feature 1..18: note = Synthetic oligonucleotide
  source 1..18: mol_type = other DNA
gatcttcatcaagtcgtc

SEQ ID NO: 78 - DNA, 19 bp, synthetic construct
  misc_feature 1..19: note = Synthetic oligonucleotide
  source 1..19: mol_type = other DNA
atattaacctcctatcagt

SEQ ID NO: 79 - DNA, 19 bp, synthetic construct
  misc_feature 1..19: note = Synthetic oligonucleotide
  source 1..19: mol_type = other DNA
aatattaacctcctatcag

SEQ ID NO: 80 - DNA, 19 bp, synthetic construct
  misc_feature 1..19: note = Synthetic oligonucleotide
  source 1..19: mol_type = other DNA
atttatatcacaaagcatc

SEQ ID NO: 81 - DNA, 18 bp, synthetic construct
  misc_feature 1..18: note = Synthetic oligonucleotide
  source 1..18: mol_type = other DNA
aagtacatttatatcaca

SEQ ID NO: 82 - DNA, 20 bp, synthetic construct
  misc_feature 1..20: note = Synthetic oligonucleotide
  source 1..20: mol_type = other DNA
catttattgtaaagcacaaa

SEQ ID NO: 83 - DNA, 18 bp, synthetic construct
  misc_feature 1..18: note = Synthetic oligonucleotide
  source 1..18: mol_type = other DNA
atcatttattgtaaagca

SEQ ID NO: 84 - RNA, 18 nt, synthetic construct
  misc_feature 1..18: note = siRNA sense
  source 1..18: mol_type = other RNA
gcacttcgcttcacctct

SEQ ID NO: 85 - DNA, 21 bp, synthetic construct
  misc_feature 1..21: note = Primer
  source 1..21: mol_type = other DNA
cgtctgtgccttctcatctgc

SEQ ID NO: 86 - DNA, 20 bp, synthetic construct
  misc_feature 1..20: note = primer
  source 1..20: mol_type = other DNA
gcacagcttggaggcttgaa

SEQ ID NO: 87 - DNA, 20 bp, synthetic construct
  misc_feature 1..20: note = primer
  source 1..20: mol_type = other DNA
ccgtctgaactatcctgccc

SEQ ID NO: 88 - DNA, 20 bp, synthetic construct
  misc_feature 1..20: note = primer
  source 1..20: mol_type = other DNA
gccgtagtcggtgtactcgt

SEQ ID NO: 89 - DNA, 43 bp, synthetic construct
  misc_feature 1..43: note = primer
  source 1..43: mol_type = other DNA
taatacgactcactatagggtttttcacctctgcctaatcatc

SEQ ID NO: 90 - DNA, 43 bp, synthetic construct; identical to SEQ ID NO: 89
  misc_feature 1..43: note = primer
  source 1..43: mol_type = other DNA

(19) United States
(12) Patent Application Publication
(10) Pub. No.: US 2024/0150758 A1
(43) Pub. Date: May 9, 2024

(54) USE OF FUBP1 INHIBITORS FOR TREATING HEPATITIS B VIRUS INFECTION

(71) Applicants: Hoffmann-La Roche, Inc., Little Falls, NJ (US); CENTRE LEON BERARD, Lyon (FR); UNIVERSITE CLAUDE BERNARD LYON I, Villeurbanne (FR); CENTRE NATIONAL DE LA RECHERCHE SCIENTIFIQUE, Paris (FR); INSERM - INSTITUT NATIONAL DE LA SANTÉ ET DE LA RECHERCHE MÉDICALE, Paris (FR)

(72) Inventors: Souphalone LUANGSAY, Basel (CH); Barbara TESTONI, Lyon (FR); Fabien ZOULIM, Lyon (FR); Soren OTTOSEN, Horsholm (DK); Lykke PEDERSEN, Horsholm (DK)

(21) Appl. No.: 18/340,054

(22) Filed: Jun. 23, 2023

Related U.S. Application Data
(62) Division of application No. 17/063,610, filed on Oct. 5, 2020, now Pat. No. 11,732,262.
Application No. 17/063,610 is a continuation of application No. PCT/EP2019/058664, filed on Apr. 5, 2019.

(30) Foreign Application Priority Data
Apr. 5, 2018 (EP) 18165897.2

(51) Int. Cl. C12N 15/113; A61K 31/4745; A61K 31/496; A61K 31/7125; A61P 31/20

(52) U.S. Cl. CPC C12N 15/113; A61K 31/4745; A61K 31/496; A61K 31/7125; A61P 31/20

(57) ABSTRACT

The present invention relates to a FUBPI inhibitor for use in treatment of an HBV infection, in particular a chronic HBV infection. The invention in particular relates to the use of FUBPI inhibitors for destabilizing cccDNA, such as HBV cccDNA. The invention also relates to nucleic acid molecules, such as oligonucleotides including siRNA, shRNA and antisense oligonucleotides, which are complementary to FUBPI and capable of reducing a FUBPI mRNA. Also comprised in the present invention is a pharmaceutical composition and its use in the treatment and/or prevention of a HBV infection.

### FIELD OF THE INVENTION

The present invention relates to Far Upstream Element-Binding Protein 1 (FUBP1) inhibitors for use in treating and/or preventing a hepatitis B virus (HBV) infection, in particular a chronic HBV infection. The invention in particular relates to the use of FUBP1 inhibitors for destabilizing cccDNA, such as HBV cccDNA. The invention also relates to nucleic acid molecules, such as oligonucleotides including siRNA, shRNA and antisense oligonucleotides, which are complementary to FUBP1 and capable of reducing a FUBP1 target nucleic acid, such as mRNA. Also comprised in the present invention is a pharmaceutical composition and its use in the treatment and/or prevention of a HBV infection.

### BACKGROUND

Far Upstream Element-Binding Protein 1 (FUBP1 or FBP1) is a single stranded DNA-binding protein that binds to multiple DNA elements. This protein is also thought to bind RNA and contains 3′-5′ helicase activity with in vitro activity on both DNA-DNA and RNA-RNA duplexes. FUBP1 is known to activate the transcription of the proto-oncogene c-myc by binding to far upstream element (FUSE) located upstream of c-myc in undifferentiated cells. The protein is primarily present in the nucleus of the cell. Upregulation of FUBP1 has been observed in many types of cancers. Furthermore, FUBP1 can bind to and mediate replication of RNA from Hepatitis C virus and Enterovirus (Zhang and Chen 2013 Oncogene vol 32 p. 2907-2916).

FUBP1 has also been identified in Hepatocellular carcinoma (HCC) where it has been suggested to be involved in HCC tumorigenesis (Ramdzan et al 2008 Proteomics Vol 8 p. 5086-5096) and that FUBP1 is required for HCC tumour growth as illustrated using lentivirus expressed shRNA targeting FUBP1 (Rabenhorst et al 2009 Hepatology vol 50 p 1121-1129).

It has been demonstrated that knock down of FUBP1 with lentivirus expressed shRNA's enhances treatment response in ovarian cancer (Zhang et al 2017 Oncology Letters Vol 14 p. 5819-5824).

WO 2004/027061 disclose a screening method which involves the step of analyzing whether or not a test substance inhibits FBP and a medicinal composition for treating a proliferative disease which contains as the active ingredient(s) a substance inhibiting FBP.

Some small molecules inhibiting FUBP1 have been identified, all with the purpose of treating cancer (Huth et al 2004 J Med. Chen Vol 47 p. 4851-4857; Hauck et al 2016 Bioorganic & Medicinal Chemistry Vol 24 p. 5717-5729 Hosseini et al 2017 Biochemical Pharmacology Vol 146 p. 53-62 and Xiong et al 2016 Int J Onc vol 49 p 623). WO2004/017940 describes lipid based formulations of SN-38, it claims treatment of viral infection, in particular HIV, there is however no example supporting this.

Poly(U) Binding Splicing Factor 60 (PUF60) is a potentially regulator of both transcriptional and post-transcriptional steps of HBV pregenome expression. PUF60 is known to form a complex with FUBP1 in relation to c-myc repression. FUBP1 does, however, not participate in the PUF60 dependent regulation of HBV pregenome expression (Sun et al 2017 Scientific Reports 7:12874).

To our knowledge FUBP1 has never been shown to bind cccDNA or associated with cccDNA stability, nor has molecules inhibiting FUBP1 ever been suggested as cccDNA destabilizers or for the treatment of HBV infection.

HBV infection remains a major health problem worldwide which concerns an estimated 350 million chronic carriers. Approximately 25% of carriers die from chronic hepatitis, cirrhosis, or liver cancer. Hepatitis B virus is the second most significant carcinogen behind tobacco, causing from 60% to 80% of all primary liver cancer. HBV is 100 times more contagious than HIV.

The hepatitis B virus (HBV) is an enveloped, partially double-stranded DNA virus. The compact 3.2 kb HBV genome consists of four overlapping open reading frames (ORF), which encode for the core, polymerase (Pol), envelope and X-proteins. The Pol ORF is the longest and the envelope ORF is located within it, while the X and core ORFs overlap with the Pol ORF. The lifecycle of HBV has two main events: 1) generation of closed circular DNA (cccDNA) from relaxed circular (RC DNA), and 2) reverse transcription of pregenomic RNA (pgRNA) to produce RC DNA.

HBsAg quantification is a significant biomarker for prognosis and treatment response in chronic hepatitis B. However the achievement of HBsAg loss and seroconversion (functional cure) is rarely observed in chronically infected patients. Hepatitis B e-antigen (also called HBV envelope antigen or HBeAg) is a viral protein that is secreted by hepatitis B infected cells. HBeAg is associated with chronic hepatitis B infections and is used as a marker of active viral disease and a patient's degree of infectiousness.

Accordingly, reducing secretion of HBeAg in addition to secretion of HBsAg would lead to an improved inhibition of development of a chronic HBV infection as compared to the inhibition of secretion of HBsAg alone.

Current therapy such as Nucleos(t)ide analogues are molecules that inhibit HBV DNA synthesis but are not directed at reducing HBsAg level. Most therapies currently under development aim to reach a functional cure, with a durable HBsAg loss±anti-HBs seroconversion, undetectable serum DNA, and cccDNA in a transcriptionally inactive state, but do not address cccDNA persistence. These approaches do not therefore lead to a complete cure of HBV infection which is defined as durable HBV DNA and HBsAg loss with cccDNA elimination. The persistence of cccDNA in infected hepatocytes is the main barrier for eradicating the virus in CHB patients, and there is an urgent need to develop new therapies for the HBV complete cure that eliminates cccDNA.

### OBJECTIVE OF THE INVENTION

The present invention shows that there is a correlation between the inhibition of FUBP1 and reduction of cccDNA in an HBV infected cell, which is relevant in the treatment of HBV infected individuals. An objective of the present invention is to identify FUBP1 inhibitors which reduce cccDNA in an HBV infected cell. Such FUBP1 inhibitors can be used in the treatment of HBV infection.

The present invention further identifies novel nucleic acid molecules, which are capable of inhibiting the expression of FUBP1 in vivo and in vitro.

### BRIEF DESCRIPTION OF THE FIGURES

FIG. 1: Illustrates exemplary antisense oligonucleotide conjugates, where the oligonucleotide either is represented as a wavy line (A-D) or as “oligonucleotide” (E-H) or as T₂(I) and the asialoglycoprotein receptor targeting conjugate moieties are trivalent N-acetylgalactosamine moieties. Compounds A to D comprise a di-lysine brancher molecule, a PEG3 spacer and three terminal GalNAc carbohydrate moieties. In compound A and B the oligonucleotide is attached directly to the asialoglycoprotein receptor targeting conjugate moiety without a linker. In compound C and D the oligonucleotide is attached to the asialoglycoprotein receptor targeting conjugate moiety via a C6 linker. Compounds E-I comprise a commercially available trebler brancher molecule and spacers of varying length and structure and three terminal GalNAc carbohydrate moieties.

FIG. 2: Structural formula of the trivalent GalNAc cluster (GN2). GN2 is useful as conjugation moiety in the present invention. The wavy line illustrates the site of conjugation of the cluster to e.g. a C6 amino linker or directly to the oligonucleotide.

FIG. 3: Southern blot from HBV infected PPH cells treated either with control (ctr) or the pool of FUBP1 targeting siRNA's (Pool) or HBV siRNA (HBV).

### SUMMARY OF THE INVENTION

One aspect of the present invention relates to a FUBP1 inhibitor for use in the treatment and/or prevention of Hepatitis B virus infection. In particular, a FUBP1 inhibitor capable of reducing cccDNA and/or pre-genomic RNA (pgRNA) is useful. In the event that the FUBP1 inhibitor is a small molecule it is advantageous if it interacts with the DNA binding domain of FUBP1 protein, and prevents or reduces FUBP1 binding to cccDNA. The FUBP1 inhibitor can also be a nucleic acid molecule of 12 to 60 nucleotides in length which is capable of reducing FUBP1 mRNA.

A further aspect of the present invention relates to nucleic acid molecules that inhibit expression and/or activity of FUBP1. In particular, a nucleic acid molecule of 12 to 60 nucleotides in length which comprises or consists of a contiguous nucleotide sequence of 12 to 30 nucleotides in length wherein the contiguous nucleotide sequence is at least 95% complementary to a mammalian FUBP1 target nucleic acid. Such nucleic acid molecules can be selected from single stranded antisense, siRNA and chemically produced shRNA molecules (not relying on cell based expression from plasmids or viruses).

A further aspect of the present invention relates to single stranded antisense oligonucleotides or siRNA that inhibit expression and/or activity of FUBP1. In particular, modified antisense oligonucleotides or modified siRNA comprising one or more 2′ sugar modified nucleoside and one or more phosphorthioate linkage that reduce FUBP1 mRNA are of advantageous.

Further aspects of the invention are conjugates of nucleic acid molecules of the invention and pharmaceutical compositions comprising the molecules of the invention. In particular conjugates targeting the liver are of interest, such as GalNAc clusters.

### Definitions

### Nucleic Acid Molecule

The term “nucleic acid molecule” or “therapeutic nucleic acid molecule” as used herein is defined as it is generally understood by the skilled person, as a molecule comprising two or more covalently linked nucleosides (i.e. a nucleotide sequence). The nucleic acid molecule(s) referred to in the method of the invention are generally therapeutic oligonucleotides below 50 nucleotides in length. The nucleic acid molecules may be or comprise an antisense oligonucleotide, or may be another oligomeric nucleic acid molecule, such as a CRISPR RNA, a siRNA, shRNA, an aptamer, or a ribozyme. Therapeutic nucleic acid molecules are commonly made in the laboratory by solid-phase chemical synthesis followed by purification and isolation. shRNA's are however often delivered to cells using lentiviral vectors (see for example Soan and Yang 2010 N Am J Med Sci 2(12): 598) which are then transcribed to produce the single stranded RNA that will form a stem loop (hairpin) RNA structure that is capable of interacting with the RNA interference machinery (including the RNA-induced silencing complex (RISC)). When referring to a sequence of the nucleic acid molecule, reference is made to the sequence or order of nucleobase moieties, or modifications thereof, of the covalently linked nucleotides or nucleosides. The nucleic acid molecule of the invention is man-made, and is chemically synthesized, and is typically purified or isolated. In some embodiments the nucleic acid molecule of the invention is not a shRNA transcribed from a vector upon entry into the target cell. The nucleic acid molecule of the invention may comprise one or more modified nucleosides or nucleotides.

In some embodiments, the nucleic acid molecule of the invention comprises or consists of 12 to 60 nucleotides in length, such as from 13 to 50, such as from 14 to 40, such as from 15 to 30, such as from 16 to 22, such as from 16 to 18 or 15 to 17 contiguous nucleotides in length.

In some embodiments, the nucleic acid molecule or contiguous nucleotide sequence thereof comprises or consists of 24 or less nucleotides, such as 22, such as 20 or less nucleotides, such as 18 or less nucleotides, such as 14, 15, 16 or 17 nucleotides. It is to be understood that any range given herein includes the range endpoints. Accordingly, if a nucleic acid molecule is said to include from 12 to 30 nucleotides, both 12 and 30 nucleotides are included.

In some embodiments, the contiguous nucleotide sequence comprises or consists of 12, 13, 14, 15, 16, 17, 18, 19, 20, 21 or 22 contiguous nucleotides in length

The nucleic acid molecule(s) are for modulating the expression of a target nucleic acid in a mammal. In some embodiments the nucleic acid molecules, such as for siRNAs, shRNAs and antisense oligonucleotides, are typically for inhibiting the expression of a target nucleic acid(s).

In one embodiment of the invention the nucleic acid molecule is selected from a RNAi agent, such as a siRNA or shRNA. In another embodiment the nucleic acid molecule is a single stranded antisense oligonucleotide, such as a high affinity modified antisense oligonucleotide interacting with RNaseH.

In some embodiments the nucleic acid molecule comprises phosphorothioate internucleoside linkages.

In some embodiments the nucleic acid molecule may be conjugated to non-nucleosidic moieties (conjugate moieties).

A library of nucleic acid molecules is to be understood as a collection of variant nucleic acid molecules. The purpose of the library of nucleic acid molecules can vary. In some embodiments, the library of nucleic acid molecules is composed of oligonucleotides with overlapping nucleobase sequence targeting one or more mammalian FUBP1 target nucleic acids with the purpose of identifying the most potent sequence within the library of nucleic acid molecules. In some embodiments, the library of nucleic acid molecules is a library of nucleic acid molecule design variants (child nucleic acid molecules) of a parent or ancestral nucleic acid molecule, wherein the nucleic acid molecule design variants retaining the core nucleobase sequence of the parent nucleic acid molecule.

### Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides, such as 2′ sugar modified nucleosides.

### Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide

Advantageously, the single stranded antisense oligonucleotide of the invention does not contain RNA nucleosides, since this will decrease nuclease resistance.

Advantageously, the antisense oligonucleotide of the invention comprises one or more modified nucleosides or nucleotides, such as 2′ sugar modified nucleosides. Furthermore, it is advantageous that the nucleosides which are not modified are DNA nucleosides.

### RNAi

Herein, the term “RNA interference (RNAi) molecule” refers to short double-stranded RNA molecule capable of inducing RNA-dependent gene silencing via the RNA-induced silencing complex (RISC) in a cell's cytoplasm, where they interact with the catalytic RISC component argonaute. One type of RNAi molecule is a small interfering RNA (siRNA), which is a double-stranded RNA molecule composed of two complementary oligonucleotides, where the binding of one strand to complementary mRNA after transcription, leads to its degradation and loss of translation. A small hairpin RNA (shRNA) is a single stranded RNA molecule that forms a stem loop (hairpin) structure which upon expression is able to reduce mRNA via the DICER and RNA reducing silencing complex (RISC). RNAi molecules can be designed based on the sequence of the gene of interest (target nucleic acid). Corresponding RNAi can then be synthesized chemically or by in vitro transcription, or expressed from a vector or PCR product.

shRNA molecules are generally between 40 and 70 nucleotides in length, such as between 45 and 65 nucleotides in length, such as 50 and 60 nucleotides in length, and interacts with the endonuclease known as Dicer which is believed to processes dsRNA into 19-23 base pair short interfering RNAs with characteristic two base 3′ overhangs which are then incorporated into an RNA-induced silencing complex (RISC). siRNA molecules are double stranded, with each strand being between18 and 35 nucleotides in length, such as 20 to 30 nucleotides in length, such as 22 to 27 nucleotides in length. siRNA's are often designed with a two base 3′ overhang to resemble the product produced by Dicer, which forms the RISC substrate. Effective extended forms of Dicer substrates have been described in U.S. Pat. No. 8,349,809 and U.S. Pat. No. 8,513,207, hereby incorporated by reference. Upon binding to the appropriate target mRNA, one or more endonucleases within the RISC cleave the target to induce silencing. RNAi olignucleotides may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA.

In some embodiments RNAi nucleic acid molecules comprise one or more phosphorothioate internucleoside linkages. In RNAi molecules phosphorothioate internucleoside linkages may reduce or the nuclease cleavage in RICS it is therefore advantageous that not al internucleoside linkages are modified. Phosphorothioate internucleoside linkages can advantageously be place in the 3′ and/or 5′ end of the RNAi nucleic acid molecule, in particular in the of the part of the molecule that is not complementary to the target nucleic acid (e.g. the sense stand or passenger strand in an siRNA molecule). The region of the RNAi molecule that is complementary to the target nucleic acid (e.g. the antisense or guide strand in an siRNA molecule) may however also be modified in the first 2 to 3 internucleoside linkages in the 3′ and/or 5′ terminal.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence and may optionally comprise further nucleotide(s), for example a nucleotide linker region which may be used to attach a functional group to the contiguous nucleotide sequence. The nucleotide linker region may or may not be complementary to the target nucleic acid.

### Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprises a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Internucleoside Linkage

The term “modified internucleoside linkage” is defined as generally understood by the skilled person as linkages other than phosphodiester (PO) linkages, that covalently couples two nucleosides together. The nucleic acid molecules of the invention may therefore comprise modified internucleoside linkages. In some embodiments, the modified internucleoside linkage increases the nuclease resistance of the nucleic acid molecules of the invention compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing nucleic acid molecules, single stranded antisense as well as double stranded siRNA and shRNA molecules for in vivo use, and may serve to protect against nuclease cleavage at regions of DNA or RNA nucleosides in nucleic acid molecules of the invention, for example within the gap region of a gapmer oligonucleotide, as well as in regions of modified nucleosides.

In an embodiment, the nucleic acid molecule, e.g. antisense oligonucleotide, shRNA or siRNA, comprises one or more internucleoside linkages modified from the natural phosphodiester, such as one or more modified internucleoside linkages, that is for example more resistant to nuclease attack. Nuclease resistance may be determined by incubating the oligonucleotide in blood serum or by using a nuclease resistance assay (e.g. snake venom phosphodiesterase (SVPD), both are well known in the art. Internucleoside linkages which are capable of enhancing the nuclease resistance of an oligonucleotide are referred to as nuclease resistant internucleoside linkages. In some embodiments at least 50% of the internucleoside linkages in the antisense oligonucleotide, or contiguous nucleotide sequence thereof, are modified, such as at least 60%, such as at least 70%, such as at least 75%, such as at least 80% or such as at least 90% of the internucleoside linkages in the oligonucleotide, or contiguous nucleotide sequence thereof, are modified. In some embodiments all of the internucleoside linkages of the oligonucleotide, or contiguous nucleotide sequence thereof, are modified. It will be recognized that, in some embodiments the nucleosides which link the oligonucleotide of the invention to a non-nucleotide functional group, such as a conjugate, may be phosphodiester. In some embodiments all of the internucleoside linkages of the oligonucleotide, or contiguous nucleotide sequence thereof, are nuclease resistant internucleoside linkages.

In some embodiments the internucleoside linkage comprises sulphur (S), such as a phosphorothioate internucleoside linkage. With the nucleic acid molecules of the invention it is advantageous to use phosphorothioate internucleoside linkages, in particular with single stranded antisense oligonucleotides.

Phosphorothioate internucleoside linkages are particularly useful due to nuclease resistance, beneficial pharmacokinetics and ease of manufacture. In some embodiments at least 20% of the internucleoside linkages in the nucleic acid molecule, or contiguous nucleotide sequence thereof, are phosphorothioate, such as at least 30%. For single stranded antisense oligonucleotides it is advantageous id at least 60% of the internucleoside linkages, such as at least 70%, such as at least 75%, such as at least 80% or such as at least 90% of the internucleoside linkages in the oligonucleotide, or contiguous nucleotide sequence thereof, are phosphorothioate. In some embodiments all of the internucleoside linkages of the antisense oligonucleotide, or contiguous nucleotide sequence thereof, are phosphorothioate. Nuclease resistant linkages, such as phosphorthioate linkages, are particularly useful in antisense oligonucleotide regions capable of recruiting nuclease when forming a duplex with the target nucleic acid, such as region G for gapmers. In an antisense gapmer oligonucleotide it is advantageous if the phosphodiester linkages, if present, are not located between contiguous DNA nucleosides in the gap region G. Advantageously, all the internucleoside linkages of the contiguous nucleotide sequence of the antisense oligonucleotide are phosphorothioate.

It is recognized that, as disclosed in EP 2 742 135, antisense oligonucleotides may comprise other internucleoside linkages (other than phosphodiester and phosphorothioate), for example alkyl phosphonate/methyl phosphonate internucleoside, which according to EP 2 742 135 may for example be tolerated in an otherwise DNA phosphorothioate the gap region.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Modified Oligonucleotide

The term modified oligonucleotide or modified nucleic acid molecule describes an oligonucleotide or nucleic acid molecule comprising one or more sugar-modified nucleosides and/or modified internucleoside linkages. The term “chimeric” is a term that has been used in the literature to describe oligonucleotides or nucleic acid molecules with modified nucleosides, in particular gapmer oligonucleotides.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison, a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

The following is an example of an oligonucleotide that is fully complementary to the target nucleic acid.

The following is an example of an oligonucleotide motif (SEQ ID NO: 33) that is fully complementary to the target nucleic acid (SEQ ID NO: 22).

| (SEQ ID NO: 22) | |
|---|---|
| | 5′ gagaaguucggaaugagua 3′ |
| (SEQ ID NO: 33) | |
| | 3′ cttcaagccttactc 5′ |

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Hybridization

The term “hybridizing” or “hybridizes” as used herein is to be understood as two nucleic acid strands (e.g. an antisense oligonucleotide or siRNA guide strand and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

### Target Nucleic Acid

According to the present invention, the target nucleic acid is a nucleic acid which encodes mammalian FUBP1 and may for example be a gene, a RNA, a mRNA, and pre-mRNA, a mature mRNA or a cDNA sequence. The target may therefore be referred to as a FUBP1 target nucleic acid.

The therapeutic nucleic acid molecules of the invention may for example target exon regions of a mammalian FUBP1 (in particular siRNA and shRNA, but also antisense oligonucleotides), or may for example target any intron region in the FUBP1 pre-mRNA (in particular antisense oligonucleotides). Table 1 lists predicted exon and intron regions of SEQ ID NO: 1.

TABLE 1
Exon and intron regions in the human FUBP1 pre-mRNA.

| | |
|---|---|
| Exonic regions in the | Intronic regions in the |
| human FUBP1 premRNA | human FUBP1 premRNA |
| (SEQ ID NO 1) | (SEQ ID NO 1) |

| ID | start | end | ID | start | end |
|---|---|---|---|---|---|
| E1 | 19 | 226 | I1 | 227 | 9095 |
| E2 | 9096 | 9186 | I2 | 9187 | 10907 |
| E3 | 10908 | 10946 | I3 | 10947 | 11444 |
| E4 | 11445 | 11484 | I4 | 11485 | 12009 |
| E5 | 12010 | 12062 | I5 | 12063 | 12155 |
| E6 | 12156 | 12227 | I6 | 12228 | 12359 |
| E7 | 12360 | 12417 | I7 | 12418 | 13879 |
| E8 | 13880 | 14042 | I8 | 14043 | 14142 |
| E9 | 14143 | 14241 | I9 | 14242 | 14363 |
| E10 | 14364 | 14465 | I10 | 14466 | 14754 |
| E11 | 14755 | 14857 | I11 | 14858 | 14948 |
| E12 | 14949 | 15049 | I12 | 15050 | 15395 |
| E13 | 15396 | 15537 | I13 | 15538 | 16180 |
| E14 | 16181 | 16341 | I14 | 16342 | 18615 |
| E15 | 18616 | 18767 | I15 | 18768 | 18847 |
| E16 | 18848 | 18927 | I16 | 18928 | 22410 |
| E17 | 22411 | 22539 | I17 | 22540 | 23781 |
| E18 | 23782 | 23856 | I18 | 23857 | 29810 |
| E19 | 29811 | 29956 | I19 | 29957 | 30196 |
| E20 | 30197 | 30706 | | | |

Suitably, the target nucleic acid encodes a FUBP1 protein, in particular mammalian FUBP1, such as human FUBP1(See for example Tables 2 and 3) which provides the genomic sequence, the mature mRNA and pre-mRNA sequences for human, monkey and mouse FUBP1).

In some embodiments, the target nucleic acid is selected from the group consisting of SEQ ID NO: 2, 3, 4, 6, 7, 8, 10, 11, 12, 13, 14, 15, 16, 17, 18, 19 and/or 20, or naturally occurring variants thereof (e.g. sequences encoding a mammalian FUBP1).

In some embodiments, the target nucleic acid is selected from the group consisting of SEQ ID NO: 1, 5 and/or 9, or naturally occurring variants thereof (e.g. sequences encoding a mammalian FUBP1).

In some embodiments, the target nucleic acid is selected from the group consisting of SEQ ID NO: 1 and 5, or naturally occurring variants thereof (e.g. sequences encoding a mammalian FUBP1).

In some embodiments, the target nucleic acid is selected from the group consisting of SEQ ID NO: 1, to 8, or naturally occurring variants thereof (e.g. sequences encoding a mammalian FUBP1).

TABLE 2
Genome and assembly information for FUBP1 across species.

| | | | Genomic coordinates | | | ensembl |
|---|---|---|---|---|---|---|
| Species | Chr. | Strand | Start | End | Assembly | gene_id |
| Human | 1 | Rv | 77944055 | 77979110 | GRCh38. | ENSG |
| | | | | | p10 | 00000162613 |
| Cyno | 1 | Fwd | 149243675 | 149283374 | Macaca_ | ENSMFAG |
| mon- | | | | | fascicu- | 00000031825 |
| key | | | | | laris_ | |
| | | | | | 5.0 | |
| Mouse | 3 | Fwd | 152210422 | 152236826 | GRCm38. | ENSMUSG |
| | | | | | p5 | 00000028034 |

Fwd = forward strand.
Rv = reverse strand.
The genome coordinates provide the pre-mRNA sequence (genomic sequence).

If employing the nucleic acid molecule of the invention in research or diagnostics the target nucleic acid may be a cDNA or a synthetic nucleic acid derived from DNA or RNA.

For in vivo or in vitro application, the therapeutic nucleic acid molecule of the invention is typically capable of inhibiting the expression of the FUBP1 target nucleic acid in a cell which is expressing the FUBP1 target nucleic acid. The contiguous sequence of nucleobases of the nucleic acid molecule of the invention is typically complementary to a conserved region of the FUBP1 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides. Further information on exemplary target nucleic acids is provided in table 3.

TABLE 3
Sequence details for FUBP1 across species.

| Species | RNA type | Length (nt) | SEQ ID NO |
|---|---|---|---|
| Human | Pre-mRNA | 305056 | 1 |
| Human | Mature mRNA | 696 | 2 |
| Human | Mature mRNA | 1968 | 3 |
| Human | Mature mRNA | 1935 | 3 |
| Cyno monkey | Pre-mRNA | 39750 | 5 |
| Cyno monkey | Mature mRNA | 1968 | 6 |
| Cyno monkey | Mature mRNA | 6825 | 7 |
| Cyno monkey | Mature mRNA | 1959 | 8 |
| Mouse | Pre-mRNA | 26405 | 9 |
| Mouse | Mature mRNA | 4525 | 10 |
| Mouse | Mature mRNA | 800 | 11 |
| Mouse | Mature mRNA | 2526 | 12 |
| Mouse | Mature mRNA | 809 | 13 |
| Mouse | Mature mRNA | 1040 | 14 |
| Mouse | Mature mRNA | 796 | 15 |
| Mouse | Mature mRNA | 585 | 16 |
| Mouse | Mature mRNA | 2374 | 17 |
| Mouse | Mature mRNA | 3163 | 18 |
| Mouse | Mature mRNA | 6523 | 19 |
| Mouse | Mature mRNA | 2552 | 20 |

Note SEQ ID NO 5 comprises regions of multiple NNNNs, where the sequencing has been unable to accurately refine the sequence, and a degenerate sequence is therefore included. For the avoidance of doubt the compounds of the invention are complementary to the actual target sequence and are not therefore degenerate compounds.

### Target Sequence

The term “target sequence” as used herein refers to a sequence of nucleotides present in the target nucleic acid which comprises the nucleobase sequence which is complementary to the oligonucleotide or nucleic acid molecule of the invention. In some embodiments, the target sequence consists of a region on the target nucleic acid with a nucleobase sequence that is complementary to the contiguous nucleotide sequence of the oligonucleotide of the invention (i.e. a sub-sequence). This region of the target nucleic acid may interchangeably be referred to as the target nucleotide sequence, target sequence or target region. In some embodiments the target sequence is longer than the complementary sequence of a single nucleic acid molecule, and may, for example represent a preferred region of the target nucleic acid which may be targeted by several nucleic acid molecules of the invention.

In some embodiments the target sequence is a sequence selected from the group consisting of a human FUBP1 mRNA exon, such as a FUBP1 human mRNA exon selected from the group consisting of e1, e2, e3, e4, e5, e6, e7, e8, e9, e10, e11, e12, 13, e14, e15, e16, e17, e18, e19 and e20 (see for example table 1 above).

In one embodiment the target sequence is a sequence selected from the group consisting of one or more of human FUBP1 mRNA exons selected from the group consisting of exon 9, 10, 12 and 20.

In some embodiments the target sequence is a sequence selected from the group consisting of a human FUBP1mRNA intron, such as a FUBP1 human mRNA intron selected from the group consisting of i1, i2, i3, 14, 15, 16, i7, 19, i10, i11, i12, 13, i14, 115, i16, i17, i18 and i19 (see for example table 1 above).

The nucleic acid molecule of the invention comprises a contiguous nucleotide sequence which is complementary to or hybridizes to a region on the target nucleic acid, such as a target sequence described herein.

The target nucleic acid sequence to which the therapeutic nucleic acid molecule is complementary or hybridizes to generally comprises a stretch of contiguous nucleobases of at least 10 nucleotides. The contiguous nucleotide sequence is between 12 to 70 nucleotides, such as 12-50, such as 13 to 30, such as 14 to 25, such as 15 to 20, such as 16 to 18 contiguous nucleotides.

### Target Cell

The term a “target cell” as used herein refers to a cell which is expressing the target nucleic acid. For the therapeutic use of the present invention it is advantageous if the target cell is infected with HBV. In some embodiments the target cell may be in vivo or in vitro. In some embodiments the target cell is a mammalian cell such as a rodent cell, such as a mouse cell or a rat cell, or a primate cell such as a monkey cell or a human cell.

In preferred embodiments the target cell expresses FUBP1 mRNA, such as the FUBP1pre-mRNA or FUBP1 mature mRNA. The poly A tail of FUBP1 mRNA is typically disregarded for nucleic acid molecule targeting.

### Naturally Occurring Variant

The term “naturally occurring variant” refers to variants of theFUBP1 gene or transcripts which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms, and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian FUBP1 target nucleic acid, such as a target nucleic acid selected form the group consisting of SEQ ID NO: 1, 5 or 9. In some embodiments the naturally occurring variants have at least 99% homology to the human FUBP1 target nucleic acid of SEQ ID NO: 1. In some embodiments the naturally occurring variants are known polymorphisms.

### Modulation of Expression

The term “modulation of expression” as used herein is to be understood as an overall term for a nucleic acid molecules ability to alter the amount of FUBP1 when compared to the amount of FUBP1 before administration of the nucleic acid molecule. Alternatively, modulation of expression may be determined by reference to a control experiment. It is generally understood that the control is an individual or target cell treated with a saline composition or an individual or target cell treated with a non-targeting or nucleic acid molecule (mock). It may however also be an individual treated with the standard of care.

One type of modulation is the ability of a nucleic acid molecule to inhibit, down-regulate, reduce, remove, stop, prevent, lessen, lower, avoid or terminate expression of FUBP1, e.g. by degradation of mRNA or blockage of transcription.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C. , more preferably between +1.5 to +10° C. and most preferably between+3 to +8° C. per modified nucleoside. Numerous high affinity modified nucleosides are known in the art and include for example, many 2′ sugar modified nucleosides, such as 2′ substituted nucleosides like Ome and MOE as well as 2′ to 4′ bridged nucleic acids such as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

### Sugar Modifications

The nucleic acid molecule of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of nucleic acid molecules, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the —OH groups naturally found in RNA or DNA nucleosides. Substituents may, for example be introduced at the 2′, 3′, 4′ or 5′ positions.

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

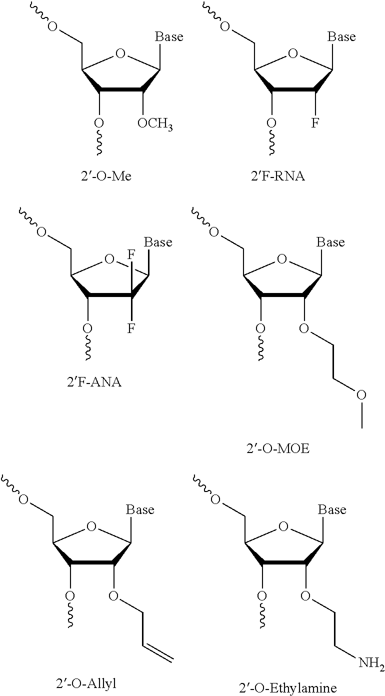

In relation to the present invention 2′ substituted does not include 2′ bridged molecules like LNA.

### Locked Nucleic Acid Nucleosides (LNA).

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

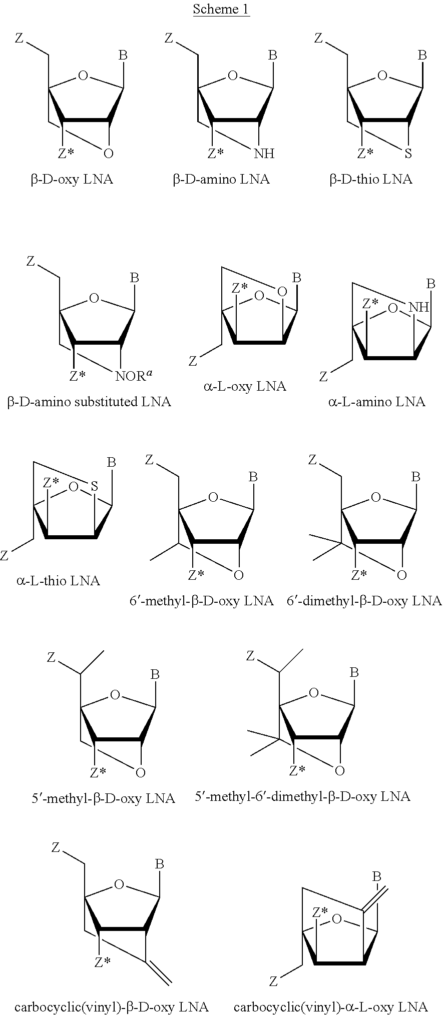

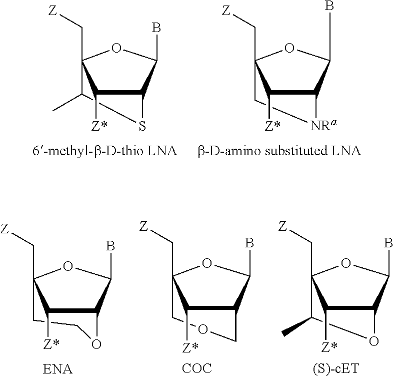

Particular LNA nucleosides are beta-D-oxy-LNA, 6′-methyl-beta-D-oxy LNA such as (S)-6′-methyl-beta-D-oxy-LNA (ScET) and ENA. A particularly advantageous LNA is beta-D-oxy-LNA.

### Nuclease Mediated Degradation

Nuclease mediated degradation refers to nucleic acid molecule capable of mediating degradation of a complementary nucleotide sequence when forming a duplex with such a sequence.

In some embodiments, the nucleic acid molecule may function via nuclease mediated degradation of the target nucleic acid. In particular, antisense oligonucleotides are capable of recruiting an endoribonuclease (RNase) which recognizes RNA/DNA hybridization and effects cleavage of the RNA nucleic acid. RNase H has proven to be an advantageous endoribonuclease. Examples of antisense oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers, headmers and tailmers.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland

### Gapmer

The nucleic acid molecule of the invention, or contiguous nucleotide sequence thereof may be a gapmer antisense oligonucleotide. The antisense gapmers are commonly used to inhibit a target nucleic acid via RNase H mediated degradation. In an embodiment of the invention the oligonucleotide of the invention is capable of recruiting RNase H.

A gapmer antisense oligonucleotide comprises at least three distinct structural regions a 5′-flank, a gap and a 3′-flank, F-G-F′ in the ‘5 ->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 17, such as 16 to 18 nucleosides. By way of example, the gapmer oligonucleotide of the present invention can be represented by the following formulae:

F₁₋₈-G₅₋₁₆-F′₁₋₈, such as

F₁₋₈-G₇₋₁₆-F′₂₋₈

with the proviso that the overall length of the gapmer regions F-G-F′ is at least 12, such as at least 14 nucleotides in length.

In an aspect of the invention the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and defines the 5′ and 3′ end of the F and F′ region, and G is a region between 6 and 16 nucleosides which are capable of recruiting RNaseH.

In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

Further gapmer designs are disclosed in WO2004/046160, WO2007/146511 and WO2008/113832, hereby incorporated by reference.

### Conjugate

The term conjugate as used herein refers to an oligonucleotide which is covalently linked to a non-nucleotide moiety (conjugate moiety or region C or third region).

Conjugation of the therapeutic nucleic acid molecule of the invention to one or more non-nucleotide moieties may improve the pharmacology of the nucleic acid molecule, e.g. by affecting the activity, cellular distribution, cellular uptake or stability of the nucleic acid molecule. In some embodiments the conjugate moiety, modify or enhance the pharmacokinetic properties of the nucleic acid molecule by improving cellular distribution, bioavailability, metabolism, excretion, permeability, and/or cellular uptake of the oligonucleotide. In particular, the conjugate may target the nucleic acid molecule to a specific organ, tissue or cell type and thereby enhance the effectiveness of the oligonucleotide in that organ, tissue or cell type. At the same time the conjugate may serve to reduce activity of the nucleic acid molecule in non-target cell types, tissues or organs, e.g. off target activity or activity in non-target cell types, tissues or organs. For siRNA nucleic acid molecules the conjugate moiety is most commonly covalently linked to the passenger strand of the siRNA, and for shRNA molecules the conjugate moiety would most commonly be linked to the end of the molecule which is furthest away from the contiguous nucleotide sequence of the shRNA. For antisense oligonucleotides the conjugate moiety can be covalently linked to any of the terminal ends, advantageously using a biocleavable linker such as a 2 to 5 phosphodiester linked DNA nucleosides.

WO 93/07883 and WO2013/033230 provides suitable conjugate moieties, which are hereby incorporated by reference. Further suitable conjugate moieties are those capable of binding to the asialoglycoprotein receptor (ASGPR). In particular tri-valent N-acetylgalactosamine conjugate moieties are suitable for binding to the ASGPR, see for example US 2009/02398, WO 2014/076196, WO 2014/207232 and WO 2014/179620 (hereby incorporated by reference).

Such conjugates serve to enhance uptake of the therapeutic nucleic acid molecule to the liver while reducing its presence in the kidney, thereby increasing the liver/kidney ratio of a conjugated nucleic acid molecule compared to the unconjugated version of the same nucleic acid molecule.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

### Conjugate Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect one region, e.g. a conjugate moiety to another region, e.g. an oligonucleotide (e.g. the termini of region A or C).

In some embodiments of the invention the conjugate or therapeutic nucleic acid molecule conjugate of the invention may optionally, comprise a linker region which is positioned between the oligonucleotide and the conjugate moiety. In some embodiments, the linker between the conjugate and oligonucleotide is physiologically labile linker, interchangeably termed biocleavable linker. The linker and the oligonucleotide is often attached via a phosphodiester linkage.

Biocleavable linkers (Region B) comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In some embodiments the physiologically labile linker (biocleavable) comprises between 1 and 10 linked nucleosides, such as 1, 2, 3, 4, 5, 6, 7, 8, 9 or 10 linked nucleosides, such as between 2 and 6 linked nucleosides, such as between 2 and 5 linked nucleosides, such as between 2 and 4 linked nucleosides, where at least two consecutive linkages are biocleavable, such as phosphodiester linkages, such as at least 3 or 4 or 5 consecutive phosphodiester linkages. Preferably the nucleosides are DNA or RNA.

In one embodiment the linker between the oligonucleotide and the conjugate moiety is a physiologically labile linker composed of 2 to 5 consecutive phosphodiester linked nucleosides comprising at least two consecutive phosphodiester linkages at the 5′ or 3′ terminal of the contiguous nucleotide sequence of the antisense oligonucleotide or siRNA guide strand.

In another embodiment the linker between the oligonucleotide and the conjugate moiety is a physiologically labile linker composed of 2 to 5 consecutive phosphodiester linked nucleosides comprising at least two consecutive phosphodiester linkages at the 5′ or 3′ terminal of the passenger strand of the siRNA,

In another embodiment the linker between the oligonucleotide and the conjugate moiety is a physiologically labile linker composed of 2 to 5 consecutive phosphodiester linked nucleosides comprising at least two consecutive phosphodiester linkages at the terminal end of the shRNA molecule furthest away from the contiguous nucleotide sequence of the shRNA.

In some embodiments the physiologically labile linker comprises or consist of a DNA dinucleotide with a sequence selected from the group consisting of AA, AT, AC, AG, TA, TT, TC, TG, CA, CT, CC, CG, GA, GT, GC, or GG, where there is a phosphodiester linkage between the two DNA nucleosides and at least one further phosphodiester at the 5′ or 3′ end of the dinucleotide linking either the oligonucleotide of the nucleic acid molecule to the dinucleotide or the conjugate moiety to the dinucleotide. In some embodiments the physiologically labile linker comprises or consist of a DNA trinucleotide of sequence AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TAG, TCA, TCT, TCC, TCG, TGA, TGT, TGC, TGG, CAA, CAT, CAC, CAG, CTA,

CTG, CTC, CTT, CCA, CCT, CCC, CCG, CGA, CGT, CGC, CGG, GAA, GAT, GAC, CAG, GTA, GTT, GTC, GTG, GCA, GCT, GCC, GCG, GGA, GGT, GGC, or GGG, where there is phosphodiester linkages between the DNA nucleosides and potentially a further phosphodiester at the 5′ or 3′ end of the trinucleotide. Phosphodiester containing biocleavable linkers are described in more detail in WO 2014/076195 (hereby incorporated by reference). In a conjugate compound with a biocleavable linker at least about 50% of the conjugate moiety is cleaved from the oligonucleotide, such as at least about 60% cleaved, such as at least about 70% cleaved, such as at least about 80% cleaved, such as at least about 85% cleaved, such as at least about 90% cleaved, such as at least about 95% of the conjugate moiety is cleaved from the oligonucleotide cleaved when compared against a standard.

Conjugates may also be linked to the oligonucleotide via non-biocleavable linkers, or in some embodiments the conjugate may comprise a non-cleavable linker which is covalently attached to the biocleavable linker. Linkers that are not necessarily biocleavable primarily serve to covalently connect a conjugate moiety to the oligonucleotide or biocleavable linker, and potentially generate some distance between the conjugate moiety and the oligonucleotide. Some example linkers (region Y) include 8-amino-3,6-dioxaoctanoic acid (ADO), succinimidyl 4-(N-maleimidomethyl)cyclohexane-I-carboxylate (SMCC), 6-aminohexanoic acid (AHEX or AHA), 6-aminohexyloxy, 4-aminobutyric acid, 4-aminocyclohexylcarboxylic acid, succinimidyl 4-(N-maleimidomethyl)cyclohexane-I-carboxy-(6-amido-caproate) (LCSMCC), succinimidyl m-maleimido-benzoylate (MBS), succinimidyl N-e-maleimido-caproylate (EMCS), succinimidyl 6-(beta-maleimido-propionamido) hexanoate (SMPH), succinimidyl N-(a-maleimido acetate) (AMAS), succinimidyl 4-(p-maleimidophenyl)butyrate (SMPB), beta-alanine (beta-ALA), phenylglycine (PHG), 4-aminocyclohexanoic acid (ACHC), beta-(cyclopropyl) alanine (beta-CYPR), amino dodecanoic acid (ADC), alylene diols, polyethylene glycols, amino acids, and the like. Non-cleavable linkers may also comprise a chain structure or an oligomer of repeating units such as ethylene glycol, amino acid units or amino alkyl groups. In some embodiments the linker (region Y) is an amino alkyl, such as a C₂-C₃₆ amino alkyl group, including, for example C₆ to C₁₂ amino alkyl groups. In some embodiments the linker (region Y) is a C₆ amino alkyl group (also termed a C6 linker). Conjugate linker groups may be routinely attached to an oligonucleotide via use of an amino modified oligonucleotide, and an activated ester group on the conjugate group. The linkage group between the amino alkyl and the oligonucleotide may for example be a phosphorothioate or a phosphodiester, or one of the other nucleoside linkage groups referred to herein. A conjugate compound of the present invention may be composed of the following regions C—B-A (Conjugate moiety-biocleavable linker-oligonucleotide/contiguous nucleotide sequence) or C—Y—B-A (conjugate moiety-non-cleavable linker-biocleavable linker-oligonucleotide/contiguous nucleotide sequence).

### Treatment

The terms “treatment”, “treating”, “treats” or the like as used herein generally means obtaining a desired pharmacological and/or physiological effect. This effect is therapeutic in terms of partially or completely curing a disease and/or adverse effect attributed to the disease. The term “treatment” as used herein covers any treatment of a disease in a subject and includes: (a) inhibiting the disease, i.e. arresting its development like the inhibition of increase of HBsAg and/or HBeAg; or (b) ameliorating (i.e. relieving) the disease, i.e. causing regression of the disease, like the repression of HBsAg and/or HBeAg production. Thus, a compound that ameliorates and/or inhibits a HBV infection is a compound that treats a HBV invention. Preferably, the term “treatment” as used herein relates to medical intervention of an already manifested disorder, like the treatment of an already defined and manifested HBV infection.

### Prevention

Herein the term “preventing”, “prevention” or “prevents” relates to a prophylactic treatment, i.e. to a measure or procedure the purpose of which is to prevent, rather than to cure a disease. Prevention means that a desired pharmacological and/or physiological effect is obtained that is prophylactic in terms of completely or partially preventing a disease or symptom thereof. Accordingly, herein “preventing a HBV infection” includes preventing a HBV infection from occurring in a subject, and preventing the occurrence of symptoms of a HBV infection. In the present invention in particular the prevention of HBV infection in children from HBV infected mothers are contemplated. Also contemplated is the prevention of an acute HBV infection turning into a chronic HBV infection.

### Patient

For the purposes of the present invention the “subject” (or “patient”) may be a vertebrate. In context of the present invention, the term “subject” includes both humans and other animals, particularly mammals, and other organisms. Thus, the herein provided means and methods are applicable to both human therapy and veterinary applications. Accordingly, herein the subject may be an animal such as a mouse, rat, hamster, rabbit, guinea pig, ferret, cat, dog, chicken, sheep, bovine species, horse, camel, or primate. Preferably, the subject is a mammal. More preferably the subject is human.

### HBV Infection

The term “hepatitis B virus infection” or “HBV infection” is commonly known in the art and refers to an infectious disease that is caused by the hepatitis B virus (HBV) and affects the liver. A HBV infection can be an acute or a chronic infection. Some infected persons have no symptoms during the initial infection and some develop a rapid onset of sickness with vomiting, yellowish skin, tiredness, dark urine and abdominal pain (“Hepatitis B Fact sheet N° 204”. who.int. July 2014. Retrieved 4 Nov. 2014). Often these symptoms last a few weeks and can result in death. It may take 30 to 180 days for symptoms to begin. In those who get infected around the time of birth 90% develop a chronic hepatitis B infection while less than 10% of those infected after the age of five do (“Hepatitis B FAQs for the Public—Transmission”, U.S. Centers for Disease Control and Prevention (CDC), retrieved 2011-11-29). Most of those with chronic disease have no symptoms; however, cirrhosis and liver cancer may eventually develop (Chang, 2007, Semin Fetal Neonatal Med, 12: 160-167). These complications result in the death of 15 to 25% of those with chronic disease (“Hepatitis B Fact sheet N° 204”. who.int. July 2014, retrieved 4 Nov. 2014). Herein, the term “HBV infection” includes the acute and chronic hepatitis B infection. The term “HBV infection” also includes the asymptotic stage of the initial infection, the symptomatic stages, as well as the asymptotic chronic stage of the HBV infection.

cccDNA cccDNA (covalently closed circular DNA) is a special DNA structure that arises during the propagation of some DNA viruses (Polyomaviridae) in the cell nucleus. cccDNA is a double-stranded DNA that originates in a linear form that is ligated by means of DNA ligase to a covalently closed ring. In most cases, transcription of viral DNA can occur from the circular form only. The cccDNA of viruses is also known as episomal DNA or occasionally as a minichromosome.

cccDNA is typical of Caulimoviridae and Hepadnaviridae, including the hepatitis B virus (HBV). The HBV genome forms a stable minichromosome, the covalently closed circular DNA (cccDNA), in the hepatocyte nucleus. The cccDNA is formed by conversion of capsid-associated relaxed circular DNA (rcDNA). HBV cccDNA formation involves a multi-step process that requires the cellular DNA repair machinery and relies on specific interactions with distinct cellular components that contribute to the completion of the positive strand DNA in rcDNA (Alweiss et al. 2017, Viruses, 9 (6): 156).

### Pharmaceutically Acceptable Salts

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

### Inhibitor

The term “inhibitor” is known in the art and relates to a compound/substance or composition capable of fully or partially preventing or reducing the physiologic function (i.e. the activity) of (a) specific protein(s) (e.g. of FUBP1). In the context of the present invention, an “inhibitor” of FUBP1 is capable of preventing or reducing the activity/function of FUBP1, respectively, by preventing or reducing the expression of the FUBP1 gene products. Thus, an inhibitor of FUBP1 may lead to a decreased expression level of FUBP1 (e.g. decreased level of FUBP1 mRNA, or of FUBP1protein) which is reflected in a decreased functionality (i.e. activity) of FUBP1, wherein said function comprises the poly-A polymerase function. An inhibitor of FUBP1, in the context of the present invention, accordingly, may also encompass transcriptional repressors of FUBP1 expression that are capable of reducing the level of FUBP1. Preferred inhibitors are nucleic acid molecules of the invention.

### Compound

Herein, the term “compound” means any molecule capable of inhibition FUBP1 expression or activity. Particular compounds of the invention are nucleic acid molecules, such as RNAi molecules or antisense oligonucleotides according to the invention or any conjugate comprising such a nucleic acid molecule. For example, herein the compound may be a nucleic acid molecule targeting FUBP1, in particular an antisense oligonucleotide or a siRNA.

### Composition

The term “composition” may also be used to describe a nucleic acid molecule compound or another FUBP1 inhibitor. A nucleic acid molecule composition has less than 20% impurities, preferably less than 15% or 10% impurities, more preferably less than 9, 8, 7 or 6% impurities, most preferably less than 5% impurities. The impurities are typically nucleic acid molecules which are one or two nucleotides shorter (n−1 or n−2) than the primary nucleic acid molecule component.

The present invention is further described by reference to the non-limiting figures and examples.

### DETAILED DESCRIPTION OF THE INVENTION

Overexpression of and mutations in FUBP1 has been known to be associated with cancers for many years. In particular, strong overexpression of FUBP1 in human hepatocellular carcinoma

(HCC) supports tumour growth and correlates with poor patient prognosis. HBV cccDNA in infected hepatocytes is responsible for persistent chronic infection and reactivation, being the template for all viral subgenomic transcripts and pre-genomic RNA (pgRNA) to ensure both newly synthesized viral progeny and cccDNA pool replenishment via intracellular nucleocapsid recycling. In the context of the present invention it was for the first time shown that FUBP1 is associated with cccDNA stability. This knowledge allows for the opportunity to destabilize cccDNA in HBV infected subjects which in turn opens the opportunity for a complete cure of chronically infected HBV patients. The role of FUBP1 in HCC and cccDNA stability is expected to be different and independent of each other.

One aspect of the present invention is a FUBP1 inhibitor for use in the treatment and/or prevention of Hepatitis B virus (HBV) infection, in particular a chronic HBV infection.

An embodiment of the invention is a FUBP1 inhibitor which is capable of reducing cccDNA and pgRNA in an infected cell, such as an HBV infected cell.

In a further embodiment, the FUBP1 inhibitor is capable of reducing HBsAg and/or HBeAg in vivo in an HBV infected individual.

### FUBP1 Inhibitors for Use in Treatment of HBV

Without being bound by theory, it is believed that FUBP1 is involved in the stabilization of the cccDNA in the cell nucleus, and by preventing the binding of FUBP1 to DNA, in particular cccDNA, the cccDNA is destabilised and becomes prone to degradation. One embodiment of the invention is therefore a FUBP1 inhibitor which interacts with the DNA binding domain of FUBP1 protein, and prevents or reduces binding to cccDNA.

Small molecules inhibiting FUBP1 have been identified in relation to FUBP1′s role in cancer, where the small molecule inhibits the DNA binding activity of FUBP1, in particular the binding to the FUSE element on a single stranded DNA. In the present invention FUBP1 inhibitors are envisioned as useful in treating HBV. In particular targeting of such small molecule compounds, e.g. via conjugation or formulation, to the liver may be beneficial in the treatment of HBV.

Huth et al 2004 J Med. Chem Vol 47 p. 4851-4857 discloses a series of benzoyl anthranilic acid compounds capable of binding to a four tandem K homology (KH) repeat of FUBP1. All the compounds disclosed in Huth et al 2004 are hereby incorporated by reference. In particular the compounds of formula I, II or III shown below were found to be efficient in inhibiting FUBP1 DNA binding activity.

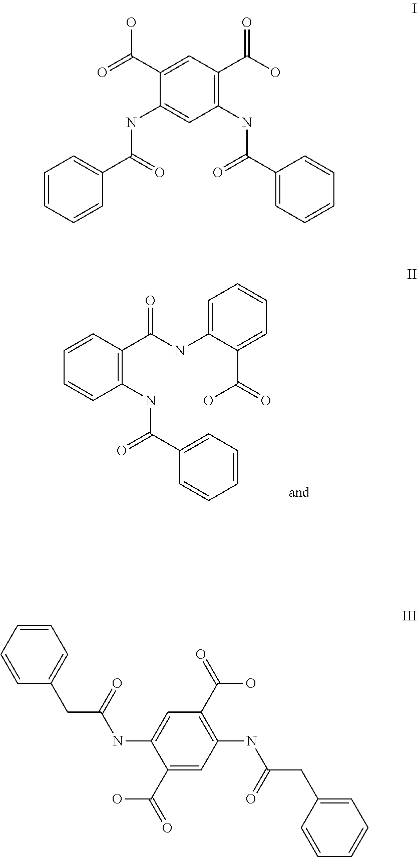 [SMILES: O=C(Nc1cc(NC(=O)c2ccccc2)c(C(=O)O)cc1C(=O)O)c1ccccc1 ; O=C(Nc1ccccc1C(=O)Nc1ccccc1C(=O)O)c1ccccc1 ; O=C(Cc1ccccc1)Nc1cc(C(=O)O)c(NC(=O)Cc2ccccc2)cc1C(=O)O]

One embodiment of the present invention is a compound of formula I, II or III for use in treatment and/or prevention of Hepatitis B virus (HBV) infection.

Hauck et al 2016 Bioorganic & Medicinal Chemistry Vol 24 p. 5717-5729 describes an additional series of compounds with high FUBP1 inhibitory potential (see table 2, hereby incorporated by reference). In particular, the following compounds of formula IV were effective in inhibiting FUBP1 activity

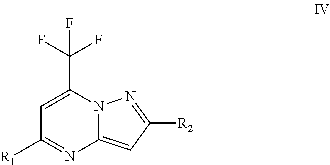

wherein R1 is selected from

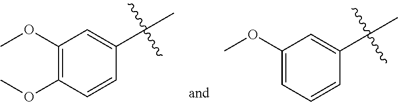

and R2 is selected from

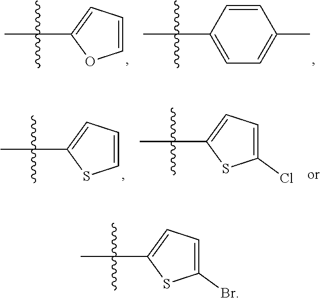

Specifically the compounds of formula V, VI and VII were shown to have IC50 values below 15 μM.

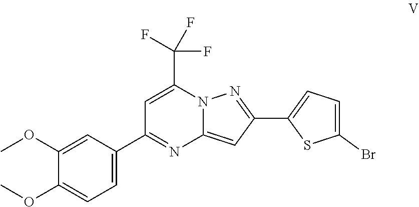 [SMILES: COc1ccc(-c2cc(C(F)(F)F)n3nc(-c4ccc(Br)s4)cc3n2)cc1OC]

### 2-(5-Bromothiophen-2-yl)-5-(3,4-dimethoxyphenyl)-7-(trifluoromethyl)pyrazolo[1,5-a]pyrimidine

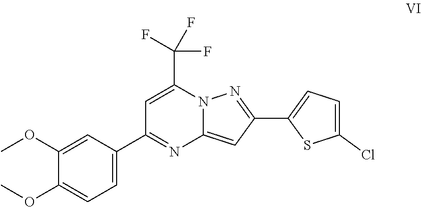 [SMILES: COc1ccc(-c2cc(C(F)(F)F)n3nc(-c4ccc(Cl)s4)cc3n2)cc1OC]

### 2-(5-Chlorothiophen-2-yl)-5-(3,4-dimethoxyphenyl)-7-(trifluoromethyl)pyrazolo[1,5-a]pyrimidine

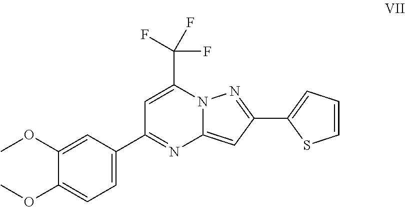 [SMILES: COc1ccc(-c2cc(C(F)(F)F)n3nc(-c4cccs4)cc3n2)cc1OC]

### 5-(3,4-Dimethoxyphenyl)-2-(thiophen-2-yl)-7-(trifluoromethyl)pyrazolo[1,5-a]pyrimidine

One embodiment of the present invention is a compound of formula IV for use in treatment and/or prevention of Hepatitis B virus (HBV) infection.

One embodiment of the present invention is a compound of formula V, VI or VI for use in treatment and/or prevention of Hepatitis B virus (HBV) infection.

The S-adenosyl-L-methionine (SAM) competitive inhibitor, GSK343 (formula VIII), is currently in preclinical development for osteosarcoma. It has been shown that GSK343 inhibits FUBP1 expression in osteosarcoma cells (Xiong et al 2016 Int J Onc vol 49 p 623).

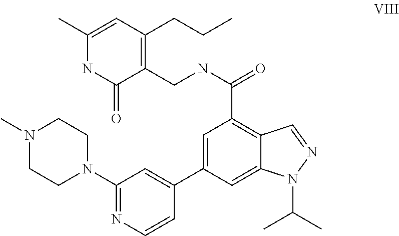 [SMILES: CCCc1cc(C)[nH]c(=O)c1CNC(=O)c1cc(-c2ccnc(N3CCN(C)CC3)c2)cc2c1cnn2C(C)C]

One embodiment of the present invention is a compound of formula VII for use in treatment and/or prevention of Hepatitis B virus (HBV) infection.

The FDA-approved cancer drugs camptothecin (CPT, formula IX) and its derivative SN-38 (7-ethyl-10-hydroxycamptothecin, formula X), which are Topoisomerase I (TOP1) inhibitors, were recently shown to inhibit FUBP1 activity as well by preventing the FUBP1/FUSE interaction (Hosseini et al 2017 Biochemical Pharmacology Vol 146 p. 53-62).

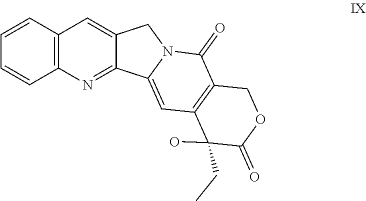 [SMILES: CC[C@@]1(O)C(=O)OCc2c1cc1n(c2=O)Cc2cc3ccccc3nc2-1]

### Camptothecin ((+)-4(S)-Ethyl-4-hydroxy-3,4,12,14-tetrahydro-1H-pyrano[3′,4′:6,7]indolizino[1,2-b]quinoline-3,14-dione)

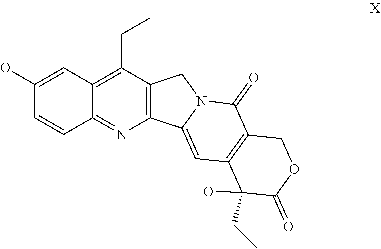 [SMILES: CCc1c2c(nc3ccc(O)cc13)-c1cc3c(c(=O)n1C2)COC(=O)[C@]3(O)CC]

### SN-38 (4(S),11-Diethyl-4,9-dihydroxy-3,4,12,14-tetrahydro-1H-pyrano[3′,4′:6,7]indolizino[1,2-b]quinoline-3,14-dione 7-Ethyl-10-hydroxycamptothecin)

One embodiment of the present invention is a compound of formula IX or X for use in treatment and/or prevention of Hepatitis B virus (HBV) infection.

Tringali et al 2012 Journal of Pharmacy and Pharmacology Vol 64, p. 360-365 describes the pharmacokinetic profile SN-38 conjugated to hyaluronic acid (HA-SN-38, formula XI) and shows an increased distribution to the liver.

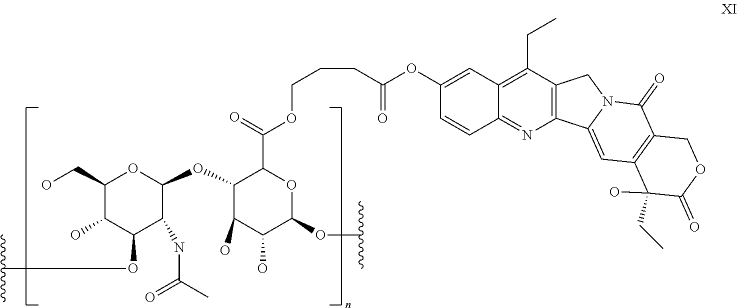

One embodiment of the present invention is a compound of formula XI for use in treatment and/or prevention of Hepatitis B virus (HBV) infection.

Various lipid conjugates of SN-38 also exist in the literature. WO2006/082053 for example describes the molecule of formula XII

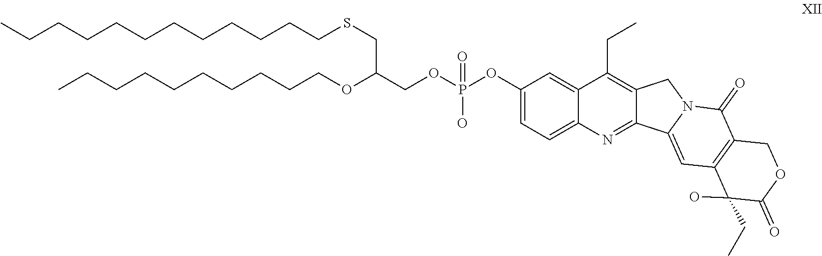 [SMILES: CCCCCCCCCCCCSCC(COP(=O)(O)Oc1ccc2nc3c(c(CC)c2c1)Cn1c-3cc2c(c1=O)COC(=O)[C@]2(O)CC)OCCCCCCCCCC]

CN105777770 describes a palmitate conjugated SN-38 shown in formula XIII below.

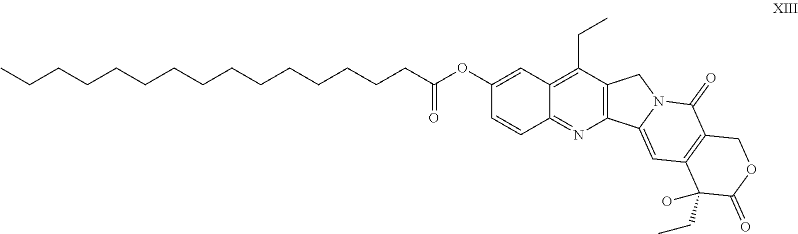 [SMILES: CCCCCCCCCCCCCCCC(=O)Oc1ccc2nc3c(c(CC)c2c1)Cn1c-3cc2c(c1=O)COC(=O)[C@]2(O)CC]

One embodiment of the present invention is a compound of formula XII or XIII for use in treatment and/or prevention of Hepatitis B virus (HBV) infection.

In a further aspect of the invention the FUBP1 inhibitors for use in treatment and/or prevention of Hepatitis B virus (HBV) infection can be targeted directly to the liver by covalently attaching them to a conjugate moiety capable of binding to the asialoglycoprotein receptor (ASGPr), such as divalent or trivalent GalNAc cluster.

### Nucleic Acid Molecules of the Invention

Nucleic acid molecules are potentially excellent FUBP1 inhibitors since they can target the FUBP1 transcript and promote its degradation either via the RNA interference pathway or via RNaseH cleavage. Alternatively, nucleic acid molecules such as aptamers can also act as inhibitors of the DNA binding site of FUBP1 in line with the small molecules described above.

One aspect of the present invention is a nucleic acid molecule for use in treatment and/or prevention of Hepatitis B virus (HBV) infection. Such nucleic acid molecules can be selected from the group consisting of single stranded antisense, oligonucleotide; siRNA molecule; or shRNA molecule.

The present section describes novel nucleic acid molecules suitable for use in treatment and/or prevention of Hepatitis B virus (HBV) infection.

The nucleic acid molecules of the present invention are capable of inhibiting the expression of FUBP1 in vitro and in vivo. The inhibition is achieved by hybridizing an oligonucleotide to a target nucleic acid encoding FUBP1.

The target nucleic acid may be a mammalian FUBP1 sequence, such as a sequence selected from the group consisting of SEQ ID NO: 1 to 20. It is advantageous if the mammalian FUBP1 sequence is selected from the group consisting of SEQ ID NO: 1, 2, 3, 4, 5, 6, 7 and 8.

In some embodiments, the nucleic acid molecule of the invention is capable of modulating the expression of FUBP1 by inhibiting or down-regulating it. Preferably, such modulation produces an inhibition of expression of at least 40% compared to the normal expression level of the target, more preferably at least 50%, 60%, 70%, 80%, 90%, 95% or 98% inhibition compared to the normal expression level of the target. In some embodiments, the nucleic acid molecule of the invention is capable of inhibiting expression levels of FUBP1 mRNA by at least 65% -98%, such as 70% to 95%, in vitro using HepG2-NTCP cells or HBV infected primary human hepatocytes, this range of target reduction is advantageous in terms of selecting nucleic acid molecules with good correlation to the cccDNA reduction. In some embodiments compounds of the invention may be capable of inhibiting expression levels of FUBP1 protein by at least 50% in vitro using HepG2-NTCP cells or HBV infected primary human hepatocytes. The materials and Method section and the Examples herein provide assays which may be used to measure target RNA inhibition in HepG2-NTCP cells or HBV infected primary human hepatocytes as well as cccDNA. The target modulation is triggered by the hybridization between a contiguous nucleotide sequence of the oligonucleotide, such as the guide strand of a siRNA or gapmer region of an antisense oligonucleotide, and the target nucleic acids. In some embodiments, the oligonucleotide of the invention comprises mismatches between the oligonucleotide or the contiguous nucleotide sequence and one or both of the target nucleic acids. Despite mismatches hybridization to the target nucleic acid may still be sufficient to show a desired modulation of FUBP1 expression. Reduced binding affinity resulting from mismatches may advantageously be compensated by increased length of the oligonucleotide and/or an increased number of modified nucleosides capable of increasing the binding affinity to the target within the oligonucleotide sequence. Advantageously, the oligonucleotides of the present invention contain modified nucleosides capable of increasing the binding affinity, such as 2′ sugar modified nucleosides, including LNA.

An aspect of the present invention relates to nucleic acid molecule of 12 to 60 nucleotides in length, which comprises a contiguous nucleotide sequence of 12 to 30 nucleotides in length which is capable of inhibiting the expression of FUBP1.

In some embodiments, the nucleic acid molecule comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

In one embodiment the nucleic acid molecule of the invention, or contiguous nucleotide sequence thereof is fully complementary (100% complementary) to a region of the target nucleic acids, or in some embodiments may comprise one or two mismatches between the oligonucleotide and the target nucleic acids.

In some embodiments, the nucleic acid molecule comprises a contiguous nucleotide sequence of 12 to 30 nucleotides in length with at least 95% complementary, such as fully (or 100%) complementary, to a target nucleic acid region present in SEQ ID NO: 1, SEQ ID NO: 2, SEQ ID NO: 3 and/or SEQ ID NO:4.

In some embodiments, the nucleic acid molecule or the contiguous nucleotide sequence of the invention is at least 93% complementarity, such as fully (or 100%) complementary, to the target nucleic acid of SEQ ID NO: 1, SEQ ID NO: 2, SEQ ID NO: 3, SEQ ID NO: 4, SEQ ID NO: 5, SEQ ID NO: 6, SEQ ID NO; 7 and /or SEQ ID NO; 8.

In some embodiments the nucleic acid molecule or the contiguous nucleotide sequence of the invention is at least 95% complementarity, such as fully (or 100%) complementary, to the target nucleic acid of SEQ ID NO: 1 and SEQ ID NO: 5.

In some embodiments the nucleic acid molecule or the contiguous nucleotide sequence of the invention is at least 95% complementarity, such as fully (or 100%) complementary, to the target nucleic acid of SEQ ID NO: 1, SEQ ID NO: 5 and SEQ ID NO: 9.

In some embodiments the nucleic acid molecule or the contiguous nucleotide sequence is 100% complementary to position 14200-14218 on SEQ ID NO: 1.

In some embodiments the nucleic acid molecule or the contiguous nucleotide sequence is 100% complementary to position 14413-14431 on SEQ ID NO: 1.

In some embodiments the nucleic acid molecule or the contiguous nucleotide sequence is 100% complementary to position 14966-14984 on SEQ ID NO: 1.

In some embodiments the nucleic acid molecule or the contiguous nucleotide sequence is 100% complementary to position 30344-30362 on SEQ ID NO: 1

In some embodiments, the nucleic acid molecule of the invention comprises or consists of 12 to 60 nucleotides in length, such as from 13 to 50, such as 14 to 35, such as from 15 to 30 such as from 16 to 22 nucleotides in length.

In some embodiments, the contiguous nucleotide sequence of the acid molecule which is complementary to the target nucleic acids comprises or consists of 12 to 30, such as from 14 to 25, such as from 16 to 23, such as from 18 to 22, contiguous nucleotides in length.

In some embodiments, the contiguous nucleotide sequence of the siRNA or shRNA which is complementary to the target nucleic acids comprises or consists of 18 to 28, such as from 19 to 26, such as from 20 to 24, such as from 21 to 23, contiguous nucleotides in length.

In some embodiments, the contiguous nucleotide sequence of the antisense oligonucleotide which is complementary to the target nucleic acids comprises or consists of 12 to 22, such as from 14 to 20, such as from 16 to 20, such as from 15 to 18, such as from 16 to 18, such as from 16 to 17 contiguous nucleotides in length.

It is understood that the contiguous nucleobase sequences (motif sequence) can be modified to for example increase nuclease resistance and/or binding affinity to the target nucleic acid.

The pattern in which the modified nucleosides (such as high affinity modified nucleosides) are incorporated into the oligonucleotide sequence is generally termed oligonucleotide design. The oligonucleotides of the invention are designed with modified nucleosides and RNA nucleosides (in particular for siRNA and shRNA molecules) or DNA nucleosides (in particular for single stranded antisense oligonucleotides). Advantageously, high affinity modified nucleosides are used.

In an embodiment, the oligonucleotide comprises at least 1 modified nucleoside, such as at least 2, at least 3, at least 4, at least 5, at least 6, at least 7, at least 8 modified nucleosides. In an embodiment the oligonucleotide comprises from 1 to 8 modified nucleosides, such as from 2 to 7 modified nucleosides, such as from 3 to 6 modified nucleosides, such as from 4 to 6 modified nucleosides, such as 4 or 5 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide of the invention comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA). Often used LNA nucleosides are oxy-LNA or cET.

In a further embodiment the oligonucleotide comprises at least one modified internucleoside linkage. Suitable internucleoside modifications are described in the “Definitions” section under “Modified internucleoside linkage”. It is advantageous if at least 2 to 3 internucleoside linkages at the 5′ or 3′ end of the oligonucleotide are phosphorothioate internucleoside linkages. For single stranded antisense oligonucleotides it is advantageous if at least 75%, such as, such as all, the internucleoside linkages within the contiguous nucleotide sequence are phosphorothioate internucleoside linkages.

In a further aspect of the invention the nucleic acid molecules, such as the antisense oligonucleotide, siRNA or shRNA, of the invention can be targeted directly to the liver by covalently attaching them to a conjugate moiety capable of binding to the asialoglycoprotein receptor (ASGPr), such as divalent or trivalent GalNAc cluster.

### Conjugates

Since HBV infection primarily affects the hepatocytes in the liver it is advantageous to conjugate the FUBP1 inhibitor to a conjugate moiety that will increase the delivery of the inhibitor to the liver compared to the unconjugated inhibitor. In one embodiment liver targeting moieties are selected from moieties comprising cholesterol or other lipids or conjugate moieties capable of binding to the asialoglycoprotein receptor (ASGPR).

In some embodiments, the invention provides a conjugate comprising a nucleic acid molecule of the invention covalently attached to a conjugate moiety.

The asialoglycoprotein receptor (ASGPR) conjugate moiety comprises one or more carbohydrate moieties capable of binding to the asialoglycoprotein receptor (ASPGR targeting moieties) with affinity equal to or greater than that of galactose. The affinities of numerous galactose derivatives for the asialoglycoprotein receptor have been studied (see for example: Jobst, S. T. and Drickamer, K. J B. C. 1996, 271, 6686) or are readily determined using methods typical in the art.

In one embodiment the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine. Advantageously the asialoglycoprotein receptor targeting moiety is N-acetylgalactosamine (GalNAc).

To generate the ASGPR conjugate moiety the ASPGR targeting moieties (preferably GalNAc) can be attached to a conjugate scaffold. Generally, the ASPGR targeting moieties can be at the same end of the scaffold. In one embodiment, the conjugate moiety consists of two to four terminal GalNAc moieties linked to a spacer which links each GalNAc moiety to a brancher molecule that can be conjugated to the antisense oligonucleotide.

In a further embodiment, the conjugate moiety is mono-valent, di-valent, tri-valent or tetra-valent with respect to asialoglycoprotein receptor targeting moieties. Advantageously the asialoglycoprotein receptor targeting moiety comprises N-acetylgalactosamine (GalNAc) moieties.

GalNAc conjugate moieties can include, for example, those described in WO 2014/179620 and WO 2016/055601 and PCT/EP2017/059080 (hereby incorporated by reference), as well as small peptides with GalNAc moieties attached such as Tyr-Glu-Glu-(aminohexyl GalNAc)3 (YEE(ahGalNAc)3; a glycotripeptide that binds to asialoglycoprotein receptor on hepatocytes, see, e.g., Duff, et al., Methods Enzymol, 2000, 313, 297); lysine-based galactose clusters (e.g., L3G4; Biessen, et al., Cardovasc. Med., 1999, 214); and cholane-based galactose clusters (e.g., carbohydrate recognition motif for asialoglycoprotein receptor).

The ASGPR conjugate moiety, in particular a trivalent GalNAc conjugate moiety, may be attached to the 3′- or 5′-end of the oligonucleotide using methods known in the art. In one embodiment the ASGPR conjugate moiety is linked to the 5′-end of the oligonucleotide.

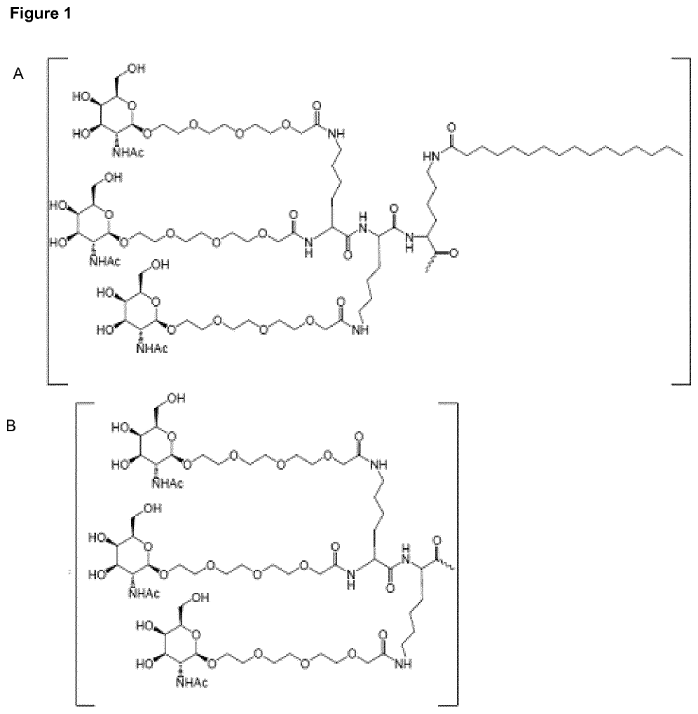

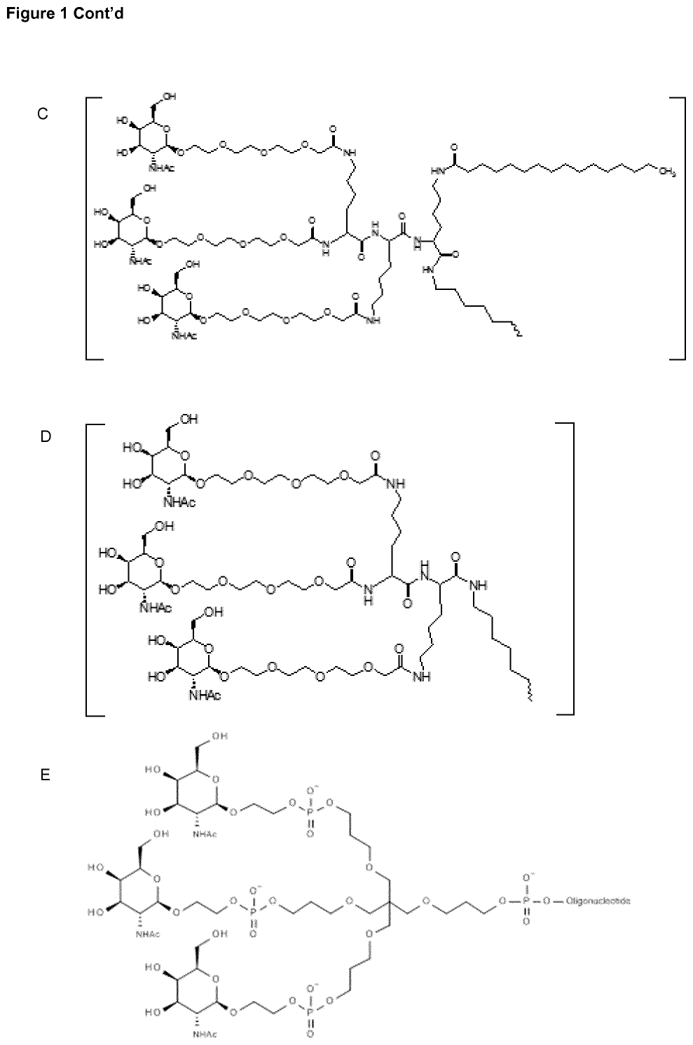

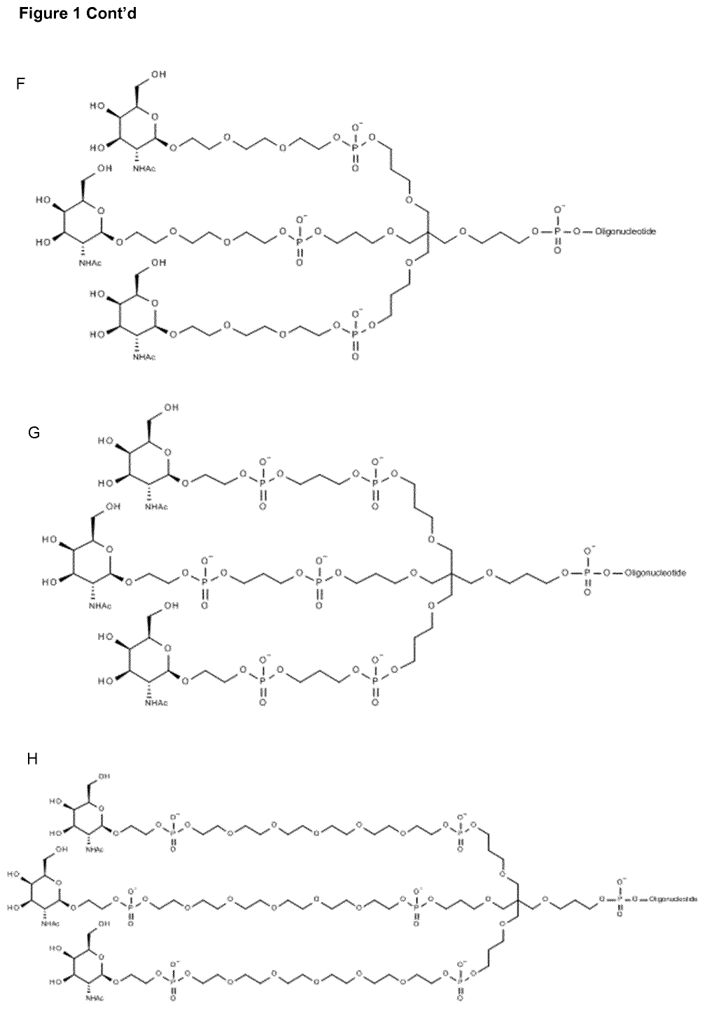

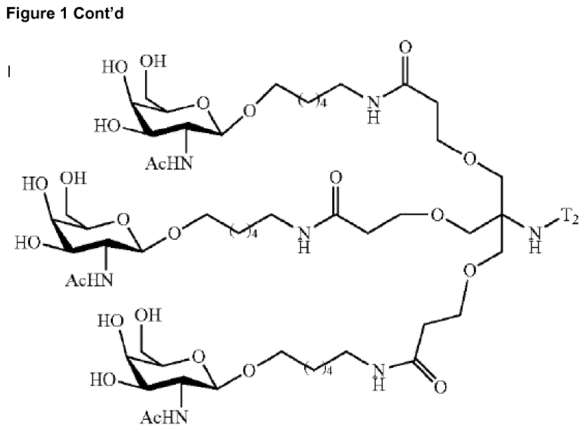

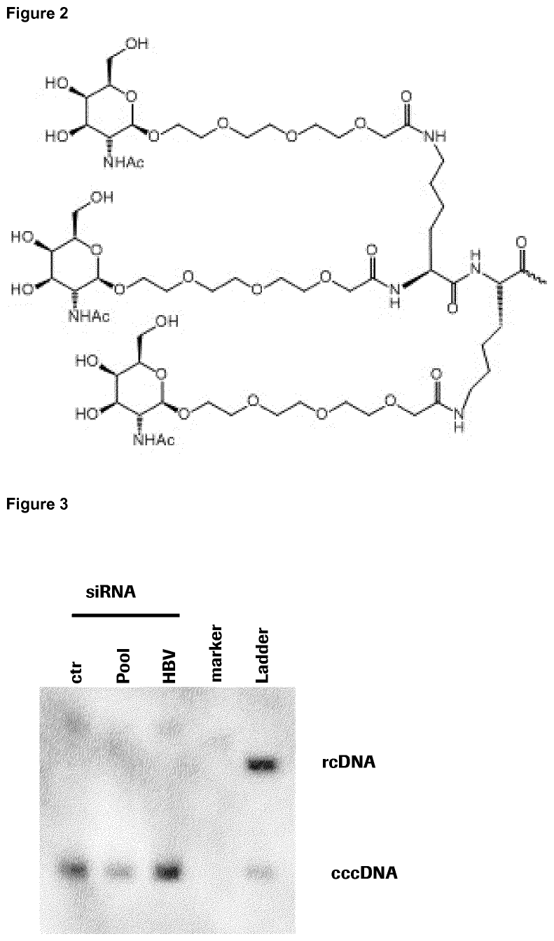

In one embodiment the conjugate moiety is a tri-valent N-acetylgalactosamine (GalNAc), such as those shown in FIG. 1, in particular as shown in FIG. 1D.

### Method of Manufacture

In a further aspect, the invention provides methods for manufacturing the nucleic acid molecule of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the oligonucleotide with a conjugating moiety (ligand) to covalently attach the conjugate moiety to the oligonucleotide. In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant. Where the nucleic acid molecule is a siRNA the oligonucleotides are paired and allowed to form double stranded structures.

### Pharmaceutical Salt

The compounds according to the present invention may exist in the form of their pharmaceutically acceptable salts. The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

In a further aspect the invention provides a pharmaceutically acceptable salt of the antisense oligonucleotide or a conjugate thereof. In a preferred embodiment, the pharmaceutically acceptable salt is a sodium or a potassium salt.

### Pharmaceutical Compositions

In a further aspect, the invention provides pharmaceutical compositions comprising a nucleic acid molecule and/or conjugate compounds of the invention or salts thereof and a pharmaceutically acceptable diluent, carrier, salt and/or adjuvant. A pharmaceutically acceptable diluent includes phosphate-buffered saline (PBS). In some embodiments the pharmaceutically acceptable diluent is sterile phosphate buffered saline. In some embodiments the oligonucleotide is used in the pharmaceutically acceptable diluent at a concentration of 50 -300 μM solution.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Nucleic acid molecules or conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the nucleic acid molecule or conjugate of the invention is a prodrug. In particular, with respect to oligonucleotide conjugates the conjugate moiety is cleaved off the oligonucleotide once the prodrug is delivered to the site of action, e.g. the target cell.

### Applications

The nucleic acid molecules of the invention may be utilized as research reagents for, for example, diagnostics, therapeutics and prophylaxis.

In research, such nucleic acid molecules may be used to specifically modulate the synthesis of FUBP1 protein in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby prevent protein formation or by degrading or inhibiting a modulator of the gene or mRNA producing the protein.

If employing the nucleic acid molecules of the invention in research or diagnostics the target nucleic acid may be a cDNA or a synthetic nucleic acid derived from DNA or RNA.

Also encompassed by the present invention is an in vivo or in vitro method for modulating FUBP1 expression in a target cell which is expressing FUBP1, said method comprising administering a nucleic acid molecule, conjugate compound or pharmaceutical composition of the invention in an effective amount to said cell.

In some embodiments, the target cell, is a mammalian cell in particular a human cell. The target cell may be an in vitro cell culture or an in vivo cell forming part of a tissue in a mammal. In preferred embodiments the target cell is present in in the liver. The target cell may be a hepatocyte.

One aspect of the present invention is related the nucleic acid molecules, conjugate compounds or pharmaceutical compositions of the invention for use as a medicament.

In an aspect of the invention the nucleic acid molecules, conjugate compound or pharmaceutical composition of the invention is capable of reducing the cccDNA level in the infected cells and therefore inhibiting HBV infection. In particular, the antisense oligonucleotide is capable of affecting one or more of the following parameters i) reducing cccDNA and/or ii) reducing pgRNA and/or iii) reducing HBV DNA and/or iv) reducing HBV viral antigens in an infected cell.

For example, nucleic acid molecule that inhibits HBV infection may reduce i) the cccDNA levels in an infected cell by at least 40% such as 50%, 60%, 70%, 80%, or 90% reduction compared to controls; or ii) the level of pgRNA by at least 40% such as 50%, 60%, 70%, 80%, or 90% reduction compared to controls. The controls may be untreated cells or animals, or cells or animals treated with an appropriate control.

Inhibition of HBV infection may be measured in vitro using HBV infected primary human hepatocytes or in vivo for nucleic acid molecules complementary to mouse FUBP1 using HBV minicircle mouse (available at Covance Shanghai, see also Guo et al 2016 Sci Rep 6: 2552 and Yan et al 2017 J Hepatology 66(6):1149-1157) or humanized hepatocytes PXB mouse model (available at PhoenixBio, see also Kakuni et al 2014 Int. J. Mol. Sci. 15:58-74). Inhibition of secretion of HBsAg and/or HBeAg may be measured by ELISA, e.g. by using the CLIA ELISA Kit (Autobio Diagnostic) according to the manufacturers' instructions. Reduction of intracellular cccDNA or HBV mRNA and pgRNA may be measured by qPCR, e.g. as described in the Materials and Methods section. Further methods for evaluating whether a test compound inhibits HBV infection are measuring secretion of HBV DNA by qPCR e.g. as described in WO 2015/173208 or using Northern Blot; in-situ hybridization, or immuno-fluorescence.

Due to the reduction of FUBP1 levels the nucleic acid molecules, conjugate compounds or pharmaceutical compositions of the present invention can be used to inhibit development of or in the treatment of HBV infection. In particular, the destabilization and reduction of the cccDNA, the nucleic acid molecules, conjugate compounds or pharmaceutical compositions of the present invention more efficiently inhibits development of or treats a chronic HBV infection as compared to a compound that only reduces secretion of HBsAg.

Accordingly, one aspect of the present invention is related to use of the nucleic acid molecule, conjugate compounds or pharmaceutical compositions of the invention to reduce cccDNA and/or pgRNA in an HBV infected individual.

A further aspect of the invention relates to the use of the nucleic acid molecules, conjugate compounds or pharmaceutical compositions of the invention to inhibit development of or treat a chronic HBV infection.

A further aspect of the invention relates to the use of the nucleic acid molecules, conjugate compounds or pharmaceutical compositions of the invention to reduce the infectiousness of a HBV infected person. In a particular aspect of the invention, the nucleic acid molecules, conjugate compounds or pharmaceutical compositions of the invention inhibits development of a chronic HBV infection.

The subject to be treated with the nucleic acid molecules, conjugate compounds or pharmaceutical compositions of the invention (or which prophylactically receives antisense oligonucleotides, conjugate compounds or pharmaceutical compositions of the present invention) is preferably a human, more preferably a human patient who is HBsAg positive and/or HBeAg positive, even more preferably a human patient that is HBsAg positive and HBeAg positive.

Accordingly, the present invention relates to a method of treating and/or preventing a HBV infection, wherein the method comprises administering an effective amount of the nucleic acid molecules, conjugate compounds or pharmaceutical compositions of the invention.

The invention also provides for the use of a nucleic acid molecule, a conjugate compound or a pharmaceutical composition of the invention for the manufacture of a medicament, in particular a medicament for use in the treatment or prevention of HBV infection or chronic HBV infection or reduction of the infectiousness of a HBV infected person. In preferred embodiments the medicament is manufactured in a dosage form for subcutaneous administration.

The invention also provides for the use of a nucleic acid molecule, a conjugate compound, the pharmaceutical composition of the invention for the manufacture of a medicament wherein the medicament is in a dosage form for intravenous administration.

The nucleic acid molecule, conjugate or the pharmaceutical composition of the invention may be used in a combination therapy. For example, nucleic acid molecule, conjugate or the pharmaceutical composition of the invention may be combined with other anti-HBV agents such as interferon alpha-2b, interferon alpha-2a, and interferon alphacon-1 (pegylated and unpegylated), ribavirin, lamivudine (3TC), entecavir, tenofovir, telbivudine (LdT), adefovir, or other emerging anti-HBV agents such as a HBV RNA replication inhibitor, a HBsAg secretion inhibitor, a HBV capsid inhibitor, an antisense oligomer (e.g. as described in WO2012/145697, WO 2014/179629 and WO2017/216390), a siRNA (e.g. described in WO 2005/014806, WO 2012/024170, WO 2012/2055362, WO 2013/003520, WO 2013/159109, WO 2017/027350 and WO2017/015175), a HBV therapeutic vaccine, a HBV prophylactic vaccine, a HBV antibody therapy (monoclonal or polyclonal), or TLR 2, 3, 7, 8 or 9 agonists for the treatment and/or prophylaxis of HBV.

### Administration

The nucleic acid molecule, conjugate compounds or pharmaceutical composition of the invention is formulated, dosed, and administered in a fashion consistent with good medical practice. Factors for consideration in this context include the particular mammal being treated, the clinical condition of the individual patient, the site of delivery of the agent, the method of administration, the scheduling of administration, the age and sex of the patients and other factors known to medical practitioners. Herein, an “effective amount” (also known as “(therapeutically) effective dose”) means the amount of a compound that will elicit the biological or medical response of a subject that is being sought by a medical doctor or other clinician. The “effective amount” of an nucleic acid molecule, conjugate compound or pharmaceutical composition of the invention, will be governed by such considerations, and is the minimum amount necessary to inhibit HBsAg and/or HBeAg. For example, such amount may be below the amount that is toxic to the cells of the recipient, or to the mammal as a whole.

In some embodiments, the nucleic acid molecule, conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

The nucleic acid molecules, conjugates or pharmaceutical compositions of the present invention may be administered topical (such as, to the skin, inhalation, ophthalmic or otic) or enteral (such as, orally or through the gastrointestinal tract) or parenteral (such as, intravenous, subcutaneous, intra-muscular, intracerebral, intracerebroventricular or intrathecal).

In a preferred embodiment the nucleic acid molecule, conjugate compounds or pharmaceutical compositions of the present invention are administered by a parenteral route including intravenous, intraarterial, subcutaneous, intraperitoneal or intramuscular injection or infusion. In one embodiment the active oligonucleotide or oligonucleotide conjugate is administered intravenously. With GalNAc conjugated compounds it may be advantageous to administer subcutaneously in order to delay saturation of the ASGP receptor.

### Combination Therapies

In some embodiments the oligonucleotide, oligonucleotide conjugate or pharmaceutical composition of the invention is for use in a combination treatment with another therapeutic agent. The therapeutic agent can for example be the standard of care for the diseases or disorders described above.

By way of example, the oligomer or the oligomer conjugate of the present invention may be used in combination with other actives, such as oligonucleotide-based antivirals—such as sequence specific oligonucleotide-based antivirals—acting either through antisense (including LNA antisense oligonucleotides as described in WO 2011/047312 or WO2015/173208 or MOE antisense oligonucleotides such as GSK3389404 or those described in WO 2012/145697 or WO 2014/79629), siRNAs (such as ARC-520, ARC-521, ARB-1467 or those described in WO2 013/003520, WO2016/077321, US 2017/0016000 or WO 2017/027350), aptamers, morpholinos or any other antiviral, nucleotide sequence-dependent mode of action.

By way of further example, the oligomer or the oligomer conjugate of the present invention may be used in combination with other actives, such as immune stimulatory antiviral compounds, such as interferon (e.g. pegylated interferon alpha), TLR7 agonists (e.g. GS-9620), TLR8 agonists (e.g. GS-9688) or therapeutic vaccines.

By way of further example, the oligomer or the oligomer conjugate of the present invention may be used in combination with other actives, such as small molecules, with antiviral activity. These other actives could be, for example, nucleoside/nucleotide inhibitors (eg entecavir or tenofovir disoproxil fumarate), encapsidation inhibitors, entry inhibitors (eg Myrcludex B).

In certain embodiments, the additional therapeutic agent may be an HBV agent, an Hepatitis C virus (HCV) agent, a chemotherapeutic agent, an antibiotic, an analgesic, a nonsteroidal anti-inflammatory (NSAID) agent, an antifungal agent, an antiparasitic agent, an anti-nausea agent, an anti-diarrheal agent, or an immunosuppressant agent.

In particular related embodiments, the additional HBV agent may be interferon alpha-2b, interferon alpha-2a, and interferon alphacon-1 (pegylated and unpegylated), ribavirin; an HBV RNA replication inhibitor; a second antisense oligomer; an HBV therapeutic vaccine; an HBV prophylactic vaccine; lamivudine (3TC); entecavir (ETV); tenofovir diisoproxil fumarate (TDF); telbivudine (LdT); adefovir; or an HBV antibody therapy (monoclonal or polyclonal).

In other particular related embodiments, the additional HCV agent may be interferon alpha-2b, interferon alpha-2a, and interferon alphacon-1 (pegylated and unpegylated); ribavirin; pegasys; an HCV RNA replication inhibitor (e.g., ViroPharma's VP50406 series); an HCV antisense agent; an HCV therapeutic vaccine; an HCV protease inhibitor; an HCV helicase inhibitor; or an HCV monoclonal or polyclonal antibody therapy.

### Embodiments of the Invention

The following embodiments of the present invention may be used in combination with any other embodiments described herein.
1. A FUBP1 inhibitor for use in the in the treatment and/or prevention of Hepatitis B virus (HBV) infection.
2. The FUBP1 inhibitor for the use of embodiment 1, wherein the FUBP1 inhibitor is administered in an effective amount.
3. The FUBP1 inhibitor for the use of embodiment 1 or 2, wherein the HBV infection is a chronic infection.
4. The FUBP1 inhibitor for the use of embodiments 1 to 3, wherein the FUBP1 inhibitor is capable of reducing cccDNA and/or pgRNA in an infected cell.
5. The FUBP1 inhibitor for the use of any one of embodiments 1 to 4, wherein the FUBP1 inhibitor prevents or reduces the binding of FUBP1 to DNA, such as cccDNA.
6. The FUBP1 inhibitor for the use of any one of embodiments 1 to 5, wherein the inhibitor prevents or reduces the FUBP1/FUSE interaction.
7. The FUBP1 inhibitor for the use of any one of embodiments 1 to 6, wherein the inhibitor interacts with the DNA binding domain of FUBP1 protein.
8. The FUBP1 inhibitor for the use of any one of embodiments 1 to 6, wherein the inhibitor interacts with a FUSE element on cccDNA.
9. FUBP1 inhibitor for the use of embodiment 6 to 8 , wherein said inhibitor is a small molecule that specifically prevents or reduces binding of FUBP1 protein to cccDNA.
10. The FUBP1 inhibitor for the use of any one of claims 1 to 9, wherein the inhibitor is selected from the compounds of Formula VII, IX or X

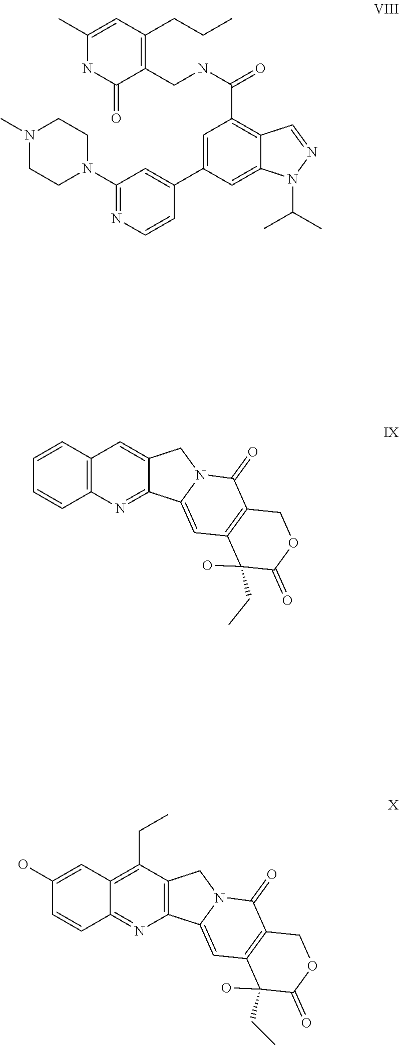 [SMILES: CCCc1cc(C)[nH]c(=O)c1CNC(=O)c1cc(-c2ccnc(N3CCN(C)CC3)c2)cc2c1cnn2C(C)C ; CC[C@@]1(O)C(=O)OCc2c1cc1n(c2=O)Cc2cc3ccccc3nc2-1 ; CCc1c2c(nc3ccc(O)cc13)-c1cc3c(c(=O)n1C2)COC(=O)[C@]3(O)CC]

11. The FUBP1 inhibitor for the use of any one of embodiments 1 to 5, wherein said inhibitor is a genome editing machinery, comprising:
   a. a site-specific DNA nuclease or a polynucleotide encoding a site-specific DNA nuclease; and
   b. a guide RNA or a polynucleotide encoding a guide RNA.
12. The FUBP1 inhibitor for the use of any one of embodiments 1 to 5, wherein said inhibitor is a nucleic acid molecule comprising or consisting of a contiguous nucleotide sequence of 12 to 30 nucleotides in length which is at least 90% complementary to a mammalian FUBP1 target nucleic acid and capable of reducing the level of the target nucleic acid.
13. The FUBP1 inhibitor for the use of embodiment 12, wherein the target nucleic acid is RNA.
14. The FUBP1 inhibitor for the use of embodiment 13, wherein the RNA is pre-mRNA.
15. The FUBP1 inhibitor for the use of any one of embodiments 11 to14, wherein the nucleic acid molecule is selected from a antisense oligonucleotide, siRNA or shRNA.
16. The FUBP1 inhibitor for the use of embodiments 15, wherein the nucleic acid molecule is a single stranded antisense oligonucleotide or a double stranded siRNA.
17. The nucleic acid molecule for the use of any one of embodiments 12 to 16, wherein the mammalian FUBP1 target nucleic acid is selected from SEQ ID NO: 1 to 20.
18. The nucleic acid molecule for the use of embodiment 12 to 16, wherein the mammalian FUBP1 target nucleic acid is selected from SEQ ID NO: 1 to 8.
19. The nucleic acid molecule for the use of embodiment 12 to 16, wherein the mammalian FUBP1 target nucleic acid is selected from SEQ ID NO: 1 to 4.
20. The nucleic acid molecule for the use of any one of embodiments 12 to 16, wherein the contiguous nucleotide sequence is at least 98% complementarity to the target nucleic acid of SEQ ID NO: 1 and SEQ ID NO: 5 and SEQ ID NO: 9.
21. The nucleic acid molecule for the use of any one of embodiments 12 to 16, wherein the contiguous nucleotide sequence is at least 98% complementarity to the target nucleic acid of SEQ ID NO: 1 and SEQ ID NO: 5.
22. The FUBP1 inhibitor for the use of any one of embodiments 1 to 21, wherein the cccDNA in an HBV infected cell is reduced by at least 50%, such as 60%, such as 70%, such as 80%, such 90%, such as 95%, such as 100%, when compared to a control.
23. The nucleic acid molecule for the use of any one of embodiments 12 to 22, wherein the FUBP1 mRNA is reduced by at least 50%, such as 60%, such as 70%, such as 80%, such as 90%, such as 95%, such as 100%, when compared to a control.
24. A nucleic acid molecule of 12 to 60 nucleotides in length which comprises or consists of a contiguous nucleotide sequence of 12 to 30 nucleotides in length wherein the contiguous nucleotide sequence is at least 90% complementary, such as 95%, such as 98%, such as fully complementarity, to a mammalian FUBP1 target nucleic acid.
25. The nucleic acid molecule of embodiment 24, wherein the nucleic acid molecule is chemically produced
26. The nucleic acid molecule of embodiment 24 or 25, wherein the mammalian FUBP1 target nucleic acid is selected from SEQ ID NO: 1 to 20.
27. The nucleic acid molecule of embodiment 24 or 25, wherein the mammalian FUBP1 target nucleic acid is selected from SEQ ID NO: 1 to 8.
28. The nucleic acid molecule of embodiment 24 or 25, wherein the mammalian FUBP1 target nucleic acid is selected from SEQ ID NO: 1 to 4.
29. The nucleic acid molecule embodiment 24 or 25, wherein the contiguous nucleotide sequence is at least 98% complementarity to the target nucleic acid of SEQ ID NO: 1 and SEQ ID NO: 5 and SEQ ID NO: 9.
30. The nucleic acid molecule of embodiment 24 or 25, wherein the contiguous nucleotide sequence is at least 98% complementarity to the target nucleic acid of SEQ ID NO: 1 and SEQ ID NO: 5.
31. The nucleic acid molecule of any one of embodiments 24 to 30, wherein the nucleic acid molecule is 12 to 30 nucleotides in length.
32. The nucleic acid molecule of any one of embodiments 24 to 31, wherein the nucleic acid molecule is a double stranded siRNA or a single stranded antisense oligonucleotide.
33. The nucleic acid molecule of any one of embodiments 24 to 31, wherein contiguous nucleotide sequence is complementary to a target sequence selected from the group consisting of position 14200-14218, 14413-14431, 14966-14984 and 30344-30362 on SEQ ID NO: 1.
34. The nucleic acid molecule of embodiment 24 to 33, which is capable of hybridizing to a target nucleic acid of SEQ ID NO: 1 and SEQ ID NO: 5 with a ΔG° below −15 kcal.
35. The nucleic acid molecule of any one of embodiments 24 to 34, wherein the contiguous nucleotide sequence comprises or consists of at least 14 contiguous nucleotides, particularly 15, 16, 17, 18, 19, 20, 21 or 22 contiguous nucleotides.
36. The nucleic acid molecule of any one of embodiments 24 to 34, wherein the contiguous nucleotide sequence comprises or consists of from 14 to 22 nucleotides.
37. The nucleic acid molecule of embodiment 36, wherein the contiguous nucleotide sequence comprises or consists of from 16 to 18 nucleotides.
38. The nucleic acid molecule of embodiment 24 to 37, wherein the nucleic acid molecule comprises or consists of 14 to 25 nucleotides in length.
39. The nucleic acid molecule of embodiment 38, wherein the nucleic acid molecule comprises or consists of 16 to 22 nucleotides in length.
40. The nucleic acid molecule of any one of embodiments 24 to 39, wherein the contiguous nucleotide sequence has zero to three mismatches compared to the target nucleic acids it is complementary to.
41. The nucleic acid molecule of embodiment 40, wherein the contiguous nucleotide sequence has one mismatch compared to the target nucleic acids.
42. The nucleic acid molecule of embodiment 40, wherein the contiguous nucleotide sequence has two mismatches compared to the target nucleic acids.
43. The nucleic acid molecule of embodiment 40, wherein the contiguous nucleotide sequence is fully complementary to both target nucleic acid sequences.
44. The nucleic acid molecule of embodiment 24 to 43, comprising one or more modified nucleosides.
45. The nucleic acid molecule of embodiment 44, wherein the one or more modified nucleoside is a high-affinity modified nucleosides.
46. The nucleic acid molecule of embodiment 44 or 45, wherein the one or more modified nucleoside is a 2′ sugar modified nucleoside.
47. The nucleic acid molecule of embodiment 46, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, 2′-fluoro-ANA and LNA nucleosides.
48. The nucleic acid molecule of embodiment 44-47, wherein the one or more modified nucleoside is a LNA nucleoside.
49. The nucleic acid molecule of embodiment 48, wherein the modified LNA nucleoside is selected from oxy-LNA, amino-LNA, thio-LNA, cET, and ENA.
50. The nucleic acid molecule of embodiment 48 or 49, wherein the modified LNA nucleoside is oxy-LNA with the following 2′-4′ bridge —O—CH₂—.
51. The nucleic acid molecule of embodiment 50, wherein the oxy-LNA is beta-D-oxy-LNA.
52. The nucleic acid molecule of embodiment 48 or 49, wherein the modified LNA nucleoside is cET with the following 2′-4′ bridge —O—CH(CH₃)—.
53. The nucleic acid molecule of embodiment 52, wherein the cET is (S)cET, i.e. 6′(S)methyl-beta-D-oxy-LNA.
54. The nucleic acid molecule of embodiment 48 or 49, wherein the LNA is ENA, with the following 2′-4′ bridge —O—CH₂—CH₂—.
55. The nucleic acid molecule of any one of embodiments 24 to 54, wherein the nucleic acid molecule comprises at least one modified internucleoside linkage.
56. The nucleic acid molecule of embodiment 55, wherein the modified internucleoside linkage is nuclease resistant.
57. The nucleic acid molecule of embodiment 55 or 56, wherein the modified internucleoside linkages is a phosphorothioate internucleoside linkages.
58. The nucleic acid molecule any one of embodiments 24 to 57, wherein the nucleic acid molecule is a double stranded siRNA oligonucleotide capable of interacting with the RISC complex.
59. The nucleic acid molecule any one of embodiments 24 to 57, wherein the nucleic acid molecule is an antisense oligonucleotide capable of recruiting RNase H.
60. The antisense oligonucleotide of embodiment 59, wherein the antisense oligonucleotide or the contiguous nucleotide sequence is a gapmer.
61. The antisense oligonucleotide of embodiment 60, wherein the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and defines the 5′ and 3′ end of the F and F′ region, and G is a region between 6 and 16 nucleosides which are capable of recruiting RNaseH.
62. The antisense oligonucleotide of embodiment 61, wherein the 2′ sugar modified nucleoside independently is selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.
63. The antisense oligonucleotide of embodiment 61 or 62, wherein one or more of the 2′ sugar modified nucleosides in region F and F′ is a LNA nucleoside
64. The antisense oligonucleotide of embodiment 63, wherein all the 2′ sugar modified nucleosides in region F and F′ are LNA nucleosides.
65. The oligonucleotide of embodiment 62 to 64, wherein the LNA nucleoside is selected from beta-D-oxy-LNA, alpha-L-oxy-LNA, beta-D-amino-LNA, alpha-L-amino-LNA, beta-D-thio-LNA, alpha-L-thio-LNA, (S)cET, (R)cET beta-D-ENA and alpha-L-ENA.
66. The antisense oligonucleotide of embodiment 62 to 65, wherein region F and F′ consist of identical LNA nucleosides.
67. The antisense oligonucleotide of embodiment 62 to 66, wherein all the 2′ sugar modified nucleosides in region F and F′ are oxy-LNA nucleosides.
68. The antisense oligonucleotide of any one of embodiments 61 to 67, wherein the nucleosides in region G is DNA and/or alpha-L-LNA nucleosides.
69. The antisense oligonucleotide of embodiment 68, wherein region G consists of at least 75% DNA nucleosides.
70. The antisense oligonucleotide of embodiment 69, where all the nucleosides in region G are DNA nucleosides.
71. A conjugate compound comprising a nucleic acid molecule according to any one of embodiments 24 to 59 or an antisense oligonucleotide according to any one of embodiments 60 to 70, and at least one conjugate moiety covalently attached to said antisense oligonucleotide.
72. The conjugate compound of embodiment 71, wherein the nucleic acid molecule is a double stranded siRNA and the conjugate moiety is covalently attached to the sense strand of the siRNA.
73. The conjugate compound of embodiment 71 or 72, wherein the conjugate moiety is selected from carbohydrates, cell surface receptor ligands, drug substances, hormones, lipophilic substances, polymers, proteins, peptides, toxins, vitamins, viral proteins or combinations thereof.
74. The conjugate compound of any one of embodiments 71 to 73, wherein the conjugate moiety is capable of binding to the asialoglycoprotein receptor.
75. The conjugate compound of embodiment 74, wherein the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine.
76. The conjugate compound of embodiment 75, wherein the asialoglycoprotein receptor targeting moiety is N-acetylgalactosamine (GalNAc).
77. The conjugate compound of embodiment 75 or 76, wherein the conjugate moiety is mono-valent, di-valent, tri-valent or tetra-valent with respect to asialoglycoprotein receptor targeting moieties.
78. The conjugate compound of embodiment 77, wherein the conjugate moiety consists of two to four terminal GalNAc moieties and a spacer linking each GalNAc moiety to a brancher molecule that can be conjugated to the antisense compound.
79. The conjugate compound of embodiment 78, wherein the spacer is a PEG spacer.
80. The conjugate compound of embodiment 74 to 79, wherein the conjugate moiety is a tri-valent N-acetylgalactosamine (GalNAc) moiety.
81. The conjugate compound of embodiment 74 to 80, wherein the conjugate moiety is selected from one of the trivalent GalNAc moieties in FIG. 1.
82. The conjugate compound of embodiment 81, wherein the conjugate moiety is the trivalent GalNAc moiety in FIG. 1D.
83. The conjugate compound of embodiment 71-82, comprising a linker which is positioned between the nucleic acid molecule or the antisense oligonucleotide and the conjugate moiety.
84. The conjugate compound of embodiment 83, wherein the linker is a physiologically labile linker.
85. The conjugate compound of embodiment 84, wherein the physiologically labile linker is nuclease susceptible linker.
86. The oligonucleotide conjugate of embodiment 84 or 85, wherein the physiologically labile linker is composed of 2 to 5 consecutive phosphodiester linkages.
87. The conjugate compound of embodiment 74-86, which display improved cellular distribution between liver vs. kidney or improved cellular uptake into the liver of the conjugate compound as compared to an unconjugated nucleic acid molecule or antisense oligonucleotide.
88. A pharmaceutical composition comprising a nucleic acid molecule according to any one of embodiments 24 to 59 or an antisense oligonucleotide according to any one of embodiments 60 to 70, a conjugate compound of embodiment 71 to 87 or acceptable salts thereof and a pharmaceutically acceptable diluent, carrier, salt and/or adjuvant.
89. A method for identifying a compound that prevents, ameliorates and/or inhibits a hepatitis B virus (HBV) infection, comprising:
   a. contacting a test compound with
   b. FUBP1 polypeptide; or
   c. a cell expressing FUBP1;
   d. measuring the expression and/or activity of FUBP1 in the presence and absence of said test compound; and
   e. identifying a compound that reduces the expression and/or activity FUBP1 and reduces cccDNA.
90. An in vivo or in vitro method for modulating FUBP1 expression in a target cell which is expressing FUBP1, said method comprising administering the nucleic acid molecule of any one of embodiments 24 to 59 or an antisense oligonucleotide according to any one of embodiments 60 to 70, a conjugate compound of embodiment 71 to 87 or the pharmaceutical composition of embodiment 88 in an effective amount to said cell.
91. The method of embodiments 90, wherein the FUBP1 expression is reduced by at least 50%, or at least 60%, or at least 70%, or at least 80%, or at least 90%, or at least 95% in the target cell compared to the level without any treatment or treated with a control.
92. The method of embodiments 90, wherein the target cell is infected with HBV and the cccDNA in an HBV infected cell is reduced by at least 50%, or at least 60%, or at least 70%, or at least 80%, or at least 90%, or at least 95% in the HBV infected target cell compared to the level without any treatment or treated with a control.
93. A method for treating or preventing a disease comprising administering a therapeutically or prophylactically effective amount of the nucleic acid molecule any one of embodiments 24 to 59 or an antisense oligonucleotide according to any one of embodiments 60 to 70, a conjugate compound of embodiment 71 to 87 or the pharmaceutical composition of embodiment 88 to a subject suffering from or susceptible to the disease.
94. The nucleic acid molecule of any one of embodiments 24 to 59 or the antisense oligonucleotide according to any one of embodiments 60 to 70, or the conjugate compound of any one of embodiments 71 to 87 or the pharmaceutical composition of embodiment 88, for use as a medicament for treatment or prevention of a disease in a subject.
95. Use of the nucleic acid molecule any one of embodiments 24 to 59 or the antisense oligonucleotide according to any one of embodiments 60 to 70, or the conjugate compound of any one of embodiments 71 to 87 for the preparation of a medicament for treatment or prevention of a disease in a subject.
96. The method, the nucleic acid molecule, the antisense oligonucleotide, the conjugate or the use of embodiments 93-95 wherein the subject is a mammal.
97. The method, the nucleic acid molecule, the antisense oligonucleotide, the conjugate, or the use of embodiment 96, wherein the mammal is human.

### EXAMPLES

The examples illustrate the invention.

### Material and Methods

### HepG2-NTCP Cell Line and HBV Infection

HepG2-NTCP cells (Urban et al. gastroenterology 2014, DOI:10.1053/j.gastro.2013.12.024) were cultured at 37° C. in a humidified atmosphere with 5% CO2 in complete proliferation medium consisting of DMEM+GlutaMAX-I (Gibco #31966-021), 5% HI FCII (Gibco), 1× Pen/Strep (Gibco, #15140), for 2 weeks. For HBV infection HepG2-NTCP cells were trypsinized and resuspended in Infection Medium (complete proliferation medium supplemented with 2.5%

DMSO) and seeded into a 12 well plate at 400 000 cells/well. After 3 to 4 days, cells were then inoculated with HBV once they reached between 80% to 90% confluency (referred to as day 0) using 500ul Infection Medium plus 4% PEG 8000 and HBV (MOI100) per well. After 16 h the cells were washed 3 times with phosphate-buffered saline and the medium was replaced every 3-4 days with 1 ml Modified Infection Medium (complete proliferation medium supplemented with 3.5% DMSO) per well. HBV genotype D was derived from HepAD38 cell culture supernatant and concentrated using PEG precipitation (Ladner et al 1997 Antimicrobial Agents and Chemotherapy 41(8) 171-1720).

HeLa Cell lines

HeLa cell line was purchased from European Collection of Authenticated Cell Cultures (ECACC, #93021013) and maintained as recommended by the supplier in a humidified incubator at 37° C. with 5% CO2. For assays, 2,500 cells/well were seeded in a 96 multi well plate in Eagle's Minimum Essential Medium (Sigma, M2279) with 10% fetal bovine serum (FBS), 2 mM Glutamin AQ, 1% NEAA, 25 μg/ml Gentamicin.

### Primary Mouse Hepatocytes (PMH)

Primary mouse hepatocytes were isolated from livers of C57BL/6J mice anesthetized with Pentobarbital after a 2 step perfusion protocol according to the literature (Berry and Friend, 1969, J. Cell Biol; Paterna et al., 1998, Toxicol. Appl. Pharmacol.). The first step was 5 min with HBSS+15 mM HEPES+0.4 mM EGTA followed by 12 min HBSS+20 mM NaHCO3+0.04% BSA (Sigma #A7979)+4 mM CaCL2 (Sigma #21115)+0.2 mg/ml Collagenase Type 2 (Worthington #4176). The Hepatocytes were captured in 5 ml cold Williams medium E (WME) (Sigma #W1878, complemented with 1× Pen/Strep/Glutamine, 10% (v/v) FBS (ATCC #30-2030)) on ice.

The crude cell suspension was filtered through a 70 μm followed by a 40 μm cell strainer (Falcon #352350 and #352340), filled up to 25 ml with WME and centrifuged at room temperature for 5 min at 50×g to pellet the hepatocytes. The supernatant was removed and the hepatocytes were resuspended in 25 ml WME. After adding 25 ml 90% Percoll solution (Sigma #P4937; pH=8.5-9.5) and centrifugation for 10 min at 25 ° C., 50×g the supernatant and floating cells were removed. To remove the remaining Percoll the pellet was resuspended again in 50 mL WME medium, centrifuged 3 min, 25 ° C. at 50×g and the supernatant discarded. The cell pellet was resuspended in 20 mL WME and cell number and viability determined (Invitrogen, Cellcount) and diluted to 250,000 cells/ml. 25,000 cells/well were seeded on collagen-coated 96-well plates (PD Biocoat Collagen I #356407) and incubated at 37 ° C., 5% CO2. 24 h after seeding the oligonucleotides were added in the desired concentration and the cells were incubated at 37 ° C., 5% CO2 for 72 hours.

### HBV Infected PHH Cells

Fresh primary human hepatocytes (PHH) were provided by PhoenixBio, Higashi-Hiroshima City, Japan (Cat #PXB-cells) in 24-well plate format. Upon arrival the PHH were infected with an MOI of 25 GE using HepG2 2.2.15-derived HBV by incubating the PHH cells with HBV in 4% (v/v) PEG in PHH medium for 16 hours. The cells were then washed thrice with PBS and cultured in a humidified atmosphere with 5% CO2 in fresh PHH medium consisting of DMEM (GIBCO, Cat #21885) supplemented with 10% (v/v) heat-inactivated fetal bovine serum (GIBCO, Cat #10082), 2% (v/v) DMSO, 1% (v/v) Penicillin/Streptomycin (GIBCO, Cat #15140-148), 20 mM HEPES (GIBCO, Cat #15630-080), 44 mM NaHCO3 (Wako, Cat #195-14515), 15 ug/ml L-proline (MP-Biomedicals, Cat #0219472825), 0.25 ug/ml Insulin (Sigma, Cat #I1882), 50 nM Dexamethasone (Sigma, Cat# D8893), 5 ng/ml EGF (Sigma, Cat# E9644), and 0.1 mM L-Ascorbic acid 2-phosphate (Wako, Cat #013-12061).

siRNA Sequences and Compounds

TABLE 4
Human FUBP1 sequences targeted by individual
FUBP1 siRNA molecules

| SEQ | | Position | | | |
|---|---|---|---|---|---|
| ID | FUBP1 target | on SEQ | | Individual | Dharmacon |
| NO: | sequence | ID NO: 1 | Exon | SIRNA | catalog No. |
| 21 | GACAAACCUCUUAGGAUUA | 14200-14218 | 9 | FU2 | J-011548-06 |
| 22 | GAGAAGUUCGGAAUGAGUA | 14413-14431 | 10 | FU4 | J-011548-08 |
| 23 | GAAAGGAUAGCACAAAUAA | 14966-14984 | 12 | FU1 | J-011548-05 |
| 24 | AAUAAGAAGUGGACAAUAC | 30344-30362 | 20 | FU3 | J-011548-07 |

The pool of siRNA (ON-TARGETplus SMART pool siRNA Cat. No. L-011548-00-0005, Dharmacon) contains the four individual siRNA molecules listed in table 4 and are available.

### Single Stranded Antisense Oligonucleotides

TABLE 4A
List of oligonucleotide motif sequences (indicated by SEQ ID NO),
designs of these, as well as specific oligonucleotide compounds
(indicated by CMP ID NO) designed based on the motif sequence.

| | | | | | Start |
|---|---|---|---|---|---|
| SEQ | | | | CMP | position |
| ID | | | Oligonucleotide | ID | on SEQ |
| NO | Motif sequence | Design | compound | NO | ID NO: 1 |
| 34 | ataaccatagtcatttga | 3-12-3 | ATAaccatagtcattTGA | 34_1 | 9138 |
| 35 | cccataaccatagtcat | 3-12-2 | CCCataaccatagtcAT | 35_1 | 9142 |
| 36 | gagccatctacacataaa | 2-12-4 | GAgccatctacacaTAAA | 36_1 | 10016 |
| 37 | caagagccatctacacat | 3-13-2 | CAAgagccatctacacAT | 37_1 | 10019 |
| 38 | tgtccatttaagaatcca | 4-12-2 | TGTCcatttaagaatcCA | 38_1 | 10144 |
| 39 | ttgtgtccatttaagaat | 4-12-2 | TTGTgtccatttaagaAT | 39_1 | 10147 |
| 40 | tacctttgctgctgat | 3-11-2 | TACctttgctgctgAT | 40_1 | 11472 |
| 41 | gactatacctttgctgc | 2-13-2 | GActatacctttgctGC | 41_1 | 11476 |
| 42 | actttgtattcttctgtcat | 1-16-3 | ActttgtattcttctgtCAT | 42_1 | 12020 |
| 43 | actttgtattcttctgtc | 2-14-2 | ACtttgtattcttctgTC | 43_1 | 12022 |
| 44 | gattcaggtgttccagtta | 1-16-2 | GattcaggtgttccagtTA | 44_1 | 12393 |
| 45 | ttcaaacttactggaca | 3-11-3 | TTCaaacttactggACA | 45_1 | 12412 |
| 46 | tacatctatcccttcat | 2-12-3 | TAcatctatcccttCAT | 46_1 | 14452 |
| 47 | ttacatctatcccttc | 2-10-4 | TTacatctatccCTTC | 47_1 | 14454 |
| 48 | cttcctattacaatgccaac | 1-17-2 | CttcctattacaatgccaAC | 48_1 | 14776 |
| 49 | atttcttcctattacaatg | 2-14-3 | ATttcttcctattacaATG | 49_1 | 14781 |
| 50 | ccatttottcctattacaa | 3-14-2 | CCAtttcttcctattacAA | 50_1 | 14783 |
| 51 | gtttaaaatacattgcc | 2-11-4 | GTttaaaatacatTGCC | 51_1 | 15537 |
| 52 | gagtttaaaatacattgcc | 2-14-3 | GAgtttaaaatacattGCC | 52_1 | 15537 |
| 53 | gctttttatggtttcacc | 1-15-2 | GctttttatggtttcaCC | 53_1 | 16183 |
| 54 | atgctttttatggtttcacc | 1-17-2 | AtgctttttatggtttcaCC | 54_1 | 16183 |
| 55 | ataatcaacctgtccagct | 1-16-2 | AtaatcaacctgtccagCT | 55_1 | 23804 |
| 56 | ataatcaacctgtccagc | 1-15-2 | AtaatcaacctgtccaGC | 56_1 | 23805 |
| 57 | ataatcaacctgtccag | 1-12-4 | AtaatcaacctgtCCAG | 57_1 | 23806 |
| 58 | tataatcaacctgtccag | 2-13-3 | TAtaatcaacctgtcCAG | 58_1 | 23806 |
| 59 | gtataatcaacctgtccag | 1-15-3 | GtataatcaacctgtcCAG | 59_1 | 23806 |
| 60 | ataatcaacctgtcca | 1-11-4 | AtaatcaacctgTCCA | 60_1 | 23807 |
| 61 | tataatcaacctgtcca | 1-12-4 | TataatcaacctgTCCA | 61_1 | 23807 |
| 62 | gtataatcaacctgtcca | 1-14-3 | GtataatcaacctgtCCA | 62_1 | 23807 |
| 63 | gtataatcaacctgtcc | 3-12-2 | GTAtaatcaacctgtCC | 63_1 | 23808 |
| 64 | tataatcaacctgtcc | 2-10-4 | TAtaatcaacctGTCC | 64_1 | 23808 |
| 65 | cccattttcttgtagta | 2-13-2 | CCcattttcttgtagTA | 65_1 | 23841 |
| 66 | acccattttcttgtagta | 1-15-2 | AcccattttcttgtagTA | 66_1 | 23841 |
| 67 | tacccattttcttgtagta | 1-16-2 | TacccattttcttgtagTA | 67_1 | 23841 |
| 68 | atacccattttcttgtagta | 1-17-2 | AtacccattttcttgtagTA | 68_1 | 23841 |
| 69 | atacccattttcttgtag | 1-14-3 | AtacccattttcttgTAG | 69_1 | 23843 |
| 70 | catacccattttcttgtag | 2-15-2 | CAtacccattttcttgtAG | 70_1 | 23843 |
| 71 | catacccattttcttgta | 2-14-2 | CAtacccattttcttgTA | 71_1 | 23844 |
| 72 | taaaactaattcaadaat | 2-14-2 | TGaaactaattcadaaGT | 72_1 | 30264 |
| 73 | aaatggagctaattcaggag | 3-14-3 | AAAtggagctaattcagGAG | 73_1 | 30265 |
| 74 | ttgtccacttcttattatt | 1-14-4 | TtgtccacttcttatTATT | 74_1 | 30341 |
| 75 | ccccacacaatgaagcaa | 2-14-2 | CCccacacaatgaagcAA | 75_1 | 30368 |
| 76 | ttcatcaagtcgtctgcat | 1-16-2 | TtcatcaagtcgtctgcAT | 76_1 | 30412 |
| 77 | gatcttcatcaagtcgtc | 1-15-2 | GatcttcatcaagtcgTC | 77_1 | 30417 |
| 78 | atattaacctcctatcagt | 1-15-3 | AtattaacctcctatcAGT | 78_1 | 30511 |
| 79 | aatattaacctcctatcag | 3-13-3 | AATattaacctcctatCAG | 79_1 | 30512 |
| 80 | atttatatcacaaagcatc | 2-13-4 | ATttatatcacaaagCATC | 80_1 | 30616 |
| 81 | aagtacatttatatcaca | 4-12-2 | AAGTacatttatatcaCA | 81_1 | 30623 |
| 82 | catttattgtaaagcacaaa | 2-14-4 | CAtttattgtaaagcaCAAA | 82_1 | 30675 |
| 83 | atcatttattgtaaagca | 2-12-4 | ATcatttattgtaaAGCA | 83_1 | 30679 |

Motif sequences represent the contiguous sequence of nucleobases present in the oligonucleotide.

Designs refer to the gapmer design, F-G-F′, where each number represents the number of consecutive modified nucleosides, e.g. 2′ modified nucleosides (first number=5′ flank), followed by the number of DNA nucleosides (second number=gap region), followed by the number of modified nucleosides, e.g. 2′ modified nucleosides (third number=3′ flank), optionally preceded by or followed by further repeated regions of DNA and LNA, which are not necessarily part of the contiguous sequence that is complementary to the target nucleic acid.

Oligonucleotide compounds represent specific designs of a motif sequence. Capital letters represent beta-D-oxy LNA nucleosides, lowercase letters represent DNA nucleosides, all LNA C are 5-methyl cytosine, and 5-methyl DNA cytosines are presented by “e”, all internucleoside linkages are phosphorothioate internucleoside linkages.

siRNA Transfection and Treatment Protocol for HBV Infected Cells

siRNA treatments were performed in HepG2-NTCP or PHH. Cells were transfected at day 4 and day 6 post infection with 100 ul/well transfection mixture containing 25 nM of either scramble SIRNA (ON-TARGETplus Non-targeting siRNA, D-001810-01, Dharmacon, CO, USA) , FUBP1 pool or single siRNA (SMARTpool: ON-TARGETplus human FUBP1 siRNA, L-011548-00, Dharmacon, CO, USA) or no siRNA (OptiMEM) (0.5 ul/well) diluted in OptiMEM (50 ul/well, Thermo Fisher Scientific Reduced Serum media), and mixed for 5 minutes prior to transfection with Lipofectamine® RNAiMAX Transfection Reagent (3 ul/well) (Thermofisher Scientific catalog No. 13778) diluted in OptiMEM (50 ul/well).

For PHH southern blot, cells were also treated with an HBV-specific siRNA (HBx siRNA) used as negative control (2 pmol). The HBV-specific siRNA was custom made (GE Life science) (Sense: 5′-GCACUUCGCUUCACCUCUG-3′ SEQ ID NO: 84) in addition to a second negative control (sicontrol) the Silencer® Negative Control #1 siRNA (Thermo Fisher Scientific). The transfection mixture was added to the cells with 1 ml of DMEM without DMSO and Pen/Strep for 24 h, then medium was replaced with HepG2 NTCP and PHH cell culture medium respectively.

The cells were harvested at day 8 post infection (after 4 days of siRNA treatment) and stored at −80 ° C. for analysis of FUBP1 mRNA knockdown and the quantification of viral parameters.

### Southern Blot Protocol

For Southern blot, cccDNA purification from HBV infected PHH was conducted using an adapted Hirt extraction protocol previously described in Hirt B 1967 J Mol Biol 26:365-369; and Cai D 2013 Methods Mol Biol. 1030:151-61. Briefly, cells were lysed with HIRT lysis buffer (10 mM Tris pH8, 10 mM EDTA, 0.7% SDS, 2 ul Ambion RNase Mix/ml buffer) to disrupt lipid membranes and viral capsids in order to release all the viral nucleic acids. RNA was removed by RNAse treatment before precipitating down with NaCl (5 M, overnight at 4° C.) the high-molecular-weight cellular chromatin, proteins and protein-associated DNA including the HBV DNA replicative intermediates with viral polymerase covalently attached. After the overnight high-salt protein precipitation, viral DNA (enriched cccDNA) was extracted from the clarified lysates by direct phenol extraction.

DNA was then quantified and adjusted to load on an agarose gel (1.8% in TAE buffer) 15 μg of each samples in 15 μl per well. DNA ladder (NEB Quick-Load 1 kb), and recombinant HBV were also loaded and the gel was run at 50V for 3 h 30 min.

For the Southern Blot , the gel was submerged in denaturing buffer (0.5 M NaOH (50 ml), 1.5 M NaCl (150 ml)) and rocked for 30 minutes at room temperature to separate the double-stranded DNA into single DNA strands for later hybridization to the probe. The transfer of DNA from the agarose gel to Hybond-XL membrane (Cat. No: RPN2020S, GE Healthcare) was done using Whatman Nytran SuPerCharge (SPC) TurboBlotter Kit (Cat. No: WHA10416328, Sigma). After transfer, the membrane was cross linked in a UV crosslinker chamber with UV energy dosage at 1800 J. A DIG-labelled RNA probe was generated using HBV plasmid DNA (HBV Genotype D cloned into a pGEM3Z plasmid) and the following primers:
Forward Primer HBVT7+Fwd 5′-taatacgactcactatagggtttttcacctctgcctaatcatc-3′ (SEQ ID NO: 89)
Reverse Primer HBVRev 5′-cctctagagcggccgcaaaaagttgcatggtgctggt-3′ (SEQ ID NO: 90) with the DIG RNA Labeling Kit ((SP6/T7) Cat no 11175025910, Roche).

The membrane was then hybridized with the DIG-labelled probe in DIG Easy Hyb buffer (Cat. No: 11603558001, Roche) at 50° C. overnight while rotating in the hybridization oven. The membrane was then washed and incubated with CDP-Star with NitroBlock minutes before exposing to film. Detection of the signal was done with Fusion Fx (VILBER).

### HBV Antigen Measurements

To evaluate the impact on HBV antigen expression and secretion, supernatants from the cell cultures can be collected. The HBV propagation parameters, HBsAg and HBeAg levels, are measured using CLIA ELISA Kits (Autobio Diagnostic #CL0310-2, #CL0312-2), according to the manufacturer's protocol. Briefly, 25 μL of supernatant per well is transferred to the respective antibody coated microtiter plate and 25 μL of enzyme conjugate reagent is added. The plate is incubated for 60min on a shaker at room temperature before the wells are washed five times with washing buffer using an automatic washer. 25 μL of substrate A and B were added to each well. The plates are incubated on a shaker for 10 min at room temperature before luminescence is measured using an Envision luminescence reader (Perkin Elmer).

Real-Time PCR for Intracellular HBV pgRNA and FUBP1 RNA

FUBP1 RNA and HBV pgRNA were quantified by RTqPCR using SYBR green. Results were normalized over the human Gus B endogenous control. The mRNA expression was analysed using the comparative cycle threshold 2-ΔΔCt method normalized to the reference gene Gus B and to non-treated cells. Primers used for FUBP1 RNA and HBV pgRNA quantification are listed in table 5. RTqPCR conditions were as follow: 48° C., 15 min (RT step); 95° C., 10 min; 40 cycles at 95° C., 15 sec and 60° C., 1 min.

TABLE 5
FUBP1 and HBV pgRNA qPCR primers

| Parameter | | Primer Sequence | SEQ ID |
|---|---|---|---|
| FUBP1: | Fwd | TGTCCAGTCAGCAAAACGGTT | 25 |
| | Rev | GAACTGCATTTCCCGGTCCAT | 26 |
| HBV pgRNA | Fwd | GGAGTGTGGATTCGCACTCCT | 27 |
| (modified from | Rev | AGATTGAGATCTTCTGCGAC | 28 |
| Volz et al. | probe | [6FAM]-AGGCAGGTCCCCTA | 29 |
| Gastroenterology | | GAAGAAGAACTCC-[BHQ1] | |
| 2007) | | | |

Housekeeping gene primers Gus B: Hs99999908_m1 (Thermo Fisher Scientific)

HBV DNA and cccDNA Taqman qPCR

DNA was extracted from HBV infected cells (HepG2 NTCP or PHH) using the MasterPure™ DNA Purification Kit (Epicentre, Madison, Wisconsin USA) protocol. cccDNA levels were determined after digestion with T5 exonuclease (New England Biolabs, MA, USA) using 10 U of T5 for 500 ng of DNA, 1 hour at 37° C. in 20 ul total volume. After digestion, the samples were diluted to 50 ul of which 4 ul were used for the qPCR reaction. Quantitative real-time polymerase chain reaction measurements were performed on the ViiA7 Real-Time PCR System (Life Technologies). qPCR was performed with the Taqman® Fast Advanced Master Mix (Life Technologies, Cat. No 4444557). qPCR conditions were as follow: 50° C., 2 min; 95° C., 10 min; 40 cycles at 95° C., 15 sec and 60° C. , 1 min. cccDNA Primers and probe are shown in table 6.

TABLE 6
cccDNA qPCR primers and probe.

| | | | SEQ ID |
|---|---|---|---|
| Parameter | | Primer Sequence | NO |
| cccDNA | Fwd | 5′-CCGTGTGCACTTCGCTTCA-3′ | 30 |
| | Rev | 5′-GCACAGCTTGGAGGCTTGA-3′ | 31 |
| | Probe | 5′-[6FAM]CATGGAGACCACCGTG | 32 |
| | | AACGCCC[BHQ1]-3′ | |

The cccDNA primer and probes are described in Malmström et al. 2012 PLOS ONE 7(7): e36349.

b-globin was quantified using the HBB TaqMan® Gene Expression assay (ID Hs00758889_s1, Thermofischer scientific) and total HBV DNA with Taqman primers (Pa03453406_s1, Thermofischer).

### Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 umol scale. At the end of the synthesis, the oligonucleotides are cleaved from the solid support using aqueous ammonia for 5-16hours at 60° C. The oligonucleotides are purified by reverse phase HPLC (RP-HPLC) or by solid phase extractions and characterized by UPLC, and the molecular mass is further confirmed by ESI-MS.

### Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle, a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

### Purification by RP-HPLC:

The crude compounds are purified by preparative RP-HPLC on a Phenomenex Jupiter C18 10μ 150×10 mm column. 0.1 M ammonium acetate pH 8 and acetonitrile is used as buffers at a flow rate of 5 mL/min. The collected fractions are lyophilized to give the purified compound typically as a white solid.

### Abbreviations:

DCI: 4,5-Dicyanoimidazole
DCM: Dichloromethane
DMF: Dimethylformamide
DMT: 4,4′-Dimethoxytrityl
THF: Tetrahydrofurane
Bz: Benzoyl
Ibu: Isobutyryl
RP-HPLC: Reverse phase high performance liquid chromatography

### Tₘ Assay:

Oligonucleotide and RNA target (phosphate linked, PO) duplexes are diluted to 3 mM in 500 ml RNase-free water and mixed with 500 ml 2× Tₘ-buffer (200 mM NaCl, 0. 2mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

### Example 1: Chromatin Immunoprecipitation (ChiP) of FUBP1 in HBV Infected HepG2-NTCP

In this experiment, it was investigated whether FUBP1 binds to cccDNA. In brief, chromatin fragments from HBV infected cells were immunoprecipitated with a FUBP1-specific antibody and it was analysed whether cccDNA was pulled down together with the FUBP1 Ab.

HBV infected HepG2-NTCP cells (see materials and method section) were crossed link with 1% formaldehyde in Williams medium without serum or antibiotics for 10 minutes and the reaction was stopped by adding 0.125 M glycine in PBS 1×, 5 min at room temperature. Cells were washed 2 times with cold PBS and harvested with a cell scraper. The pelleted cells were collected after centrifugation at 4500 rpm for 10 min at 4° C. in the presence of 1 ml of PBS 1× with PIC 1× and PMSF 1 mM and stored at −80° C. before proceeding to the chromatin immunoprecipitation. Nuclear extraction was performed using a dounce (tight pestle) after 30 minutes of incubation on ice in Nuclei Lysis buffer (PIPES 5 mM, KCl 85 mM, NP-40 0.5%, PMSF 1 mM and PIC 1×)

Isolated cross-linked nuclei were sheared by sonication in a 1% SDS sonication buffer to generate cellular chromatin fragments of 200-500 bp. Chromatin amounts from 2×10⁶ cells was separated, 1/10 of this was saved to serve as reference input sample and the reminder was subjected to immunoprecipitation with antibody for 14-16 hours at 4° C. (ChIP is further described in Pollicino et al 2006 Gastroenterologyl30:823-837). The antibody used for the immunoprecipitation was 5 μg FUBP1-specific Ab (C15410233 Diagenode). After the reverse cross-linking step, the precipitated chromatin sample and the input sample were subjected to Phenol: Chlorophorm: Isoamyl Alcohol (25:24:1) DNA extraction and cccDNA was then quantified by qPCR (see materials and method section on HBV cccDNA quantification starting with T5 exonuclease treatment and with the change that the cccDNA is not normalized to b-globulin (not precipitated by the antibody), but is expressed as the percentage of cccDNA in the input sample).

cccDNA amount in the samples precipitated with and without FUBP Ab is expressed as % of cccDNA in the input sample (non-ChIP treated sample). The results are shown in table 7. The values represent the mean of three independent experiments.

TABLE 7
cccDNA immunoprecipitated with a FUBP1-
specific antibody or control.

% cccDNA of input sample

| | Mean | SD |
|---|---|---|
| NoAb | 0.0043 | 0.0025 |
| FUBP1 | 0.1528 | 0.0364 |

The ChIP experiment showed significant enrichment of FUBP1 protein bound to cccDNA compared to negative control (NoAb).

### Example 2: Effect of siRNAs Targeting FUBP1 on HBV Parameters in HBV Infected HepG2-NTCP Cells

In the following experiment, the effect of FUBP1 knock-down on the HBV parameters, HBV pgRNA and cccDNA, was tested.

HBV infected HepG2-NTCP cells were treated with the pool of siRNAs from Dharmacon (L-011548-00) as described in the Materials and Method section “siRNA transfection and treatment protocol for HBV infected cells”.

Following the 4 days-treatment, FUBP1 mRNA, cccDNA and pgRNA were measured by qPCR. FUBP1 mRNA and pgRNA RT-qPCR was described in the materials and method section “Real-time PCR for intracellular HBV pgRNA and FUBP1 RNA”. For cccDNA absolute quantification in HepG2 NTCP cells, b-globin was measured using 500ng non-digested DNA in a total volume of 50 ul of which 4 ul were used in the qPCR and used for normalization. Commercial genomic DNA was used for the standard curve for b-globin and an HBV plasmid DNA (HBV Genotype D cloned into a pGEM3Z plasmid) was used for cccDNA standard curve. The cccDNA primers used were those described in the Material and Method section “HBV DNA and cccDNA taqman qPCR”. The results are shown in table 8.

TABLE 8
effect on HBV parameters following
knockdown of FUBP1 with pool of siRNA

| | FUBP1 | | HBV | | cccDNA/ | |
|---|---|---|---|---|---|---|
| | mRNA* | | pgRNA* | | b-globin | |
| | (n = 3) | | (n = 4) | | (n = 4)\hat{ } | |
| | Mean | SD | Mean | SD | Mean | SD |
| No treatment | 1.22 | 0.80 | 2.85 | 1.46 | 0.08 | 0.07 |
| siRNA control | 1.13 | 0.11 | 2.82 | 0.55 | 0.10 | 0.08 |
| FUBP1 siRNA | 0.25 | 0.27 | 1.12 | 0.55 | 0.03 | 0.04 |

*FUBP1 and HBV pgRNA were normalized to housekeeping gene Gus B.
\hat{ }siRNA control was n = 3

From this it can be seen that the FUBP1 siRNA pool is capable of reducing FUBP1 mRNA as well as HBV pgRNA quite efficiently. A trend showing reduction of cccDNA is also observed, despite the short treatment time.

### Example 3: Effect of siRNAs Targeting FUBP1 on HBV Parameters in HBV Infected PHH Cells

In the following experiment, the effect of FUBP1 knock-down on the HBV parameters, HBV pgRNA, cccDNA and HBV DNA, was tested in HBV infected primary human hepatocytes (PHH),

HBV infected PHH cells were treated with the pool of siRNAs used in Example 2 (Dharmacon L-011548-00) as well as four individual siRNA compounds FU1, FU2, FU3 and FU4 listed in the siRNA sequence and compound section of the materials and methods. The treatment was conducted as described in the materials and method section “siRNA transfection and treatment protocol for HBV infected cells”.

Following the 4 days-treatment, FUBP1 mRNA, cccDNA and pgRNA were measured by qPCR in technical duplicate. FUBP1 mRNA and pgRNA RT-qPCR was described in the Materials and Method section “Real-time PCR for intracellular HBV pgRNA and FUBP1 RNA”. HBV DNA was measured as described in the Materials and Methods section “HBV DNA and cccDNA taqman quantification qPCR” The HBV DNA was normalized to the housekeeping gene b-globin using the indicated Taqman primers. For cccDNA quantification, SYBR green primers for cccDNA and mitochondrial DNA (MitoDNA, housekeeping control) were performed using Fast SYBR™ green master mix (Thermo Fischer) and the following primers:

| | cccDNA: |
|---|---|
| | Fwd |
| (SEQ ID NO: 85) | |
| | 5′-CGTCTGTGCCTTCTCATCTGC-3; |
| | Rev |
| (SEQ ID NO: 86) | |
| | 5′-GCACAGCTTGGAGGCTTGAA-3′ |
| | MitoDNA: |
| | Fwd |
| ((SEQ ID NO: 87) | |
| | 5′-CCGTCTGAACTATCCTGCCC-3′; |
| | Rev |
| (SEQ ID NO: 88) | |
| | 5′-GCCGTAGTCGGTGTACTCGT-3′ |

qPCR conditions: 95° C. 5 min, then 45 cycles of 95° C.—1 sec, 60° C.—35 sec

The results are shown in table 9 as the relative expression to non-treated cells using the comparative cycle threshold 2-ΔΔCt method (i.e. the lower the number the larger the target reduction).

TABLE 9
effect on HBV parameters following
knockdown of FUBP1 with siRNA targeting FUBP1

| | FUBP1 | | HBV | | | | HBV | |
|---|---|---|---|---|---|---|---|---|
| | mRNA^{#} | | pgRNA^{#} | | cccDNA* | | DNA** | |
| | Mean | SD | Mean | SD | Mean | SD | Mean | SD |
| Non-treated | 1.030 | 0.105 | 1.00 | 0.07 | 1.00 | 0.04 | 1.00 | 0.07 |
| FUBP1 | 0.66 | 0.04 | 0.61 | 0.04 | 0.64 | 0.06 | 0.70 | 0.04 |
| siRNA pool | | | | | | | | |
| FU1 | 0.68 | 0.02 | 0.53 | 0.10 | 0.62 | 0.04 | 0.51 | 0.04 |
| FU2 | 0.72 | 0.02 | 0.43 | 0.06 | 0.61 | 0.11 | 0.96 | 0.03 |
| FU3 | 0.60 | 0.01 | 0.45 | 0.02 | 0.70 | 0.03 | 0.94 | 0.03 |
| FU4 | 0.71 | 0.02 | 0.38 | 0.01 | 0.78 | 0.09 | 0.61 | 0.03 |

*cccDNA was normalized to mitochondrial DNA reference gene
**HBV DNA was normalized to b-globin housekeeping gene
^{#}FUBP1 mRNA and HBV pgRNA was normalized to Gus B housekeeping gene.

From this it can be seen that the FUBP1 siRNA pool as well as the individual siRNA compounds are capable of reducing FUBP1 mRNA and cccDNA by 30 to 40%. The HBV pgRNA is reduced even further. HBV DNA reduction is observed for the pool of siRNA's and the individual siRNA's FU1 and FU4. The lack of reduction observed with individual siRNA's FU2 and FU3 may be due to the short treatment times since HBV DNA reduction generally require longer treatment times.

The ability of the FUBP1 siRNA pool to reduce cccDNA was confirmed by Southern blot and compared with an HBV targeting siRNA (HBx) which is known not to reduce cccDNA.

HBV infected PHH cells were treated as described in Materials and Method section “siRNA transfection and treatment protocol for HBV infected cells” and the Southern blot was performed as described in the Materials and Methods section “Southern Blot protocol”.

The Southern blot is shown in FIG. 3 and confirms the qPCR results in this example, since it is clearly seen that the Pool of FUBP1 targeting siRNA's are capable of reducing the cccDNA band whereas the HBV targeting siRNA does (as expected) not result in any reduction of cccDNA when compared to the control.

### Example 4: The Effect of FUBP1 Small Molecule Inhibitors on HBV Parameters in HBV Infected PHH Cells

Several small molecules have been suggested to affect FUBP1 binding to the FUSE element on DNA. In the following we tested the effect of SN-38 (formula X herein) and GSK343 (formula VIII herein) for their ability to affect HBV parameters, HBV pgRNA, cccDNA and HBV DNA as well as FUBP1 mRNA levels.

The treatments were performed in HBV infected PHH cells (See Materials and Methods) with small molecules SN-38 (Toronto research chemical, TRC-S589960) and GSK343 (MedChemExpress, MCE-HY-13500) used at 1 μM. The treatment was performed twice weekly starting day 7 and up to day 25 post infection (i.e. treatment was performed on day 7, 11, 14, 18, 21 and 25 post infection). The cells were harvested at day 12 and day 25 post infection (5 days and 18 days of treatment). Following the 5 days or 18 days treatment, FUBP1 mRNA, cccDNA, pgRNA and HBV DNA were measured by qPCR in technical duplicate. FUBP1 mRNA and pgRNA RT-qPCR was measured as described in the materials and method section “Real-time PCR for intracellular HBV pgRNA and FUBP1 RNA”. HBV DNA and cccDNA was normalized to b-globin housekeeping gene as described in the Material and Methods section “HBV DNA and cccDNA taqman qPCR”.

The results are shown in table 10A and 10B as the relative expression to non-treated cells (i.e. the lower the number the larger the target reduction). The toxicity of the compounds was also tested using a standard CCK8 toxicity assay, and no cell toxicity was observed for the two compounds (data not shown).

TABLE 10A
Effect on HBV parameters after 5 days treatment with FUBP1
small molecule inhibitors in HBV infected PPH.

| | FUBP1 | | HBV | | | | HBV | |
|---|---|---|---|---|---|---|---|---|
| | mRNA* | | pgRNA^{#} | | cccDNA* | | DNA* | |
| | Mean | SD | Mean | SD | Mean | SD | Mean | SD |
| Non-treated | 1.00 | 0.09 | 1.00 | 0.05 | 1.01 | 0.17 | 1.01 | 0.06 |
| SN-38 1 uM | 1.04 | 0.01 | 0.54 | 0.03 | 0.60 | 0.10 | 0.42 | 0.00 |
| GSK343 | 0.90 | 0.01 | 0.99 | 0.02 | 1.18 | 0.22 | 1.05 | 0.07 |
| 1 uM | | | | | | | | |

TABLE 10B
Effect on HBV parameters after 18 days treatment with FUBP1
small molecule inhibitors in HBV infected PPH

| | FUBP1 | | HBV | | | | HBV | |
|---|---|---|---|---|---|---|---|---|
| | mRNA^{#} | | pgRNA^{#} | | cccDNA* | | DNA* | |
| | Mean | SD | Mean | SD | Mean | SD | Mean | SD |
| Non-treated | 1.00 | 0.06 | 1.00 | 0.00 | 1.23 | 0.95 | 1.04 | 0.32 |
| SN-38 1 uM | 0.96 | 0.06 | 0.89 | 0.15 | 0.84 | 0.02 | 0.51 | 0.01 |
| GSK343 | 1.27 | 0.17 | 1.34 | 0.09 | 0.34 | 0.16 | 1.29 | 0.34 |
| 1 uM | | | | | | | | |

*cccDNA HBV DNA was normalized to b-globin housekeeping gene
^{#}FUBP1 mRNA and HBVpgRNA were normalized to Gus B housekeeping gene

From these data it can be seen that the FUBP1 small molecule inhibitors do not affect FUBP1 mRNA, which is expected since they act as antagonists of the FUBP1 protein. SN-38 has a significant effect on all three HBV parameters at day 12 at day 25 the effects on pgRNA and cccDNA seem to be rebounding. GSK343 does not have a marked effect on pgRNA and HBV DNA, the reduction of cccDNA is however more than 65% at day 25, indicating that this compound requires longer exposure to be effective, but then it is very effective on cccDNA. It remains to be shown if an even longer exposure would also lead to effects on pgRNA and HBVDNA.

### Example 5: The Ability of Single Stranded LNA Antisense Oligonucleotides to Reduce FUBP1 In Vitro in Hela Cells

The antisense oligonucleotides listed in table 4a were tested for their ability to reduce FUBP1 in Hela cells.

Hela cells were cultured as described in the Materials and Method section. The cells were incubated for 24 hours before addition of oligonucleotides dissolved in PBS. Final concentration of oligonucleotides was 5 and 25 μM, the final culture volume was 100 μl/well. The cells were harvested 3 days after addition of oligonucleotide compounds. RNA was extracted using RNeasy 96 extraction kit (Qiagen,) and followed by one-step RT-QPCR (Quanta Bioscience, qScript XLT 1-Step RT-qPCR ToughMix) using TaqMan assays for the target genes (FUBP1: Hs.PT.58.26883775 FAM (IDT)) and a house keeping gene (GUSB 4326320E (Thermo Fisher)) according to the manufacturer's protocols.

The relative FUBP1 mRNA expression levels are shown in table 11 as % of average control samples (PBS-treated cells) i.e. the lower the value the larger the inhibition.

TABLE 11
in vitro efficacy of anti-FUBP1 compounds
(single experiment with duplex QPCR). FUBP1
mRNA levels are normalized to GUSB in Hela
cells and shown as % of control (PBS
treated cells).

| | % FUBP1 mRNA | | | | |
|---|---|---|---|---|---|
| CMP | of control | | | | |
| ID | 5 μM | | 25 μM | | |
| NO | Avg | sd | Avg | sd | Compound (CMP) |
| 34_1 | 65.5 | 1.4 | 35.2 | 2.0 | ATAaccatagtcattTGA |
| 35_1 | 58.5 | 0.1 | 27.4 | 0.7 | CCCataaccatagtcAT |
| 36_1 | 81.6 | 0.1 | 57.5 | 4.9 | GAgccatctacacaTAAA |
| 37_1 | 85.7 | 2.0 | 64.6 | 0.9 | CAAgagccatctacacAT |
| 38_1 | 74.8 | 4.1 | 49.9 | 0.3 | TGTCcatttaagaatcCA |
| 39_1 | 76.2 | 0.9 | 61.2 | 1.4 | TTGTgtccatttaagaAT |
| 40_1 | 78.1 | 2.7 | 64.2 | 3.1 | TACctttgctgctgAT |
| 41_1 | 88.2 | 3.9 | 66.0 | 0.8 | GActatacctttgctGC |
| 42_1 | 82.7 | 2.9 | 63.2 | 0.5 | ActttgtattcttctgtCAT |
| 43_1 | 76.3 | 1.4 | 56.3 | 0.8 | ACtttgtattcttctgTC |
| 44_1 | 89.5 | 3.9 | 69.9 | 4.9 | GattcaggtgttccagtTA |
| 45_1 | 87.3 | 4.5 | 66.3 | 5.4 | TTCaaacttactggACA |
| 46_1 | 81.1 | 3.0 | 60.3 | 0.1 | TAcatctatcccttCAT |
| 47_1 | 83.8 | 0.5 | 52.7 | 1.7 | TTacatctatccCTTC |
| 48_1 | 74.9 | 12.7 | 64.1 | 2.7 | CttcctattacaatgccaAC |
| 49_1 | 81.3 | 3.3 | 68.7 | 2.2 | ATttcttcctattacaATG |
| 50_1 | 60.9 | 9.3 | 43.2 | 5.7 | CCAtttcttcctattacAA |
| 51_1 | 69.6 | 3.1 | 50.5 | 2.4 | GTttaaaatacatTGCC |
| 52_1 | 68.5 | 0.9 | 53.3 | 1.0 | GAgtttaaaatacattGCC |
| 53_1 | 69.6 | 1.6 | 46.8 | 1.2 | GctttttatggtttcaCC |
| 54_1 | 77.5 | 1.6 | 65.9 | 3.2 | AtgctttttatggtttcaCC |
| 55_1 | 86.8 | 3.3 | 77.8 | 2.7 | AtaatcaacctgtccagCT |
| 56_1 | 85.0 | 6.1 | 82.6 | 0.0 | AtaatcaacctgtccaGC |
| 57_1 | 84.2 | 0.9 | 71.8 | 1.0 | AtaatcaacctgtCCAG |
| 58_1 | 82.1 | 1.3 | 73.3 | 1.2 | TAtaatcaacctgtcCAG |
| 59_1 | 81.4 | 0.6 | 71.2 | 0.1 | GtataatcaacctgtcCAG |
| 60_1 | 86.2 | 1.6 | 64.8 | 2.1 | AtaatcaacctgTCCA |
| 61_1 | 86.0 | 1.3 | 66.4 | 1.5 | TataatcaacctgTCCA |
| 62_1 | 79.3 | 2.5 | 59.5 | 0.6 | GtataatcaacctgtCCA |
| 64_1 | 88.9 | 7.2 | 56.6 | 1.5 | GTAtaatcaacctgtCC |
| 63_1 | 68.8 | 9.1 | 33.7 | 1.0 | TAtaatcaacctGTCC |
| 65_1 | 63.0 | 1.2 | 37.7 | 1.8 | CCcattttcttgtagTA |
| 66_1 | 87.4 | 1.4 | 67.9 | 1.9 | AcccattttcttgtagTA |
| 67_1 | 88.8 | 3.7 | 81.2 | 1.1 | TacccattttcttgtagTA |
| 68_1 | 94.7 | 3.1 | 84.9 | 0.3 | AtacccattttcttgtagTA |
| 69_1 | 87.1 | 1.8 | 64.6 | 0.1 | AtacccattttcttgTAG |
| 70_1 | 83.6 | 6.8 | 81.3 | 0.7 | CAtacccattttcttgtAG |
| 71_1 | 90.2 | 4.9 | 85.6 | 2.4 | CAtacccattttcttgTA |
| 72_1 | 76.8 | 1.9 | 62.8 | 1.5 | TGgagctaattcaggaGT |
| 73_1 | 82.6 | 1.1 | 66.9 | 0.5 | AAAtggagctaattcagGAG |
| 74_1 | 73.3 | 0.8 | 67.5 | 2.5 | TtgtccacttcttatTATT |
| 75_1 | 75.9 | 1.0 | 47.2 | 0.9 | CCccacacaatgaagcAA |
| 76_1 | 75.2 | 0.1 | 53.0 | 3.4 | TtcatcaagtcgtctgcAT |
| 77_1 | 55.2 | 3.9 | 35.2 | 2.2 | GatcttcatcaagtcgTC |
| 78_1 | 83.5 | 3.6 | 69.2 | 0.5 | AtattaacctcctatcAGT |
| 79_1 | 66.5 | 0.4 | 45.7 | 0.2 | AATattaacctcctatCAG |
| 80_1 | 63.0 | 2.7 | 30.2 | 3.0 | ATttatatcacaaagCATC |
| 81_1 | 61.2 | 1.3 | 36.9 | 2.5 | AAGTacatttatatcaCA |
| 82_1 | 84.6 | 0.9 | 68.5 | 0.5 | CAtttattgtaaagcaCAAA |
| 83_1 | 61.7 | 0.5 | 40.4 | 1.8 | ATcatttattgtaaAGCA |

From these data is can be seen that 80% of the single stranded antisense oligonucleotides tested are capable of reducing FUBP1 by at least 30% at the 25 μM dose.

### Example 6: The ability of Single Stranded LNA Antisense Oligonucleotides to Reduce FUBP1 In Vitro in PMH Cells

The oligonucleotides screened in Example 5 also target mouse FUBP1. To verify that the reduction of FUBP1 observed in the human Hela cells also translates to hepatocytes, which is the cell type to be targeted in the treatment of HBV, the same library was tested in primary mouse hepatocytes (PMH).

The screening in PMH cells was conducted as described in the “Materials and Methods” section under “Primary mouse Hepatocytes” using 5 M and 25 μM oligonucleotide, the final culture volume was 100 μl/well.

RNA isolation and qPCR to measure FUBP1 mRNA expression levels were conducted as described in example 5 using the following mouse specific primers instead Fubp1: Mm.PT.58.7603777 FAM-MGB (IDT), and a house keeping gene (GusB Mm_01197698_m1 VIC-MGB (IDT) (Thermo Fisher).

The relative FUBP1 mRNA expression levels are shown in table 12 as % of average control samples (PBS-treated cells) i.e. the lower the value the larger the inhibition.

TABLE 12
in vitro efficacy of anti-FUBP1 compounds
(single experiment with duplex QPCR). FUBP1
mRNA levels are normalized to GUSB in Hela
cells and shown as % of control (PBS
treated cells).

| | % FUBP1 mRNA | | | | |
|---|---|---|---|---|---|
| CMP | of control | | | | |
| ID | 5 μM | | 25 μM | | |
| NO | Avg | sd | Avg | sd | Compound (CMP) |
| 34_1 | 46.1 | 2.5 | 25.2 | 0.5 | ATAaccatagtcattTGA |
| 35_1 | 48.0 | 0.1 | 29.0 | 1.1 | CCCataaccatagtcAT |
| 36_1 | 32.7 | 0.7 | 52.9 | 1.6 | GAgccatctacacaTAAA |
| 37_1 | 50.0 | 1.4 | 48.2 | 0.4 | CAAgagccatctacacAT |
| 38_1 | 48.4 | 1.6 | 35.4 | 0.7 | TGTCcatttaagaatcCA |
| 39_1 | 41.6 | 0.7 | 41.5 | 1.5 | TTGTgtccatttaagaAT |
| 40_1 | 59.2 | 1.1 | 52.8 | 0.5 | TACctttgctgctgAT |
| 41_1 | 88.5 | 1.1 | 59.6 | 0.9 | GActatacctttgctGC |
| 42_1 | 75.8 | 2.0 | 66.9 | 1.9 | ActttgtattcttctgtCAT |
| 43_1 | 71.0 | 2.1 | 59.4 | 0.5 | ACtttgtattcttctgTC |
| 44_1 | 71.4 | 1.4 | 56.7 | 1.2 | GattcaggtgttccagtTA |
| 45_1 | 60.5 | 1.7 | 41.1 | 1.0 | TTCaaacttactggACA |
| 46_1 | 57.5 | 0.3 | 41.5 | 1.2 | TAcatctatcccttCAT |
| 47_1 | 44.5 | 0.5 | 38.3 | 0.5 | TTacatctatccCTTC |
| 48_1 | 58.7 | 0.1 | 45.2 | 0.3 | CttcctattacaatgccaAC |
| 49_1 | 65.0 | 3.6 | 48.2 | 1.4 | ATttcttcctattacaATG |
| 50_1 | 11.7 | 3.2 | 9.0 | 2.6 | CCAtttcttcctattacAA |
| 51_1 | 64.5 | 0.8 | 48.1 | 0.9 | GTttaaaatacatTGCC |
| 52_1 | 68.1 | 1.2 | 51.9 | 1.5 | GAgtttaaaatacattGCC |
| 53_1 | 46.1 | 1.3 | 33.2 | 0.5 | GctttttatggtttcaCC |
| 54_1 | 62.3 | 1.6 | 46.9 | 2.3 | AtgctttttatggtttcaCC |
| 55_1 | 77.8 | 0.1 | 66.3 | 1.6 | AtaatcaacctgtccagCT |
| 56_1 | 78.0 | 2.9 | 60.4 | 1.7 | AtaatcaacctgtccaGC |
| 57_1 | 51.1 | 1.7 | 29.2 | 0.8 | AtaatcaacctgtCCAG |
| 58_1 | 53.7 | 2.6 | 37.7 | 0.4 | TAtaatcaacctgtcCAG |
| 59_1 | 62.8 | 0.1 | 37.0 | 2.1 | GtataatcaacctgtcCAG |
| 60_1 | 51.6 | 0.3 | 40.3 | 0.8 | AtaatcaacctgTCCA |
| 61_1 | 55.3 | 2.0 | 38.1 | 0.8 | TataatcaacctgTCCA |
| 62_1 | 38.5 | 0.9 | 26.4 | 0.1 | GtataatcaacctgtCCA |
| 64_1 | 58.2 | 2.4 | 39.7 | 0.8 | GTAtaatcaacctgtCC |
| 63_1 | 24.7 | 1.0 | 12.6 | 0.4 | TAtaatcaacctGTCC |
| 65_1 | 29.8 | 0.8 | 19.1 | 0.3 | CCcattttcttgtagTA |
| 66_1 | 71.9 | 0.1 | 54.9 | 0.3 | AcccattttcttgtagTA |
| 67_1 | 77.4 | 1.2 | 75.0 | 0.2 | TacccattttcttgtagTA |
| 68_1 | 80.5 | 4.0 | 84.1 | 1.0 | AtacccattttcttgtagTA |
| 69_1 | 67.4 | 0.9 | 49.2 | 1.1 | AtacccattttcttgTAG |
| 70_1 | 77.7 | 0.1 | 67.7 | 2.8 | CAtacccattttcttgtAG |
| 71_1 | 80.1 | 2.6 | 76.3 | 2.5 | CAtacccattttcttgTA |
| 72_1 | 64.5 | 2.7 | 47.1 | 0.8 | TGgagctaattcaggaGT |
| 73_1 | 59.1 | 0.9 | 47.8 | 0.8 | AAAtggagctaattcagGAG |
| 74_1 | 59.1 | 1.1 | 43.2 | 2.2 | TtgtccacttcttatTATT |
| 75_1 | 45.0 | 0.9 | 50.2 | 0.8 | CCccacacaatgaagcAA |
| 76_1 | 52.9 | 1.2 | 40.7 | 0.6 | TtcatcaagtcgtctgcAT |
| 77_1 | 41.5 | 0.1 | 14.8 | 0.1 | GatcttcatcaagtcgTC |
| 78_1 | 68.4 | 2.9 | 54.4 | 0.8 | AtattaacctcctatcAGT |
| 79_1 | 51.4 | 0.8 | 34.7 | 0.3 | AATattaacctcctatCAG |
| 80_1 | 40.3 | 0.8 | 26.3 | 1.2 | ATttatatcacaaagCATC |
| 81_1 | 36.6 | 1.2 | 24.2 | 1.1 | AAGTacatttatatcaCA |
| 82_1 | 51.3 | 5.7 | 45.9 | 0.9 | CAtttattgtaaagcaCAAA |
| 83_1 | 56.0 | 2.6 | 30.6 | 1.4 | ATcatttattgtaaAGCA |

From these data it can be seen that single stranded LNA gapmer oligonucleotides tested in PMH generally are capable of reducing FUBP1 mRNA in hepatocytes and with a higher efficacy than observed in the Hela cells.

### Example 7: The Effect of a Single Stranded LNA Antisense Oligonucleotide on HBV Parameters in HBV Infected PHH cells

Based on the hepatocyte screening in Example 6 the LNA antisense oligonucleotide with CMP ID NO: 50_1 was selected to confirm that the FUBP1 reduction also resulted in an effect on cccDNA in HBV infected PHH cells.

The HBV infected PPH cells (see Materials and Methods section “HBV infected PHH cells”) were treated twice weekly for 2 weeks (i.e. the treatment was performed on day 4, 8, 11, 15, 18, 22 post infection) with 10 μM of FUBP1 LNA in PHH medium and cells were harvested at day 18 and day 25 post infection (14 and 21 days of treatment respectively) for FUBP1 mRNA knockdown and cccDNA qPCR measurement compared to the non-treated group.

The HBV cccDNA and FUBP1 mRNA reduction was measured at day 14 and day 21 following treatment. The results are shown in table 13

TABLE 13
FUBP1 mRNA and cccDNA reduction following treatment
with a single stranded LNA antisense oligonucleotide.

| | FUBP1 mRNA | | cccDNA | |
|---|---|---|---|---|
| | Mean | SD | Mean | SD |
| D 14 | | | | |
| Non treated | 1.005 | 0.09291573 | 1.054 | 0.36328887 |
| CMP ID NO: 50_1 | 0.395 | 0.0212132 | 0.337 | 0.02969848 |
| D 21 | | | | |
| Non treated | 1.016125 | 0.19331428 | 1.0275 | 0.28483972 |
| CMP ID NO: 50_1 | 0.3975 | 0.02505328 | 0.3945 | 0.0205061 |

From this it can be seen that the oligonucleotide of CMP ID NO: 50_1 can reduce cccDNA by 60%.

In summary the examples of the present application show that a double stranded siRNA molecules, FUBP1 small molecule inhibitors, and single stranded antisense oligonucleotides targeting FUBP1 all are capable of reducing cccDNA in HBV infected cells confirming the relevance of FUBP1 in maintaining cccDNA stability.

1. A FUBP1 inhibitor for use in the treatment and/or prevention of Hepatitis B virus (HBV) infection.

2. The FUBP1 inhibitor for the use of claim 1, wherein the HBV infection is a chronic infection.

3. The FUBP1 inhibitor for the use of claim 1 or 2, wherein the FUBP1 inhibitor is capable of reducing cccDNA and/or pgRNA in an infected cell.

4. The FUBP1 inhibitor for the use of any one of claims 1 to 3, wherein the inhibitor prevents or reduces the FUBP1/FUSE interaction.

5. The FUBP1 inhibitor for the use of any one of claims 1 to 4, wherein the inhibitor is selected from the compounds of Formula VII, IX or X

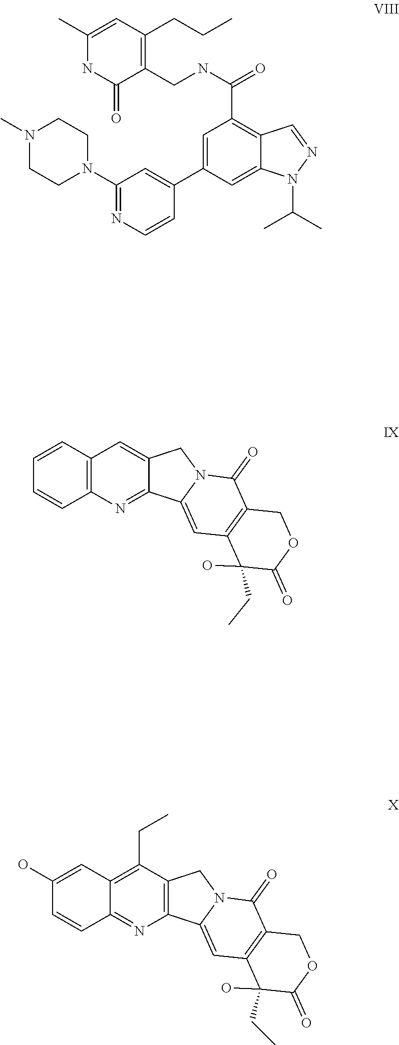 [SMILES: CCCc1cc(C)[nH]c(=O)c1CNC(=O)c1cc(-c2ccnc(N3CCN(C)CC3)c2)cc2c1cnn2C(C)C ; CC[C@@]1(O)C(=O)OCc2c1cc1n(c2=O)Cc2cc3ccccc3nc2-1 ; CCc1c2c(nc3ccc(O)cc13)-c1cc3c(c(=O)n1C2)COC(=O)[C@]3(O)CC]

6. The FUBP1 inhibitor for the use of any one of claims 1 to 3, wherein said inhibitor is a nucleic acid molecule of 12 to 60 nucleotides in length comprising or consisting of a contiguous nucleotide sequence of 12 to 30 nucleotides in length which is at least 95% complementary to a mammalian FUBP1 target nucleic acid and capable of reducing FUBP1 mRNA.

7. The nucleic acid molecule for the use of claim 6, wherein the nucleic acid molecule is selected from
a. a single stranded antisense oligonucleotide;
b. a siRNA molecule; or
c. a shRNA molecule.

8. The nucleic acid molecule for the use of claim 6 or 7, wherein the mammalian FUBP1 target nucleic acid is selected from SEQ ID NO: 1 to 8

9. The nucleic acid molecule for the use of any one of claims 6 to 8, wherein the contiguous nucleotide sequence is at least 98% complementarity to the target nucleic acid of SEQ ID NO: 1 and SEQ ID NO: 5.

10. The FUBP1 inhibitor for the use of any one of claims 1 to 9, wherein the cccDNA in an HBV infected cell is reduced by at least 60% when compared to a control.

11. The nucleic acid molecule for the use of any one of claims 6 to 9, wherein the FUBP1 mRNA is reduced by at least 60% when compared to a control.

12. A nucleic acid molecule of 12 to 60 nucleotides in length which comprises or consists of a contiguous nucleotide sequence of 12 to 30 nucleotides in length wherein the contiguous nucleotide sequence is at least 95% complementarity to a mammalian FUBP1 target nucleic acid.

13. The nucleic acid molecule of claim 12, wherein the nucleic acid molecule is a siRNA or a single stranded antisense oligonucleotide.

14. The nucleic acid molecule of claim 12 or 13, wherein the nucleic acid molecule comprises one or more 2′ sugar modified nucleosides and one or more phosphorthioate linkages.

15. The nucleic acid molecule of any one of claims 12 to 14, wherein contiguous nucleotide sequence is complementary to a target sequence selected from the group consisting of position 14200-14218, 14413-14431, 14966-14984 and 30344-30362 on SEQ ID NO: 1.

16. A conjugate compound comprising the nucleic acid molecule of any one of claims 12 to 15 and at least one conjugate moiety covalently attached to an oligonucleotide component of the nucleic acid molecule.

17. The conjugate compound of claim 16, wherein the conjugate moiety is selected from one of the trivalent GalNAc moieties in FIG. 1.

18. The conjugate compound of claim 16 or 17 comprising a physiologically labile linker composed of 2 to 5 linked nucleosides comprising at least two consecutive phosphodiester linkages, wherein the physiologically labile linker covalently bound at the 5′ or 3′ terminal of the oligonucleotide component.

19. A pharmaceutical composition comprising one or more nucleic acid molecule(s) of any one of claims 12 to 15, or one or more of the conjugate compound(s) of any one of claims 16 to 18, or acceptable salts thereof and a pharmaceutically acceptable diluent, carrier, salt and/or adjuvant.

20. An in vivo or in vitro method for modulating FUBP1 expression in a target cell which is expressing FUBP1, said method comprising administering the nucleic acid molecule of any one of claims 12 to 15, or the conjugate compound of any one of claims 16 to 18 or the pharmaceutical composition of claim 19 in an effective amount to said cell.

21. The nucleic acid molecule of any one of claims 12 to 15, or the conjugate compound of any one of claims 16 to 18 or the pharmaceutical composition of claim 19 for use as a medicament.
